(19) United States
(12) Patent Application Publication
(10) Pub. No.: US 2005/0042735 A1
(43) Pub. Date: Feb. 24, 2005

(54) Metabolic engineering for enhanced production of chitin and chitosan in microorganisms

(76) Inventors: Ming-De Deng, Manitowoc, WI (US); Thomas W. McMullin, Manitowoc, WI (US); Alan D. Grund, Manitowoc, WI (US)

Correspondence Address: SHERIDAN ROSS PC, 1560 BROADWAY, SUITE 1200, DENVER, CO, 80202, US

(21) Appl. No.: 10/823,397

(22) Filed: Apr. 12, 2004

Related U.S. Application Data
(60) Provisional application No. 60/462,087, filed on Apr. 11, 2003.

(51) Int. Cl. C12P 19/28; C12N 1/18; C12P 19/04

(52) U.S. Cl. 435/85; 435/254.2

(57) ABSTRACT

Disclosed is a fermentation method for the production of commercially useful amounts of chitin and/or chitosan by a biosynthetic process. Also disclosed are genetically modified microorganisms useful in such a method and a microbial biomass containing chitin and/or chitosan produced by such a method.

### CROSS REFERENCE TO RELATED APPLICATIONS

This application claims the benefit of priority under 35 U.S.C. 119(e) from U.S. Provisional Application Ser. No. 60/462,087, filed Apr. 11, 2003. The entire disclosure of U.S. Provisional Application Ser. No. 60/462,087 is incorporated herein by reference.

### FIELD OF INVENTION

The present invention relates to methods for increasing the levels of chitin and chitosan in microorganisms by metabolic engineering. The present invention also relates to genetically-modified strains of microorganisms useful for production of chitin and chitosan.

### BACKGROUND OF THE INVENTION

Chitin is a linear co-homopolymer of β-(1-4)-linked N-acetylglucosamine and glucosamine, the most abundant natural polymer next to cellulose. It is a constituent of the exoskeleton of arthropods such as shrimp and crabs. The cell wall of yeast and filamentous fungi also contain chitin and its more deacetylated form, chitosan. Natural chitin contains deacetylated amino sugar residues (glucosamine), but the degree of deacetylation is about 15% or lower. Degree of deacetylation in chitosan is normally much greater, generally over 70%. The primary commercial value of chitin is as the raw material for the preparation of glucosamine and chitosan. Unlike chitin, chitosan is water-soluble and has numerous nutritional, medical, pharmaceutical and industrial applications.

Currently, chitin is mainly extracted from raw materials like shrimp and crab shells. Chitosan is commercially-produced from chitin by hydrolysis with strong alkali at high temperature for long periods of time (Knorr, 1991). Commercial processes are arguably limited by raw material supply. It is also difficult to provide uniform and high quality product when starting with shellfish materials. For example, the degree of N-deacetylation varies according to the source of raw material and to the specific production process. Degree of N-deacetylation, molecular weight, viscosity and purity are key factors determining the suitability of chitosan products for different product applications.

Numerous publications report use of fungal biomass for production and recovery of chitosan. Methods of chitosan production from microbial biomass, such as filamentous fungi, were disclosed in U.S. Pat. No. 4,806,474 (Hershberger, 1985) and PCT application WO 01/68714 (Fan et al., 2000) and other publications (Rane and Hoover, 1993; Synowiecki and Al-Khateeb, 1997; Pochanavanich and Suntomsuk. 2002). However, these processes yield relatively expensive chitosan as compared to product extracted from shellfish. The yield of extracted chitosan is limited by the chitin and chitosan contents in the biomass. Favorably, the microbial biomass used in chitosan production is usually a by-product of fermentation processes for the production of other primary products and is, therefore, a nominal zero-cost starting material. This is offset by the low yield of chitosan resulting from extracting biomass with low chitin content. The ability of manufacturers to meet commercial growth of the chitosan market by increasing production of chitosan from waste-product biomass will be limited by the maximum production levels of the primary product. Increasing production capacity solely for the purpose of producing by-product, or for modifying the process to support a higher content of chitin in the biomass (likely at the expense of the primary process) is generally uneconomical.

Enzymes and their corresponding genes involved in the metabolic pathways leading to chitin and chitosan formation have been characterized (for reviews see Farkas, 1989 and Ronceo, 2002). Glutamine-fructose-6-P amidotransferase (also called glucosamine synthetase), encoded by the genes named GFA1 in eukaryotic organisms and glmS in bacteria, catalyzes the synthesis of glucosamine-6-P from fructose-6-P, the first step committed to chitin and chitosan synthesis. Bulik et al. (2003) and Lagorce et al. (2002) reported that this step is the target of regulation in chitin synthesis in the yeast, Saccharomyces cerevisiae. However, the authors focused only on transcription control of GFA1 gene expression and the enzyme activity. Overexpression of Gfalp enzyme led to a three-fold increase in chitin content in yeast. Despite the increase, the chitin content was too low, 2 to 3% of the dry cell weight, for economic commercial production of chitin and chitosan.

As compared to yeast, there are only a few reports in the literature about chitin and chitosan synthesis pathway in filamentous fungi. Borgia and Dodge (1992) characterized mutants of Aspergillus nidulans that were deficient in cell wall chitin and glucan. Muller et al. (2002) studied morphology changes in Aspergillus oryzae by gene disruption to alter chitin and chitosan synthesis. Maw et al. (2002) screened for clones of Gongronella butleri with high chitosan content following random mutagenesis. The authors reported a correlation of high chitosan content and higher chitin deacetylase activity. However, metabolic engineering of filamentous fungi with the goal of increasing chitin and chitosan levels has not been described. Moreover, any impact of GFA1 overexpression on the chitosan level in fungi has not been determined. In general, the Gfa1p from filamentous fungi has not been well studied and no fungal GFA1 genes have been fully cloned and characterized. A partial GFA1 sequence was identified in a cDNA clone from Aspergillus nidulans by sequence homology (GenBank Acc # CK447041).

It is desirable, therefore, to develop new strains of microorganisms that produce chitin and/or chitosan at substantially higher levels. Dedicated fermentation processes to make biomass with high concentrations of chitin will also make it possible to develop low cost fermentation processes for the production of high quality chitosan.

### SUMMARY OF THE INVENTION

A novel metabolic engineering approach is disclosed in the present invention for maximizing chitin and chitosan production in microorganisms. The metabolic engineering approach disclosed in the present invention can also be integrated into microbial strains used in existing fermentation processes in order to produce chitin and chitosan at higher levels in biomass by-products.

One embodiment of the present invention relates to a method to produce chitin or chitosan by a fermentation process. The method includes the steps of: (a) culturing in a fermentation medium a microorganism which comprises at least one genetic modification that affects the production of chitin and/or chitosan by the microorganism; and (b) collecting a product produced from the step of culturing which is selected from the group consisting of chitin and chitonase. The genetic modification can be selected from: (i) a genetic modification that results in an increase in the activity of glutamine-fructose-6-phosphate amidotransferase; (ii) a genetic modification that results in an increase in the activity of glucosamine-6-P acetyltransferase; (iii) a genetic modification that results in an increase in the activity of chitin synthase; (iv) a genetic modification that results in an increase in the activity of chitin deacetylase; (v) a genetic modification that results in a decrease in the activity of N-acetylglucosamine-6-P deacetylase; (vi) a genetic modification that results in a decrease in the activity of glucosamine-6-P deaminase; (vii) a genetic modification that results in a decrease in the activity of chitinase; and (viii) a genetic modification that results in a decrease in the activity of chitosanase.

In one aspect, the glutamine-fructose-6-P amidotransferase is resistant to inhibition by UDP-N-acetylglucosamine. In another aspect, the glutamine-fructose-6-P amidotransferase is resistant to inhibition by glucosamine-6-phosphate. In yet another aspect, the glutamine-fructose-6-P amidotransferase is resistant to inhibition by glutamate.

In one aspect, the microorganism has a genetic modification that increases the activity of glutamine-fructose-6-phosphate amidotransferase, and the genetic modification comprises transforming the microorganism with a recombinant nucleic acid molecule encoding the glutamine-fructose-6-phosphate amidotransferase, or with a biologically active homologue thereof. The recombinant nucleic acid molecule can, for example, comprise the coding region of yeast GFA1 or the coding region of bacterial GlmS. In one aspect, the glutamine-fructose-6-phosphate amidotransferase is resistant to inhibition by UDP-N-acetylglucosamine. In another aspect, the glutamine-fructose-6-phosphate amidotransferase is resistant to inhibition by glucosamine-6-phosphate. In one aspect, glutamine-fructose-6-phosphate amidotransferase is resistant to inhibition by glutamate.

In any of the above-described aspects of the method of the invention, the microorganism can further include a genetic modification that results in an increase in the activity of glucosamine-6-P acetyltransferase. In one aspect, the microorganism can include a genetic modification that results in an increase in the activity of chitin synthase. In another aspect, the microorganism can include a genetic modification that results in an increase in the activity of chitin deacetylase. In yet another aspect, the microorganism can include a genetic modification that results in an increase in the activity of chitin synthase and a genetic modification that results in an increase in the activity of chitin deacetylase.

In another aspect, the microorganism can include a genetic modification that results in a decrease in the activity of glucosamine-6-P dearminase. In yet another aspect, the microorganism can include a genetic modification that results in a decrease in the activity of N-acetylglucosamine-6-P deacetylase. In one aspect, the microorganism comprises a genetic modification that results in a decrease in the activity of N-acetylglucosamine-6-P deacetylase and a genetic modification that results in a decrease in the activity of glucosamine-6-P deaminase.

In another aspect, the microorganism comprises a genetic modification that results in a decrease in the activity of chitinase. In one aspect, the microorganism comprises a genetic modification that results in a decrease in the activity of chitosanase. In yet another aspect, the microorganism comprises a genetic modification that results in a decrease in the activity of chitinase and a genetic modification that results in a decrease in the activity of chitosanase.

In any of the above-described aspects of the method of the invention, the microorgansim can include, but is not limited to, any fungus. For example, the microorganism can be a yeast including, but not limited to, a yeast of the genus Saccharomyces or Schizosaccharomyces. In another aspect, the microorganism can be a filamentous fungus, including, but not limited to, a fungus of the genus Aspergillus, Absidia or Rhizopus. In one aspect, the microorganism is selected from: S. cerevisiae, A. niger, and A. nidulans.

Preferably, the genetic modifications increase the content of chitin or chitosan in the cell wall of the microorganism as compared to the wild-type microorganism by at least about 50%, and more preferably at least 100%, and more preferably at least about 2 fold, and more preferably at least about 5 fold, and even more preferably at least about 10 fold.

In one aspect of the method of the invention, the step of collecting comprises treatment of microorganism cells with a hot alkaline solution, collection and washing of the remaining solids containing chitin or chitosan, resuspension of the washed solids in an acidic solution to solubilize the chitin or chitosan, and precipitation of the chitin or chitosan.

Another embodiment of the present invention relates to a microbial biomass comprising chitin and/or chitosan and produced by the method of the present invention as described above.

Yet another embodiment of the present invention relates to a genetically modified microorganism as described above or in particular, comprising at least two genetic modifications selected from: (a) a genetic modification that results in an increase in the activity of glutamine-fructose-6-phosphate amidotransferase; (b) a genetic modification that results in an increase in the activity of glucosamine-6-P acetyltransferase; (c) a genetic modification that results in an increase in the activity of chitin synthase; (d) a genetic modification that results in an increase in the activity of chitin deacetylase; (e) a genetic modification that results in a decrease in the activity of N-acetylglucosamine-6-P deacetylase; (f) a genetic modification that results in a decrease in the activity of glucosamine-6-P deaminase; (g) a genetic modification that results in a decrease in the activity of chitinase; and (h) a genetic modification that results in a decrease in the activity of chitosanase.

### BRIEF DESCRIPTION OF THE FIGURES OF THE INVENTION

FIG. 1 is an illustration of the pathway for chitin and chitosan biosynthesis in S. cerevisiae.

### DETAILED DESCRIPTION OF THE INVENTION

The present invention relates to a biosynthetic method for the production of chitin and chitosan. Such a method includes fermentation of a genetically modified microorganism to produce chitin and chitosan and processes to recover the chitin and chitosan products. The present invention also relates to genetically-modified microorganisms, including, but not limited to, yeast and other fungi (e.g., S. cerevisiae and A. niger), that are useful for producing chitin and chitosan.

As used herein, the term chitin and poly-N-acetylglucosamine can be used interchangeably. Chitosan, deacetylated chitin, poly-glucosamine, co-polymer of N-acetylglucosamine and glucosamine are used interchangeably.

As will be discussed in detail below, even though many of the pathways and genes involved in the metabolic pathways for chitin and chitosan production in microorganisms have been elucidated, until the present invention, it was not known which of the many possible genetic modifications would be necessary to generate a microorganism that can produce commercially significant amounts of chitin and chitosan. Moreover, the combinations of novel genetic modifications for the production of chitin and chitosan as described herein had not previously been appreciated.

The novel methods of the present invention for production of chitin and chitosan by fermentation are inexpensive and can produce chitin and chitosan at higher yield and higher quality than methods currently used in the art. The methods of the present invention can be used in a process to economically produce chitin and chitosan as principal products. In addition, the method of the present invention can be incorporated into other fermentation processes, producing chitin and chitosan as biomass by-products at high yield.

Chitin and chitosan are synthesized in yeast and fungi. The cell wall of the yeast S. cerevisiae is mainly composed of two classes of macromolecules: mannoprotein and β-glucans. Chitin is also a vital constituent of the yeast cell wall, but it accounts for only about 1 to 3% of the cell wall dry weight. Cell wall accounts for 20 to 30% of the cell dry mass, and chitin content in yeast biomass is usually 0.2 to 1%. Chitosan is only found in the ascospore cell wall of yeast spores. However, cell wall damage caused by mutations affecting biosynthesis of β-1,3-glucans, mannans or cell component assembly resulted in significant increases in chitin content, reaching up to 2 to 3% of cell dry weight.

In contrast to yeast, chitin and chitosan constitute a much larger fraction of the cell wall in many filamentous fungi. Some fungi classified as Zygomycetes contain chitosan as a major fraction of the cell wall and septa. For example, chitosan accounted for as much as 23% and 27% of the biomass of A. niger IM13 and Absidia coerulea, respectively (McGahren et al., 1984; Velichkov and Georgiev, 1991). Chitosan from Rhizopus oryzae TISTR3189 and A. niger TISTR3245 biomass have a relatively high degree of N-deacetylation, 88% and 90%, respectively (Pochanavanich and Suntornsuk, 2002).

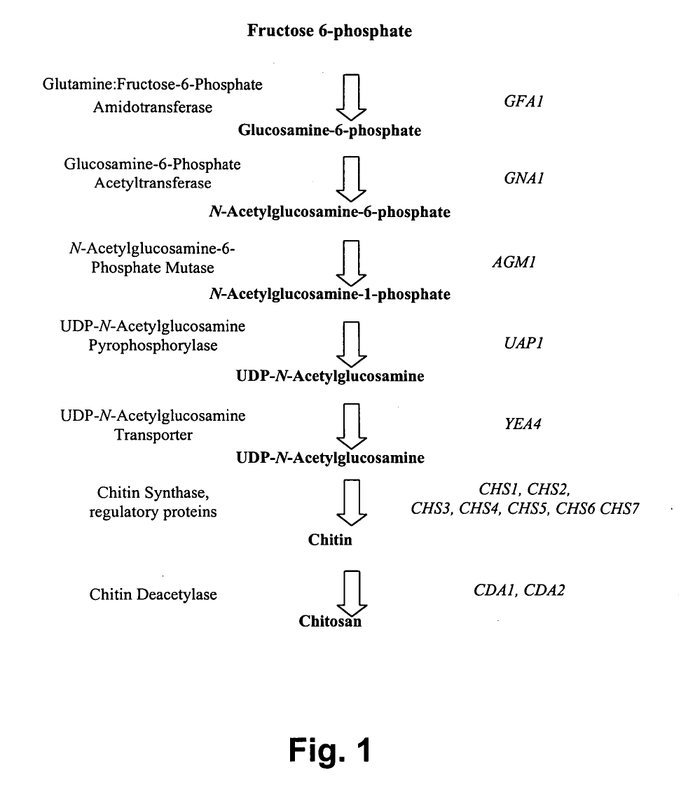

Genes and their products involved in the metabolic pathways leading to chitin and chitosan formation have been characterized in some microorganisms (Farkas, 1989; Ronceo, 2002). Chitin synthesis in the yeast cell wall has been investigated most extensively. FIG. 1 illustrates the general metabolic pathway for chitin and chitosan synthesis. The nomenclature of different enzymes and their alternative names can be found at the enzyme site of the ExPASy Molecular Biology Servers of the Swiss Institute of Bioinformatics. The nucleotide sequences of the identified or cloned genes and the amino acid sequences of their expression products are available in the databases at the National Center for Biotechnology Information and or in the ExPASy database.

The first dedicated step in chitin and chitosan pathway is the isomerization and amination of fructose-6-P to form glucosamine-6-P, a reaction catalyzed by glutamine-fructose-6-P amidotransferase (Gfalp; EC 2.6.1.16) encoded by GFA1. Glucosamine-6-P is N-acetylated to form N-acetylglucosamine-6-P by glucosamine-6-P acetyltransferase (EC 2.3.1.4) encoded by GNA1. Phospho-N-acetylglucosamine mutase (EC 5.4.2.3), encoded by AGM1 converts N-acetylglucosamine-6-P to N-acetylglucosamine-1-P, which is further converted to UDP-N-acetylglucosamine by UDP-N-acetylglucosaminepyrophosphorylase (EC 2.7.7.23), encoded by UAP1. The enzyme chitin synthase (EC 2.4.1.16) catalyzes polymerization of N-acetylglucosaminyl units by using UDP-N-acetylglucosamine as substrate. The reaction takes place on the plasma membrane. The enzyme utilizes UDP-N-acetylglucosamine present in the cytoplasm. The elongated polymer chains are extruded through the plasmalemma to the cell exterior. An UDP-N-acetylglucosamine transporter encoded by YEA4 is localized in the endoplasmic reticulum and it appears to be important to chitin synthesis. Chitin deacetylases (EC 3.5.1.41), coded by CDA1 and CDA2, convert nascent chitin to chitosan by hydrolyzing the acetyl group from amino sugar units; the enzyme is inactive with preformed chitin as substrate. This suggests that chitin synthase and chitin deacetylase operate consecutively for chitosan synthesis in filamentous fungi.

The number of chitin synthase isoenzymes varies from one copy in the yeast, S. pombe, to seven copies in some filamentous fungi, such as, A. fumigatus. The yeast, S. cerevisiae, contains three chitin synthases: Chs1p encoded by CHS1, Chs2p encoded by CHSII and Chs3p encoded by CHSIII. These synthases differ with respect to the peptide sequences, temporal and spatial expression patterns, and enzyme characteristics such as optimum pH, metal specificity, and susceptibility to inhibitors. Chs3p is responsible for the synthesis of 90 to 95% of the cellular chitin in yeast. Its optimal activity requires the involvement of four other regulatory proteins, encoded by CHS4 to CHS7, in its translocation and localization. Yeast strains defective in any of these genes have drastically reduced chitin synthesis.

Regulation of chitin and chitosan synthesis has not been well characterized prior to the present invention. Overexpression of individual CHS genes, or their combinations, is reported to have very marginal impact on chitin synthesis in yeast. However, cell wall damage caused by mutations affecting biosynthesis of β-1,3-glucans, mannans or cell component assembly resulted in a significant increase in chitin content in S. cerevisiae, reaching up to 2 to 3% of cell dry weight. Activation of chitin synthesis in cell-wall mutants is correlated with a 1.3 to 3.5-fold increase in the expression level of most of the chitin synthesis pathway genes (Lagorce et al., 2002; Bulik et al., 2003).

Four lines of evidence indicate that chitin synthesis is regulated by the availability of the precursor, UDP-N-acetylglucosamine. First, it was reported that the treatment of haploid yeast cells with mating pheromones caused a transcription activation of GFA1 and a proportional rise in chitin content (Schekman and Brawley, 1979). Cell-wall mutants had significantly elevated GFA1 expression, Gfalp activity and elevated chitin content, although the mechanisms of the changes are not known. Second, overexpressing the GFA1 gene by using a plasmid construct led to a three-fold increase in chitin level (Lagorce et al., 2002). Third, in a gfal mutant with 30% Gfalp activity, the increase in chitin content caused by the cell wall mutation was proportionally reduced. Fourth, growing cells in media supplemented with glucosamine resulted in an increase of three to four-fold in chitin levels in a dose-related fashion. Cell wall mutants grown with glucosamine in the medium showed a further increase in chitin content. Interestingly, the level of UDP-N-acetylglucosamine in the wild-type cells, grown in the presence of glucosamine, was increased by over 8-fold (Bulik et al., 2003).

It has yet to be demonstrated whether chitosan synthesis in filamentous fungi is also subject to regulation by precursors such as UDP-N-acetylglucosamine. Any impact of GFA1 overexpression on chitosan level in fungi has not been determined prior to the present invention. The Gfalp from filamentous fungi has not been well characterized and no fungal GFA1 genes have been fully cloned and characterized. However, sequence homology suggests that a cDNA clone from Aspergillus nidulans (GenBank Acc # CK447041) contained a partial GFA1 sequence.

Synthesis of glucosamine-6-P from fructose-6-P is the first step committed to chitin and chitosan synthesis. It has been concluded that this step is the major target of regulation in chitin synthesis. Overexpression of Gfalp enzyme has been shown to lead to a three-fold increase in chitin content in yeast, although this level of expression is too low for economic commercial production of chitin and chitosan. The control of chitosan synthesis in fungi appears to be more complex than in yeast. As discussed above, the number of isoenzymes for chitin synthase, another important enzyme involved in chitin and chitosan synthesis, varies from one copy in Schizosaccharomyces pombe and three copies in S. cerevisiae (yeast), to seven copies in some filamentous fungi such as Aspergillus fumigatus. Importantly, in addition to transcription control, the enzyme glutamine-fructose amidotransferase is strongly regulated allosterically (White 1968; Badet et al., 1988; McKnight et al., 1992; Broschat et al., 2002). The enzyme is strongly inhibited by glucosamine-6-P, N-acetyglucosamine-6-P and glutamate. Moreover, the eukaryotic enzyme, Gfalp, is inhibited by UDP-N-acetylglucosamine. As disclosed in U.S. Pat. No. 6,372,457, incorporated herein by reference in its entirety, feedback inhibition restricts glucosamine synthesis in E. coli. Overexpression of enzymes resistant to product inhibition led to dramatically increased levels of glucosamine production.

It is proposed herein that feedback inhibition plays an important role in controlling chitin and chitosan synthesis in yeast and fungi. Disclosed in the present invention are methods to relieve the feedback inhibition to increase chitin and chitosan production in microorganisms, principally by overexpressing Gfalp or GlmS enzymes (first step) resistant to feedback inhibition. Also disclosed in the present invention are methods to overcome other bottlenecks in the chitin and chitosan synthesis pathway in microorganisms once the limits on the first step are removed.

Glucosamine and N-acetylglucosamine are precursors for chitin and chitosan synthesis in different organisms. U.S. Pat. No. 6,372,457 and PCT Publication No. WO 2004/003175 A2, each of which is incorporated by reference in its entirety, disclose processes and materials for production of glucosamine and N-acetylglucosamine by microbial metabolic engineering and fermentation. However, these references do not disclose or suggest how metabolic engineering can be used to produce chitin and chitosan in microorganisms. Disclosed in the present application is an improvement to the glucosamine/N-acetylglucosamine metabolic engineering platform to enable the production of chitin and chitosan in microorganisms. It is the objective of the present invention to demonstrate significantly enhanced chitin and chitosan synthesis in filamentous fungi by metabolic engineering and fermentation.

Accordingly, one embodiment of the present invention relates to a method to produce chitin or chitosan by a fermentation process. The method includes the steps of: (a) culturing in a fermentation medium a genetically modified (genetically engineered) microorganism that has been engineered to have increased production of chitin or chitosan; and (b) collecting a product produced from the step of culturing which is selected from the group consisting of chitin and chitonase. The genetically modified microorganism comprises one or more genetic modifications selected from: (i) a genetic modification that results in an increase in the activity of glutamine-fructose-6-phosphate amidotransferase or a biologically active homologue thereof; (ii) a genetic modification that results in an increase in the activity of glucosamine-6-P acetyltransferase or a biologically active homologue thereof; (iii) a genetic modification that results in an increase in the activity of chitin synthase or a biologically active homologue thereof; (iv) a genetic modification that results in an increase in the activity of chitin deacetylase or a biologically active homologue thereof; (v) a genetic modification that results in a decrease in the activity of N-acetylglucosamine-6-P deacetylase; (vi) a genetic modification that results in a decrease in the activity of glucosamine-6-P deaminase; (vii) a genetic modification that results in a decrease in the activity of chitinase; and (viii) a genetic modification that results in a decrease in the activity of chitosanase. In one embodiment, the glutamine-fructose-6-P amidotransferase or homologue thereof is resistant to inhibition by UDP-N-acetylglucosamine. In another embodiment, the glutamine-fructose-6-P amidotransferase or homologue thereof is resistant to inhibition by glucosamine-6-phosphate. In yet another embodiment, the glutamine-fructose-6-P amidotransferase or homologue thereof is resistant to inhibition by glutamate. In one embodiment, the genetically modified microorganism comprises a genetic modification that results in an increase in the activity of chitin synthase and a genetic modification that results in an increase in the activity of chitin deacetylase. In another embodiment, the genetically modified microorganism comprises a genetic modification that results in a decrease in the activity of N-acetylglucosamine-6-P deacetylase and a genetic modification that results in a decrease in the activity of glucosamine-6-P deaminase. In yet another embodiment, the genetically modified microorganism comprises a genetic modification that results in a decrease in the activity of chitinase and a genetic modification that results in a decrease in the activity of chitosanase. Any one modification and preferably, any combination of two or more modifications described above is encompassed by the present invention.

Also included in the present invention is any microbial biomass comprising chitin and/or chitosan and produced by the method of the invention, as well as isolated genetically modified microorganisms useful in the method of the invention.

It is known in the art that the enzymes having the same biological activity may have different names depending on from what organism the enzyme is derived. The following is a general listing and discussion of alternate names for many of the enzymes referenced herein and specific names of genes encoding such enzymes from some organisms. The enzyme names can be used interchangeably, or as appropriate for a given sequence or organism, although the invention intends to encompass enzymes of a given function from any organism. Therefore, for example, while glucosamine-fructose-6-phosphate aminotransferase is the name typically used to refer to an enzyme in yeast and other fungi, general reference to “a glucosanine-fructose-6-phosphate aminotransferase” will be intended to refer to structural/functional homologues of the yeast enzyme from other types of microorganisms, plants and animals that are known in the art or to structural/functional homologues that are synthetically produced or produced by classical mutagenesis. For example, in bacteria, glucosamine-fructose-6-phosphate aminotransferase is commonly called glucosamine-6-phosphate synthase or glucosamine-6-phosphate synthetase. However, a general reference herein to glucosamine-fructose-6-phosphate aminotransferase without specifically identifying the source can include a bacterial glucosamine-6-phosphate synthase.

For example, the enzyme generally referred to herein as “glucosamine-6-phosphate synthase” catalyzes the formation of glucosamine-6-phosphate and glutamate from fructose-6-phosphate and glutamine. The enzyme is also known as glucosamine-fructose-6-phosphate aminotransferase (isomerizing); hexosephosphate aminotransferase; D-fructose-6-phosphate amidotransferase; glucosamine-6-phosphate isomerase (glutamine-forming); L-glutamine-fructose-6-phosphate amidotransferase; and GlcN6P synthase. The glucosamine-6-phosphate synthase from E. coli and other bacteria is generally referred to as GlmS. The glucosamine-6-phosphate synthase from yeast and other sources is generally referred to as GFA1 or GFAT.

Glucosamine-fructose-6-phosphate aminotransferases from a variety of organisms are known in the art and are contemplated for use in the genetic engineering strategies of the present invention. For example, the glucosamine-fructose-6-phosphate aminotransferase (GFA1) from Saccharomyces cerevisiae is described herein, and which has an amino acid sequence represented herein by SEQ ID NO:29, encoded by a nucleic acid sequence represented herein by SEQ ID NO:28. The glucosamine-fructose-6-phosphate aminotransferase from Escherichia coli is also described herein, which in bacteria is called glucosamine-6-phosphate synthase. The glucosamine-6-phosphate synthase from E. coli has an amino acid sequence represented herein by SEQ ID NO:23, which is encoded by a nucleic acid sequence represented herein by SEQ ID NO:22. Also described herein is the glucosamine-6-phosphate synthase from Bacillus subtilis, which has an amino acid sequence represented herein by SEQ ID NO:27, encoded by a nucleic acid sequence represented herein by SEQ ID NO:26. Glucosamine-fructose-6-phosphate aminotransferases (GFA1) from other microorganisms are also known in the art, such as from Candida albicans, which has an amino acid sequence represented herein by SEQ ID NO:31, encoded by a nucleic acid sequence represented herein by SEQ ID NO:30. Also included in the invention are glucosamine-fructose-6-phosphate aminotransferases which have one or more genetic modifications that produce a result chosen from: increased enzymatic activity of glucosamine-fructose-6-phosphate aminotransferase; reduced inhibition of the glucosamine-fructose-6-phosphate aminotransferase by UDP-N-acetylglucosamine; reduced inhibition of the glucosamine-fructose-6-phosphate aminotransferase by glucosamine-6-phosphate; reduced inhibition of the glucosamine-fructose-6-phosphate aminotransferase by glutamate; and increased affinity of glucosamine-fructose-6-phosphate aminotransferase for its substrates. In general, according to the present invention, an increase or a decrease in a given characteristic of a mutant or modified enzyme is made with reference to the same characteristic of a wild-type (i.e., normal, not modified) enzyme from the same organism which is measured or established under the same or equivalent conditions (discussed in more detail below). resistant to inhibition.

The enzyme generally referred to herein as glucosamine-6-phosphate acetyltransferase, converts glucosamine-6-phosphate and acetyl-CoA to N-acetylglucosamine-6-phosphate, releasing CoA. The enzyme is also known as glucosamine-phosphate N-acetyltransferase, phosphoglucosamine transacetylase and phosphoglucosamine acetylase. The yeast enzyme is generally referred to as GNA1. Glucosamine-6-phosphate acetyltransferases from a variety of organisms are known in the art and are contemplated for use in the genetic engineering strategies of the present invention. For example, the glucosamine-6-phosphate acetyltransferase from Saccharomyces cerevisiae is described herein. The glucosamine-6-phosphate acetyltransferase from Saccharomyces cerevisiae has an amino acid sequence represented herein by SEQ ID NO:33, which is encoded by a nucleic acid sequence represented herein by SEQ ID NO:32. Also described herein is the glucosamine-6-phosphate acetyltransferase from Candida albicans, which has an amino acid sequence represented herein by SEQ ID NO:35, encoded by a nucleic acid sequence represented herein by SEQ ID NO:34. Also included in the invention are glucosamine-6-phosphate acetyltransferases that have a genetic modification that produces a result selected from: increased enzymatic activity of glucosamine-6-phosphate acetyltransferase; overexpression of glucosamine-6-phosphate acetyltransferase by the microorganism; reduced N-acetylglucosamine-6-phosphate product inhibition of the glucosamine-6-phosphate acetyltransferase; and increased affinity of glucosamine-6-phosphate acetyltransferase for glucosamine-6-phosphate.

The enzyme generally referred to herein as glucosamine-6-phosphate deaminase catalyzes a reversible reaction of glucosamine-6-phosphate and water to form fructose-6-phosphate and ammonium. The enzyme is also known as glucosamine-6-phosphate isomerase; GlcN6P deaminase; phosphoglucosaminisomerase; phosphoglucosamine isomerase; glucosamine phosphate deaminase; 2-amino-2-deoxy-D-glucose-6-phosphate ketol isomerase (deaminating). Glucosamine-6-phosphate deaminases from a variety of organisms are known in the art and are contemplated for use in the genetic engineering strategies of the present invention. In E. coli and other bacteria, the enzyme is generally known as NagB. The enzyme from yeast such as Candida albicans is known as NAG1. The C. albicans NAG1 amino acid sequence is deposited as database Accession No. AAF04334.1 and is represented herein by SEQ ID NO:42. The genomic DNA sequence is included in database Accession No. AF079804 and is represented herein by SEQ ID NO:41. Also included in the invention are glucosamine-6-phosphate deaminases that have a genetic modification that produces a result selected from: decreased enzymatic activity of glucosamine-6-phosphate deaminase, increased reverse reaction of glucosamine-6-phosphate deaminase to form increased (more) glucosamine-6-phosphate, reduced forward reaction of glucosamine-6-phosphate deaminase to form reduced (less) fiuctose-6-phosphate, increased affinity of glucosamine-6-phosphate deaminase for fructose-6-phosphate, and reduced affinity of glucosamine-6-phosphate deaminase for glucosamine-6-phosphate. In a preferred embodiment, the gene or nucleic acid molecule encoding glucosamine-6-phosphate deaminase is mutated, inactivated or deleted to decrease or abolish the activity of the deaminase.

The enzyme generally referred to herein as N-acetylglucosamine-6-phosphate deacetylase hydrolyzes N-acetylglucosamine-6-phosphate to glucosamine-6-phosphate and acetate. N-acetylglucosamine-6-phosphate deacetylases from a variety of organisms are known in the art and are contemplated for use in the genetic engineering strategies of the present invention. The enzyme is known in E. coli and other bacteria as NagA. In yeast the enzyme is known as DAC1. The Candida albicans DAC1 amino acid sequence is at database Accession No. AAF04335.1, represented herein by SEQ ID NO:44, and its nucleotide sequence is included in database Accession No. AF079804, the full complement of which is represented herein by SEQ ID NO:43. Also included in the invention are N-acetylglucosamine-6-phosphate deacetylases that have a genetic modification that produces a result selected from: decreased activity of glucosamine-6-phosphate deacetylase; increased reverse reaction of glucosamine-6-phosphate deacetylase to form increased N-acetyl glucosamine-6-phosphate; reduced forward reaction of glucosamine-6-phosphate deacetylase to form reduced glucosamine-6-phosphate; increased affinity of glucosamine-6-phosphate deacetylase for glucosamine-6-phosphate; and reduced affinity of glucosamine-6-phosphate deacetylase for N-acetyl glucosamine-6-phosphate. In a preferred embodiment, the gene or nucleic acid molecule encoding glucosamine-6-phosphate deacetylase is mutated, inactivated or deleted to decrease or abolish the activity of the deacetylase.

The enzyme generally referred to herein as chitin synthase catalyzes the polymerization of N-acetylglucosamine using UDP-N-acetylglucosamine as donor. Chitin synthase can also be referred to as chitin-UDP acetyl-glucosaminyl transferase. Chitin synthase from a variety of organisms are known in the art and are contemplated for use in the genetic engineering strategies of the present invention. Numerous forms of chitin synthase enzymes and their nucleotide sequences have been identified in many different organisms, especially yeast and fungi. The amino acid and nucleotide sequences can be found in the NCBI and ExPASy databases. These include, but are not limited to, Saccharomyces cerevisiae CHS1 (amino acid sequence at database Accession Nos. P08004 or AAA34491.1, represented herein by SEQ. ID NO:46; encoded by SEQ ID NO:45 which is the coding sequence of database Accession No. M14045), CHS2 (database Accession No. P14180), CHS3 (database Accession No. P29465), CHS4 (also known as SKT5, database Accession No. NP_{—}009492), CHS5 (database Accession No. NP_{—}013434), CHS6 (database Accession No. NP_{—}012436), and CHS7. (database Accession No. NP_{—}012011); Aspergillus niger CHS1-ASPNG (the amino acid sequence for which is found in database Accession No. P30581, represented herein by SEQ ID NO:47) and CHS2-ASPNG (database Accession No. P30582); A. fumigatus CHSC_ASPFU (amino acid sequence at database Accession No. Q92197, represented herein by SEQ ID NO:49, encoded by SEQ ID NO:48, which is found in database Accession No. X94245), CHSD_ASPFU (database Accession No. P78746), and CHSG_ASPFU (database Accession No. P54267); and Aspergillus orzae chitin synthase (amino acid sequence at database Accession No. AAK31732.1, represented herein by SEQ ID NO:51, encoded by SEQ ID NO:50, which is found in database Accession No. AY029261), chsZ (database Accession No. BBB88127.1), and chsY (database Accession No. BAB88128.1). Also included in the invention are chitin synthases that have a genetic modification that produces a result selected from: increased enzymatic activity of chitin synthase; overexpression of chitin synthase by the microorganism; reduced product inhibition of the chitin synthase; and increased affinity of chitin synthase for UDP-N-acetylglucosamine.

The enzyme generally referred to herein as chitin deacetylase hydrolyses the N-acetyl group from amino sugar units of the nascent chitin to form chitosan (EC. 3.5.1.41). Chitin deacetylases from a variety of organisms are known in the art and are contemplated for use in the genetic engineering strategies of the present invention. For example, two chitin deacetylases from Saccharomyces cerevisiae is described herein. The chitin deacetylase from S. cerevisiae known as CDA1 has an amino acid sequence represented herein by SEQ ID NO:37, which is encoded by a nucleic acid sequence represented herein by SEQ ID NO:36. The chitin deacetylase from S. cerevisiae known as CDA2 has an amino acid sequence represented herein by SEQ ID NO:39, which is encoded by a nucleic acid sequence represented herein by SEQ ID NO:38. The fungal chitin deacetylase amino acid and nucleotide sequence from Muccor rouxii are represented in by database Accession No. Z19109 (the nucleotide coding sequence is represented herein by SEQ ID NO:52, which encodes SEQ ID NO:53). The fungal chitin deacetylase amino acid and nucleotide sequences from Gongronella butleri are described in database Accession Nos. AAN65362 and AF411810; the nucleotide coding sequence is represented herein by SEQ ID. NO:54, which encodes SEQ ID NO:55. Also included in the invention are chitin deacetylases that have a genetic modification that produces a result selected from: increased enzymatic activity of chitin deacetylase; overexpression of chitin deacetylase by the microorganism; reduced product inhibition of the chitin deacetylase; and increased affinity of chitin deacetylase for chitin.

The enzyme generally referred to herein as chitinase depolymerizes chitin. Chitinase (EC 3.2.1.14) can also be referred to as chitodextrinase, 1,4-beta-poly-N-acetylglucosaminenidase, poly-beta-glucosamimidase. Chitinases from a variety of organisms are known in the art and are contemplated for use in the genetic engineering strategies of the present invention. A yeast (S. cerevisiae) chitinase amino acid and nucleotide sequences (database Accession No. M74070) are found in the sequence databases. The nucleotide sequence for the coding region of this chitinase is represented herein by SEQ ID NO:56, which encodes SEQ ID NO:57. Also included in the invention are chitinases that have a genetic modification that produces a result selected from: decreased activity of chitinase; and reduced affinity of chitinase for chitin. In a preferred embodiment, the gene or nucleic acid molecule encoding chitinase is mutated, inactivated or deleted to decrease or abolish the activity of the chitinase.

The enzyme generally referred to herein as chitosanase depolymerizes chitosan. Chitosanases (3.2.1.132) from a variety of organisms are known in the art and are contemplated for use in the genetic engineering strategies of the present invention. Examples are database Accession No. L40408 (Nocardioides sp.; nucleotide coding sequence represented herein by SEQ ID NO:58, encoding SEQ ID NO:59), database Accession No. D10624 (Bacillus circulans) and database. Accession No. L07779 (Streptomyces sp.). Also included in the invention are chitosanases that have a genetic modification that produces a result selected from: decreased activity of chitosanase; and reduced affinity of chitosanase for chitosan. In a preferred embodiment, the gene or nucleic acid molecule encoding chitosanase is mutated, inactivated or deleted to decrease or abolish the activity of the chitosanase.

To the extent that genes, other nucleic acid sequences, and amino acid sequences from a particular microorganism are discussed and/or exemplified below, it will be appreciated that other microorganisms have similar metabolic pathways, as well as genes and proteins having similar structure and function within such pathways. As such, the principles discussed below with regard to any particular microorganism, either as a source of genetic material or a host cell to be modified, are applicable to other microorganisms and are expressly encompassed by the present invention.

GFA1 and glmS genes encoding product-resistant forms of Gfalp and GlmS enzymes, respectively, can be created by mutagenesis and/or enzyme engineering. For example, product resistant variants of E. coli GlmS were previously successfully created by error-prone PCR and identified by plate assay for increased glucosamine production, as disclosed in U.S. Pat. No. 6,372,457, supra. GFA1 and glmS genes encoding product-resistant enzymes can also be isolated from native strains. The inventors have also previously demonstrated in PCT Publication No. WO 04/003175A2 that the glmS gene isolated from Bacillus subtilis strain. ATCC 23856 encodes an enzyme that is strongly resistant to glucosamine-6-P inhibition. When the Bacillus GlmS enzyme and the E. coli wild-type GlmS enzymes were overexpressed in E. coli, expression of the Bacillus enzyme resulted in a two-fold higher level of glucosamine production than the E. coli enzyme.

A downstream product, UDP-N-acetylglucosamine, inhibits the eukaryotic Gfalp enzyme (McKnight et al., 1992). Feedback inhibition could restrict the flow of pathway intermediates and could limit the level of chitin synthesis in vivo. As such, a mere overexpression of Gfalp enzyme should have only limited impact on chitin synthesis levels, due to feedback inhibition. Therefore, in one embodiment of the present invention, a genetically-modified microorganism for chitin and chitosan production includes a microorganism that has been transformed with a GFA1 nucleic acid molecule (e.g., a GFA1 gene or another Gfalp-encoding nucleic acid molecule), or nucleic acid molecule encoding a homologue of the GFA1 gene product (Gfalp), wherein the enzyme encoded thereby is less sensitive or more preferably, is not sensitive to inhibition by UDP-N-acetylglucosamine. Such a GFA1 nucleic acid molecule can be a native, resistant, eukaryotic GFA1 gene or nucleic acid molecule; a mutant or engineered eukaryotic GFA1 gene or nucleic acid molecule; a bacterial gene homologue of GFA1 (the bacterial form being referred to as glmS) or a nucleic acid molecule encoding GlmS; or a mutant and/or engineered bacterial glmS gene or nucleic acid molecule encoding a mutant and/or engineered GlmS, such as E. coli glmS*54 disclosed in U.S. Pat. No. 6,372,457, supra.

Gfa1p and GlmS enzymes are also inhibited by their product, glucosamine-6-P (White et al., 1968; Broschat et al., 2002). U.S. Pat. No. 6,372,457 disclosed that feedback inhibition is a critical factor limiting the synthesis of glucosamine. This patent described mutant E. coli glmS nucleic acid molecules and proteins that have drastically reduced sensitivity to product inhibition. Overexpression of the mutant enzymes, such as GlmS*54, resulted in much higher glucosamine production as compared to overexpression of the wild-type E. coli GlmS enzyme. Therefore, in another embodiment of the present invention, a genetically-modified microorganism for chitin and chitosan production includes a microorganism that is transformed with a GFA1 gene or nucleic acid molecule encoding a GFA1 gene product, or a nucleic acid molecule encoding a homologue of Gfa1p, wherein the enzyme encoded thereby is resistant to inhibition by glucosamine-6-P. Such a GFA1 nucleic acid molecule can be a native, resistant, eukaryotic GFA1 gene or other Gfalp-encoding nucleic acid molecule; a mutant or engineered eukaryotic GFA1 gene or nucleic acid molecule; a bacterial g/ms or nucleic acid molecule encoding GlmS; or a mutant and/or engineered bacterial glmS or nucleic acid molecule encoding a mutant and/or engineered bacterial GlmS.

The synthesis of glucosamine-6-P from fructose-6-P catalyzed by Gfa1p and GlmS requires glutamine as the amino donor, which is converted to glutamate. It was reported that the GlmS enzyme is subject to inhibition by glutamate (Badet et al., 1988). Accordingly, in another embodiment of the invention, a genetically-modified microorganism for chitin and chitosan production includes a microorganism that is transformed with a GFA1 gene or nucleic acid molecule encoding the GFA1 gene product, or a homologue thereof, whereby the encoded enzyme is resistant to inhibition by glutamate. Such a GFA1 gene or nucleic acid molecule can be a native, resistant, eukaryotic GFA1 gene or nucleic acid molecule encoding Gfa1p; a mutant or engineered eukaryotic GFA1 or nucleic acid molecule encoding Gfa1p; a bacterial glmS or nucleic acid molecule encoding GlmS, or a mutant and/or engineered bacterial glmS or nucleic acid molecule encoding a mutant and/or engineered GlmS.

The reaction catalyzed by Gfa1p appears to be the limiting step in chitin and chitosan synthesis in normal cells. However, once the restriction (bottleneck) of this step has been removed by an adequate overexpression of a GFA1p homologue that is resistant to inhibition by UDP-N-acetylglucosamine, glucosamine-6-P and/or glutamate, as described above, other steps in the pathway will likely become a bottleneck for further improvement in chitin and chitosan production. Therefore, another embodiment of the present invention includes approaches to overexpress other enzymes involved in the chitin and chitosan pathway. The target enzymes include, but are not limited to, glucosamine-6-P acetyltransferase, phospho-N-acetylglucosamine mutase, UDP-N-acetylglucosaminepyro-phosphorylase, UDP-N-acetylglucosamine transporter, chitin synthase, chitin synthase regulatory proteins and chitin deacetylase.

Chitin synthase catalyzes the polymerization of N-acetylglucosamine using UDP-N-acetylglucosamine as donor, while chitin deacetylase hydrolyses the N-acetyl group from amino sugar units of the nascent chitin to form chitosan. Chitin synthase and chitin deacetylase operate consecutively for chitosan synthesis in filamentous fingi. Coordination of chitin synthase action and chitin deacetylase appears to be a major factor determining the degree of deacetylation, which is an important characteristic of chitosan products. Maw et al. (2002) selected fungus Gongronella butleri strains producing chitosan at higher levels using UV mutagenesis. The authors reported that high chitosan was associated with high chitin deacetylase activity. The wild-type strain contained about 6% chitosan in the mycelia mass. CDA activity and chitosan level were doubled in some mutant clones. As another embodiment of the present invention, a genetically-modified microorganism for chitin and chitosan production includes a microorganism in which the expression of the chitin synthase and/or chitin deacetylase is increased or modulated for production of chitosan with a high degree of deacetylation at high yield.

In any of the embodiments described herein, a target enzyme can be provided in its native form (e.g., by overexpressing the native form of the enzyme) or in a mutant or engineered form for optimal performance (i.e. with increased affinity to the substrate, increased velocity, increased stability and other favorable characteristics). These embodiments are discussed in detail below.

Microorganisms have the capacity to use amino sugars, such as glucosamine and N-acetylglucosamine, as carbon sources. Amino sugar catabolism involves enzymes such as N-acetylglucosamine-6-P deacetylase and glucosamine-6-P deaminase. N-acetylglucosamine-6-P deacetylase hydrolyses N-acetylglucosamine-6-P to form glucosamine-6-P and acetate. The amino group is removed by glucosamine-6-P deaminase to form fructose-6-P and ammonium. In yet another embodiment of the present invention, a genetically-modified microorganism for chitin and chitosan production includes a microorganism in which the genes encoding for N-acetylglucosamine-6-P deacetylase and glucosamine-6-P deaminase are mutated, inactivated or deleted.

Yeast and other fungi have chitinase and chitosanase that depolymerize chitin and chitosan, respectively. Therefore, in another embodiment of the present invention, a genetically-modified microorganism for chitin and chitosan production includes a microorganism in which the expression and activity of chitinase and/or chitosanase are modulated for down-regulation, mutated, inactivated or deleted for production of chitin and chitosan at high yield and high quality.

In another embodiment of the present invention, a genetically-modified microorganism for chitin and chitosan production includes a microorganism that has the targeted genetic modifications (such as gene overexpression and gene deletion described above) and that is further improved by random mutagenesis.

In general, a microorganism having a genetically modified (also referred to as genetically engineered) metabolic pathway for the production of chitin and/or chitosan has at least one genetic modification, as discussed in detail below, which results in a change in one or more genes, enzymatic reactions, or pathways as described above as compared to a wild-type microorganism cultured under the same conditions. Such a modification a microorganism changes the ability of the microorganism to produce chitin and/or chitosan. As discussed in detail below, according to the present invention, a genetically modified microorganism preferably has an enhanced ability to produce chitin and/or chitosan as compared to a wild-type microorganism of the same species (and preferably the same strain), which is cultured under the same or equivalent conditions. Equivalent conditions are culture conditions which are similar, but not necessarily identical (e.g., some changes in medium composition, temperature, pH and other conditions can be tolerated), and which do not substantially change the effect on microbe growth or production of chitin or chitosan by the microbe.

Therefore, having generally described some of the preferred modifications according to the invention, in one embodiment, the microorganism comprises at least one genetic modification that increases the activity of glutamine-fructose-6-phosphate amidotransferase. Preferably, the genetic modification to increase the activity of the glutamine-fructose-6-phosphate amidotransferase produces a result selected from: increased enzymatic activity of glutamine-fructose-6-phosphate amidotransferase; overexpression of the glutamine-fructose-6-phosphate amidotransferase; reduced product inhibition of the glutamine-fructose-6-phosphate amidotransferase; and increased affinity of glutamine-fructose-6-phosphate amidotransferase for its substrates. In one aspect, the microorganism is transformed with at least one recombinant nucleic acid molecule comprising a nucleic acid sequence encoding the glutamine-fructose-6-phosphate amidotransferase (including a homologue of a naturally occurring glutamine-fructose-6-phosphate amidotransferase). Such a nucleic acid molecule can include a nucleic acid sequence encoding a glutamine-fructose-6-phosphate amidotransferase that has at least one genetic modification that increases the enzymatic activity of the glutamine-fructose-6-phosphate amidotransferase, that reduces the product inhibition of the glutamine-fructose-6-phosphate amidotransferase, or produces any of the above-described results. The function and representative sequences for glutamine-fructose-6-phosphate amidotransferases have been described above.

In another embodiment, the microorganism comprises at least one genetic modification that increases the activity of glucosamine-6-phosphate acetyltransferase in the microorganism. Preferably, the genetic modification to increase the activity of glucosamine-6-phosphate acetyltransferase provides a result selected from: increased enzymatic activity of glucosamine-6-phosphate acetyltransferase; overexpression of glucosamine-6-phosphate acetyltransferase by the microorganism; reduced N-acetyl glucosamine-6-phosphate product inhibition of the glucosamine-6-phosphate acetyltransferase; and/or increased affinity of glucosamine-6-phosphate acetyltransferase for glucosamine-6-phosphate. In one aspect, the microorganism is transformed with at least one recombinant nucleic acid molecule comprising a nucleic acid sequence encoding the glucosamine-6-phosphate acetyltransferase (including a homologue of a naturally occurring glucosamine-6-phosphate acetyltransferase). Such a nucleic acid molecule can include a nucleic acid sequence encoding a glucosamine-6-phosphate acetyltransferase that has at least one genetic modification that increases the enzymatic activity of the glucosamine-6-phosphate acetyltransferase, or produces any of the above-described results. The function and representative sequences for glucosamine-6-phosphate acetyltransferases have been described above.

In another embodiment, the microorganism comprises at least one genetic modification that increases the activity of chitin synthase in the microorganism. Preferably, the genetic modification to increase the activity of chitin synthase provides a result selected from: increased enzymatic activity of chitin synthase; overexpression of chitin synthase by the microorganism; reduced product inhibition of the chitin synthase; and/or increased affinity of chitin synthase for its substrate. In one aspect, the microorganism is transformed with at least one recombinant nucleic acid molecule comprising a nucleic acid sequence encoding the chitin synthase. Such a nucleic acid molecule can include a nucleic acid sequence encoding a chitin synthase (including a homologue of a naturally occurring chitin synthase) that has at least one genetic modification that increases the enzymatic activity of the chitin synthase, or produces any of the above-described results. The function and representative sequences for chitin synthases have been described above.

In another embodiment, the microorganism comprises at least one genetic modification that increases the activity of chitin deacetylase in the microorganism. Preferably, the genetic modification to increase the activity of chitin deacetylase provides a result selected from: increased enzymatic activity of chitin deacetylase; overexpression of chitin deacetylase by the microorganism; reduced product inhibition of the chitin deacetylase; and/or increased affinity of chitin deacetylase for its substrate. In one aspect, the microorganism is transformed with at least one recombinant nucleic acid molecule comprising a nucleic acid sequence encoding the chitin deacetylase (including a homologue of a naturally occurring chitin deacetylase). Such a nucleic acid molecule can include a nucleic acid sequence encoding a chitin deacetylase that has at least one genetic modification that increases the enzymatic activity of the chitin deacetylase, or produces any of the above-described results. The function and representative sequences for chitin deacetylases have been described above.

In another embodiment, the microorganism comprises at least one genetic modification that decreases the activity of glucosamine-6-phosphate deaminase. In one aspect, the genetic modification to decrease the activity of glucosamine-6-phosphate deaminase is a partial or complete deletion or inactivation of an endogenous gene encoding glucosamine-6-phosphate deaminase in the microorganism.

In another embodiment, the genetically modified microorganism comprises at least one genetic modification that decreases the activity of N-acetylglucosamine-6-phosphate deacetylase. In one aspect, the genetic modification to decrease the activity of N-acetylglucosamine-6-phosphate deacetylase is a partial or complete deletion or inactivation of an endogenous gene encoding N-acetylglucosamine-6-phosphate deacetylase in the microorganism.

In another embodiment, the microorganism comprises at least one genetic modification that decreases the activity of chitinase. In one aspect, the genetic modification to decrease the activity of chitinase is a partial or complete deletion or inactivation of an endogenous gene encoding chitinase in the microorganism.

In another embodiment, the microorganism comprises at least one genetic modification that decreases the activity of chitosanase. In one aspect, the genetic modification to decrease the activity of chitosanase is a partial or complete deletion or inactivation of an endogenous gene encoding chitosanase in the microorganism.

Various of the above-identified genetic modifications can be combined to produce microorganisms having more than one modification, as desired to enhance the production of chitin and/or chitosan by the microorganism.

Development of a microorganism with enhanced ability to produce chitin and/or chitosan by genetic modification can be accomplished using both classical strain development (see Examples) and/or molecular genetic techniques (see Examples). In general, the strategy for creating a microorganism with enhanced chitin and/or chitosan production is to (1) inactivate or delete at least one, and preferably more than one of the metabolic pathways in which production of chitin and/or chitosan is negatively affected (e.g., inhibited), and (2) amplify at least one, and preferably more than one of the metabolic pathways in which chitin and/or chitosan production is enhanced. Such modifications of pathways have been discussed in detail above.

As one embodiment of the present invention, to increase or change the biological activity of a particular enzyme, a mutagenized form of enzyme or an over-expressed enzyme can be produced and/or used. An enzyme with such improvements can be isolated from nature or produced by any suitable method of genetic modification or protein engineering. For example, computer-based protein engineering can be used for this purpose. See for example, Maulik et al., 1997, Molecular Biotechnology: Therapeutic Applications and Strategies, Wiley-Liss, Inc., which is incorporated herein by reference in its entirety. Amplification of the expression of the enzyme can be accomplished in the host microorganism, for example, by introduction of a recombinant nucleic acid molecule encoding the enzyme. Therefore, a gene encoding modified enzyme or other protein useful in the present invention can be a mutated (i.e., genetically modified) gene, for example, and can be produced by any suitable method of genetic modification. For example, a recombinant nucleic acid molecule encoding the enzyme can be modified by any method for inserting, deleting, and/or substituting nucleotides, such as by error-prone PCR. In this method, the gene is amplified under conditions that lead to a high frequency of misincorporation errors by the DNA polymerase used for the amplification. As a result, a high frequency of mutations is obtained in the PCR products. The resulting gene mutants can then be screened for by testing the mutant genes for the ability to confer increased chitin or chitosan production onto a test microorganism, as compared to a microorganism carrying the non-mutated recombinant nucleic acid molecule. The mutant variants of an enzyme could also be screened by the production of an intermediate of the chitin/chitosan pathway by using suitable detection methods. Therefore, it is an embodiment of the present invention to provide a microorganism which is transformed with a genetically modified recombinant nucleic acid molecule comprising a nucleic acid sequence encoding mutant, or homologue, enzymes as described herein. Homologues are described in detail below.

As described above, to produce significantly high yields of chitin and/or chitosan by the fermentation method of the present invention, a microorganism is genetically modified to enhance production of chitin and/or chitosan. As used herein, a genetically modified microorganism has a genome that is modified (i.e., mutated or changed) from its normal (i.e., wild-type or naturally occurring) form. In one aspect, such an organism can endogenously contain and express a gene encoding the protein of interest, and the genetic modification can be a genetic modification of the gene, whereby the modification has some effect (e.g., increase, decrease, delete) on the expression and/or activity of the gene. In another aspect, such an organism can endogenously contain and express a gene encoding the protein of interest, and the genetic modification can be an introduction of at least one exogenous nucleic acid sequence (e.g., a recombinant nucleic acid molecule), wherein the exogenous nucleic acid sequence encodes the protein of interest and/or a protein that affects the activity of the protein or gene encoding the protein. The exogenous nucleic acid molecule to be introduced into the microorganism can encode a wild-type protein or it can have one or more modifications that affect the expression and/or activity of the encoded protein as compared to the wild-type or normal protein. In yet another aspect, the organism does not necessarily endogenously (naturally) contain the gene encoding the protein of interest, but is genetically modified to introduce at least one recombinant nucleic acid molecule encoding a protein having the biological activity of the protein of interest. Again, the recombinant nucleic acid molecule can encode a wild-type protein or the recombinant nucleic acid sequence can be modified to affect the expression and/or activity of the encoded protein as compared to a wild-type protein. In other embodiments, various expression control sequences (e.g., promoters) can be introduced into the microorganism to effect the expression of an endogenous gene in the microorganism. Various embodiments associated with each of these aspects will be discussed in greater detail below.

As used herein, a genetically modified microorganism can include any genetically modified microorganism, including a bacterium, a protist, a microalgae, a fungus, or other microbe. Such a genetically modified microorganism has a genome which is modified (i.e., mutated or changed) from its normal (i.e., wild-type or naturally occurring) form and/or is modified to express extrachromosomal genetic material (e.g., a recombinant nucleic acid molecule), such that the desired result is achieved (e.g., increased, decreased, or otherwise modified enzyme expression and/or activity and/or modified production of chitin and/or chitosan as a result of the modification(s)). More particularly, the modification to the microorganism can be achieved by modification of the genome of the microorganism (e.g., endogenous genes) and/or by introducing genetic material (e.g., a recombinant nucleic acid molecule) into the microorganism, which can remain extrachromosomal or can be integrated into the host microbial genome. As such, the genetic modification can include the introduction or modification of regulatory sequences which regulate the expression of endogenous or recombinantly introduced nucleic acid sequences in the microorganism, the introduction of wild-type or modified recombinant nucleic acid molecules (e.g., encoding wild-type or modified proteins), the modification of endogenous genes in the microorganism, or any other modification which results in the microorganism having the specified characteristics with regard to enzyme expression and/or biological activity. Genetic modification of a microorganism can be accomplished using classical strain development and/or molecular genetic techniques. Such techniques known in the art and are generally disclosed for microorganisms, for example, in Sambrook et al., 1989, Molecular Cloning: A Laboratory Manual, Cold Spring Harbor. Labs Press. The reference Sambrook et al., ibid., is incorporated by reference herein in its entirety. A genetically modified microorganism can include a microorganism in which nucleic acid molecules have been inserted, deleted and/or modified (i.e., mutated; e.g., by insertion, deletion, substitution, and/or inversion of nucleotides), in such a manner that such modifications provide the desired effect within the microorganism. According to the present invention, a genetically modified microorganism includes a microorganism that has been modified using recombinant technology.

In one embodiment of the present invention, a genetic modification of a microorganism increases or decreases the activity of a protein involved in at least one metabolic pathway according to the present invention. Such a genetic modification includes any type of modification and specifically includes modifications made by recombinant technology and/or by classical mutagenesis. As used herein, genetic modifications which result in a decrease in gene expression, in the function of the gene, or in the function of the gene product (i.e., the protein encoded by the gene) can be referred to as inactivation (complete or partial), deletion, interruption, blockage, silencing or down-regulation of a gene. For example, a genetic modification in a gene which results in a decrease in the function of the protein encoded by such gene, can be the result of a complete deletion of the gene (i.e., the gene does not exist, and therefore the protein does not exist), a mutation in the gene which results in incomplete or no translation of the protein (e.g., the protein is not expressed), or a mutation in the gene which decreases or abolishes the natural function of the protein (e.g., a protein is expressed which has decreased or no enzymatic activity or action). More specifically, reference to decreasing the action or activity of enzymes discussed herein generally refers to any genetic modification in the microorganism in question which results in decreased expression and/or functionality (biological activity) of the enzymes and includes decreased activity of the enzymes (e.g., specific activity), increased inhibition or degradation of the enzymes as well as a reduction or elimination of expression of the enzymes. For example, the action or activity of an enzyme of the present invention can be decreased by blocking or reducing the production of the enzyme, reducing enzyme activity, or inhibiting the activity of the enzyme. Combinations of some of these modifications are also possible. Blocking or reducing the production of an enzyme can include placing the gene encoding the enzyme under the control of a promoter that requires the presence of an inducing compound in the growth medium. By establishing conditions such that the inducer becomes depleted from the medium, the expression of the gene encoding the enzyme (and therefore, of enzyme synthesis) could be turned off. Blocking or reducing the activity of an enzyme could also include using an excision technology approach similar to that described in U.S. Pat. No. 4,743,546, incorporated herein by reference. To use this approach, the gene encoding the enzyme of interest is cloned between specific genetic sequences that allow specific, controlled excision of the gene from the genome. Excision could be prompted by, for example, a shift in the cultivation temperature of the culture, as in U.S. Pat. No. 4,743,546, or by some other physical or nutritional signal.

Genetic modifications that result in an increase in gene expression or function can be referred to as amplification, overproduction, overexpression, activation, enhancement, addition, or up-regulation of a gene. More specifically, reference to increasing the action (or activity) of enzymes or other proteins discussed herein generally refers to any genetic modification in the microorganism in question which results in increased expression and/or functionality (biological activity) of the enzymes or proteins and includes higher activity of the enzymes (e.g., specific activity or in vivo enzymatic activity), reduced inhibition or degradation of the enzymes and overexpression of the enzymes. For example, gene copy number can be increased, expression levels can be increased by use of a promoter that gives higher levels of expression than that of the native promoter, or a gene can be altered by genetic engineering or classical mutagenesis to increase the biological activity of an enzyme. Combinations of some of these modifications are also possible.

In general, according to the present invention, an increase or a decrease in a given characteristic of a mutant or modified enzyme (e.g., enzyme activity) is made with reference to the same characteristic of a wild-type (i.e., normal, not modified) enzyme that is derived from the same organism (from the same source or parent sequence), which is measured or established under the same or equivalent conditions. Similarly, an increase or decrease in a characteristic of a genetically modified microorganism (e.g., expression and/or biological activity of a protein, or production of a product) is made with reference to the same characteristic of a wild-type microorganism of the same species, and preferably the same strain, under the same or equivalent conditions. Such conditions include the assay or culture conditions (e.g., medium components, temperature, pH, etc.) under which the activity of the protein (e.g., expression or biological activity) or other characteristic of the microorganism is measured, as well as the type of assay used, the host microorganism that is evaluated, etc. As discussed above, equivalent conditions are conditions (e.g., culture conditions) which are similar, but not necessarily identical (e.g., some conservative changes in conditions can be tolerated), and which do not substantially change the effect on microbe growth or enzyme expression or biological activity as compared to a comparison made under the same conditions.

Preferably, a genetically modified microorganism that has a genetic modification that increases or decreases the activity of a given protein (e.g., an enzyme) has an increase or decrease, respectively, in the activity (e.g., expression, production and/or biological activity) of the protein, as compared to the activity of the wild-type protein in a wild-type microorganism, of at least about 5%, and more preferably at least about 10%, and more preferably at least about 15%, and more preferably at least about 20%, and more preferably at least about 25%, and more preferably at least about 30%, and more preferably at least about 35%, and more preferably at least about 40%, and more preferably at least about 45%, and more preferably at least about 50%, and more preferably at least about 55%, and more preferably at least about 60%, and more preferably at least about 65%, and more preferably at least about 70%, and more preferably at least about 75%, and more preferably at least about 0.80%, and more preferably at least about 85%, and more preferably at least about 90%, and more preferably at least about 95%, or any percentage, in whole integers between 5% and 100% (e.g., 6%, 7%, 8%, etc.). The same differences are preferred when comparing an isolated modified nucleic acid molecule or protein directly to the isolated wild-type nucleic acid molecule or protein (e.g., if the comparison is done in vitro as compared to in vivo).

In another aspect of the invention, a genetically modified microorganism that has a genetic modification that increases or decreases the activity of a given protein (e.g., an enzyme) has an increase or decrease, respectively, in the activity (e.g., expression, production and/or biological activity) of the protein, as compared to the activity of the wild-type protein in a wild-type microorganism, of at least about 2-fold, and more preferably at least about 5-fold, and more preferably at least about 10-fold, and more preferably at least about 20-fold, and more preferably at least about 30-fold, and more preferably at least about 40-fold, and more preferably at least about 50-fold, and more preferably at least about 75-fold, and more preferably at least about 100-fold, and more preferably at least about 125-fold, and more preferably at least about 150-fold, or any whole integer increment starting from at least about 2-fold (e.g., 3-fold, 4-fold, 5-fold, 6-fold, etc.).

The genetic modification of a microorganism to provide increased or decreased activity (including expression, specific activity, in vivo activity, etc.) preferably affects the activity of a chitin and/or chitosan biosynthetic pathway in the microorganism, whether the pathway is endogenous and genetically modified, endogenous with the introduction of one or more recombinant nucleic acid molecules into the organism, or provided completely by recombinant technology. According to the present invention, to “affect the activity of a chitin and/or chitosan biosynthetic pathway” includes any genetic modification that causes any detectable or measurable change or modification in the chitin and/or chitosan biosynthetic pathway expressed by the organism as compared to in the absence of the genetic modification. A detectable change or modification in the chitin and/or chitosan biosynthetic pathway can include, but is not limited to, a detectable change in the production of at least one product in the chitin and/or chitosan biosynthetic pathway, or a detectable change in the production of chitin and/or chitosan by the microorganism.

In one embodiment of the present invention, a genetic modification includes a modification of a nucleic acid sequence encoding a particular enzyme or other protein as described herein. Such a modification can be to the endogenous enzyme or protein, whereby a microorganism that naturally contains such a protein is genetically modified by, for example, classical mutagenesis and selection techniques and/or molecular genetic techniques, include genetic engineering techniques. Genetic engineering techniques can include, for example, using a targeting recombinant vector to delete a portion of an endogenous gene or to replace a portion of an endogenous gene with a heterologous sequence, such as a sequence encoding an improved enzyme or other protein or a different promoter that increases the expression of the endogenous enzyme or other protein. Genetic engineering techniques can also include overexpression of a gene using recombinant technology.

For example, a non-native promoter can be introduced upstream of at least one gene encoding an enzyme or other protein of interest in the amino sugar metabolic pathway described herein. Preferably the 5′ upstream sequence of a endogenous gene is replaced by a constitutive promoter, an inducible promoter, or a promoter with optimal expression under the growth conditions used. This method is especially useful when the endogenous gene is not active or is not sufficiently active under the growth conditions used.

In another aspect of this embodiment of the invention, the genetic modification can include the introduction of a recombinant nucleic acid molecule encoding an enzyme or protein of interest into a host. The host can include: (1) a host cell that does not express the particular enzyme or protein, or (2) a host cell that does express the particular enzyme or protein, wherein the introduced recombinant nucleic acid molecule changes or enhances the activity of the enzyme or other protein in the microorganism. The present invention intends to encompass any genetically modified microorganism, wherein the microorganism comprises at least one modification suitable for a fermentation process to produce chitin and/or chitosan according to the present invention.

A genetically modified microorganism can be modified by recombinant technology, such as by introduction of an isolated nucleic acid molecule into a microorganism. For example, a genetically modified microorganism can be transfected with a recombinant nucleic acid molecule encoding a protein of interest, such as a protein for which increased expression is desired. The transfected nucleic acid molecule can remain extrachromosomal or can integrate into one or more sites within a chromosome of the transfected (i.e., recombinant) host cell in such a manner that its ability to be expressed is retained. Preferably, once a host cell of the present invention is transfected with a nucleic acid molecule, the nucleic acid molecule is integrated into the host cell genome. A significant advantage of integration is that the nucleic acid molecule is stably maintained in the cell. In a preferred embodiment, the integrated nucleic acid molecule is operatively linked to a transcription control sequence (described below) that can be induced to control expression of the nucleic acid molecule.

A nucleic acid molecule can be integrated into the genome of the host cell either by random or targeted integration. Such methods of integration are known in the art. A genetically modified microorganism can also be produced by introducing nucleic acid molecules into a recipient cell genome by a method such as by using a transducing bacteriophage. The use of recombinant technology and transducing bacteriophage technology to produce several different genetically modified microorganism of the present invention is known in the art.

It is to be understood that the present invention discloses a method comprising the use of a microorganism with an ability to produce commercially useful amounts of chitin and/or chitosan in a fermentation process (i.e., preferably an enhanced ability to produce chitin and/or chitosan compared to a wild-type microorganism cultured under the same conditions). As used herein, a fermentation process is a process of culturing cells, such as microorganisms, in a container, bioreactor, fermenter, or other suitable culture chamber, in order to produce a product from the cells (i.e., the cells produce a product during the culture process). The product is typically a product useful for experimental or commercial purposes. The fermentation method of the present invention is achieved by the genetic modification of one or more genes encoding a protein involved in a metabolic pathway discussed herein which results in the production (expression) of a protein having an altered (e.g., increased or decreased) function as compared to the corresponding wild-type protein. Such an altered function enhances the ability of the genetically engineered microorganism to produce chitin and/or chitosan. It will be appreciated by those of skill in the art that production of genetically modified microorganisms having a particular altered function as described herein, such as by the specific selection techniques described in the Examples, can produce many organisms meeting the given functional requirement, albeit by virtue of a variety of different genetic modifications. For example, different random nucleotide deletions and/or substitutions in a given nucleic acid sequence may all give rise to the same phenotypic result (e.g., decreased action of the protein encoded by the sequence). The present invention contemplates any such genetic modification which results in the production of a microorganism having the characteristics set forth herein.

In one aspect of the invention, a genetically modified microorganism useful in a fermentation method produces at least about 50% more chitin and/or chitosan, more preferably 100% more chitin and/or chitosan, and more preferably about 2 fold more chitin or chitosan, and more preferably, at least about 2.5-fold more chitin and/or chitosan, and preferably at least about 5-fold, and more preferably at least about 10-fold, and more preferably at least about 25-fold, and more preferably at least about 50-fold, and even more preferably at least about 100-fold, and even more preferably, at least about 200-fold, and even more preferably, at least about 300-fold or higher, including any fold increase between at least 2-fold and at least 300-fold, in 0.5 integer increments (i.e., at least 3-fold, at least 3.5-fold, at least 4-fold, etc.), more chitin and/or chitosan than a wild-type (i.e., non-modified, naturally occurring) microorganism of the same species (and preferably strain) cultured under the same conditions or equivalent conditions as the genetically modified microorganism. Microorganisms having such characteristics are described in the Examples section.

According to the present invention, reference to a particular enzyme or other protein herein refers to any protein that has at least one biological activity of the wild-type reference protein, including full-length proteins, fusion proteins, or any homologue of a naturally occurring protein (including natural allelic variants, fragments, related proteins from different organisms and synthetically or artificially derived variants (homologues)). A homologue (mutant, variant, modified form) of a reference protein includes proteins which differ from the naturally occurring reference protein in that at least one or a few, but not limited to one or a few, amino acids have been deleted (e.g., a truncated version of the protein, such as a peptide or fragment), inserted, inverted, substituted and/or derivatized (e.g., by glycosylation, phosphorylation, acetylation, myristoylation, prenylation, palmitation, amidation and/or addition of glycosylphosphatidyl inositol). One preferred homologue is a biologically active fragment of a naturally occurring protein. Other preferred homologues of naturally occurring proteins useful in the present invention are described in detail below. Therefore, an isolated nucleic acid molecule of the present invention can encode the translation product of any specified protein open reading frame, domain, biologically active fragment thereof, or any homologue of a naturally occurring protein or domain which has biological activity.

An isolated protein, according to the present invention, is a protein that has been removed from its natural milieu (i.e., that has been subject to human manipulation) and can include purified proteins, partially purified proteins, recombinantly produced proteins, and synthetically produced proteins, for example. Several recombinantly produced proteins are described in the Examples section. As such, “isolated” does not reflect the extent to which the protein has been purified. In addition, and by way of example by referencing a hypothetical protein called “protein X” (i.e., any enzyme or protein of used in the invention can be substituted for the term), an “E. coli protein X” refers to a protein X (including a homologue of a naturally occurring protein X) from E. coli or to a protein X that has been otherwise produced from the knowledge of the structure (e.g., sequence) and perhaps the function of a naturally occurring protein X from E. coli. In other words, an E. coli protein X includes any protein X that has substantially similar structure and function of a naturally occurring protein X from E. coli or that is a biologically active (i.e., has biological activity) homologue of a naturally occurring protein X from E. coli as described in detail herein. As such, an E. coli protein X can include purified, partially purified, recombinant, mutated/modified and synthetic proteins. This discussion applies similarly to protein X from other microorganisms as disclosed herein.

Homologues can be the result of natural allelic variation or natural mutation. A naturally occurring allelic variant of a nucleic acid encoding a protein is a gene that occurs at essentially the same locus (or loci) in the genome as the gene which encodes such protein, but which, due to natural variations caused by, for example, mutation or recombination, has a similar but not identical sequence. Allelic variants typically encode proteins having similar activity to that of the protein encoded by the gene to which they are being compared. One class of allelic variants can encode the same protein but have different nucleic acid sequences due to the degeneracy of the genetic code. Allelic variants can also comprise alterations in the 5′ or 3′ untranslated regions of the gene (e.g., in regulatory control regions). Allelic variants are well known to those skilled in the art.

Homologues can be produced using techniques known in the art for the production of proteins including, but not limited to, direct modifications to the isolated, naturally occurring protein, direct protein synthesis, or modifications to the nucleic acid sequence encoding the protein using, for example, classic or recombinant DNA techniques to effect random or targeted mutagenesis.

Modifications in homologues, as compared to the wild-type protein, agonize, antagonize, or do not substantially change, the basic biological activity of the homologue as compared to the naturally occurring protein. In general, the biological activity or biological action of a protein refers to any function(s) exhibited or performed by the protein that is ascribed to the naturally occurring form of the protein as measured or observed in vivo (i.e., in the natural physiological environment of the protein) or in vitro (i.e., under laboratory conditions). The biological activity of the enzymes and proteins used herein have been described in detail above. Modifications of a protein, such as in a homologue, may result in proteins having the same level of biological activity as the naturally occurring protein, or in proteins having decreased or increased biological activity as compared to the naturally occurring protein. Modifications which result in a decrease in expression or a decrease in the activity of the protein, can be referred to as inactivation (complete or partial), down-regulation, or decreased action of a protein. Similarly, modifications which result in an increase in expression or an increase in the activity of the protein, can be referred to as amplification, overproduction, activation, enhancement, up-regulation or increased action of a protein. A functional subunit, homologue, or fragment of a given protein is preferably capable of performing substantially the same (e.g., at least qualitatively the same) biological function of the native protein (i.e., has biological activity). It is noted that a functional subunit, fragment or other homologue of a protein is not necessarily required to have the same level of biological activity as the reference or wild-type protein in order to be considered to have the biological activity of the reference or wild-type protein (i.e., a qualitative similarity is sufficient). In one embodiment, it is preferred that modifications in homologues as compared to the wild-type protein do not substantially decrease the basic biological activity of the protein as compared to the naturally occurring protein. Increased biological activity (e.g., increased enzyme activity) may be desirable in a homologue. Homologues may also have differences in characteristics other than the functional, or enzymatic, activity of the protein as compared to the naturally occurring form, such as a decreased sensitivity to inhibition by certain compounds as compared to the naturally occurring protein.

According to the present invention, an isolated protein, including a biologically active homologue or fragment thereof, has at least one characteristic of biological activity of the wild-type, or naturally occurring protein. Methods of detecting and measuring protein expression and biological activity include, but are not limited to, measurement of transcription of the protein, measurement of translation of the protein, measurement of cellular localization of the protein, measurement of binding or association of the protein with another protein, measurement of binding or association of the gene encoding the protein regulatory sequences to a protein or other nucleic acid, measurement of an increase, decrease or induction of biological activity of the protein in a cell that expresses the protein.

Methods to measure protein expression levels of a protein according to the invention include, but are not limited to: western blotting, immunocytochemistry, flow cytometry or other immunologic-based assays; assays based on a property of the protein including but not limited to, ligand binding, enzyme activity or interaction with other protein partners. Binding assays are also well known in the art. For example, a BIAcore machine can be used to determine the binding constant of a complex between two proteins. The dissociation constant for the complex can be determined by monitoring changes in the refractive index with respect to time as buffer is passed over the chip (O'Shannessy et al. Anal. Biochem. 212:457-468 (1993); Schuster et al., Nature 365:343-347 (1993)). Other suitable assays for measuring the binding of one protein to another include, for example, immunoassays such as enzyme linked immunoabsorbent assays (ELISA) and radioimmunoassays (RIA), or determination of binding by monitoring the change in the spectroscopic or optical properties of the proteins through fluorescence, UV absorption, circular dichroism, or nuclear magnetic resonance (NMR). Assays for measuring the enzymatic activity of a protein used in the invention are well known in the art and many are described in the Examples section.

Many of the enzymes and proteins involved in the metabolic pathways described herein and which represent desirable targets for modification and use in the fermentation processes described herein have been described above in terms of function and amino acid sequence (and nucleic acid sequence encoding the same) of representative wild-type or mutant proteins. In one embodiment of the invention, homologues of a given protein (which can include related proteins from other organisms or modified forms of the given protein) are encompassed for use in a genetically modified organism of the invention. Homologues of a proteins encompassed by the present invention can comprise an amino acid sequence that is at least about 35% identical, and more preferably at least about 40% identical, and more preferably at least about 45% identical, and more preferably at least about 50% identical, and more preferably at least about 55% identical, and more preferably at least about 60% identical, and more preferably at least about 65% identical, and more preferably at least about 70% identical, and more preferably at least about 75% identical, and more preferably at least about 80% identical, and more preferably at least about 85% identical, and more preferably at least about 90% identical, and more preferably at least about 95% identical, and more preferably at least about 96% identical, and more preferably at least about 97% identical, and more preferably at least about 98% identical, and more preferably at least about 99% identical, or any percent identity between 35% and 99%, in whole integers (i.e., 36%, 37%, etc.) to an amino acid sequence disclosed herein that represents the amino acid sequence of an enzyme or protein that can be modified or overexpressed according to the invention. Preferably, the amino acid sequence of the homologue has a biological activity of the wild-type or reference protein.

As used herein, unless otherwise specified, reference to a percent (%) identity refers to an evaluation of homology which is performed using: (1) a BLAST 2.0 Basic BLAST homology search using blastp for amino acid searches and blastn for nucleic acid searches with standard default parameters, wherein the query sequence is filtered for low complexity regions by default (described in Altschul, S. F., Madden, T. L., Schaffer, A. A., Zhang, J., Zhang, Z., Miller, W. & Lipman, D. J. (1997) “Gapped BLAST and PSI-BLAST: a new generation of protein database search programs.” Nucleic Acids Res. 25:3389-3402, incorporated herein by reference in its entirety); (2) a BLAST 2 alignment (using the parameters described below); (3) and/or PSI-BLAST with the standard default parameters (Position-Specific Iterated BLAST. It is noted that due to some differences in the standard parameters between BLAST 2.0 Basic BLAST and BLAST 2, two specific sequences might be recognized as having significant homology using the BLAST 2 program, whereas a search performed in BLAST 2.0 Basic BLAST using one of the sequences as the query sequence may not identify the second sequence in the top matches. In addition, PSI-BLAST provides an automated, easy-to-use version of a “profile” search, which is a sensitive way to look for sequence homologues. The program first performs a gapped BLAST database search. The PSI-BLAST program uses the information from any significant alignments returned to construct a position-specific score matrix, which replaces the query sequence for the next round of database searching. Therefore, it is to be understood that percent identity can be determined by using any one of these programs.

Two specific sequences can be aligned to one another using BLAST 2 sequence as described in Tatusova and Madden, (1999), “Blast 2 sequences—a new tool for comparing protein and nucleotide sequences”, FEMS Microbiol Lett. 174:247-250, incorporated herein by reference in its entirety. BLAST 2 sequence alignment is performed in blastp or blastn using the BLAST 2.0 algorithm to perform a Gapped BLAST search (BLAST 2.0) between the two sequences allowing for the introduction of gaps (deletions and insertions) in the resulting alignment. For purposes of clarity herein, a BLAST 2 sequence alignment is performed using the standard default parameters as follows.

For blastn, using 0 BLOSUM62 matrix:

Reward for match=1
Penalty for mismatch=−2
Open gap (5) and extension gap (2) penalties
gap x_dropoff (50) expect (10) word size (11) filter (on)
   For blastp, using 0 BLOSUM62 matrix:
Open gap (11) and extension gap (1) penalties
gap x_dropoff (50) expect (10) word size (3) filter (on).

A protein referenced and/or used in the present invention can also include proteins having an amino acid sequence comprising at least 30 contiguous amino acid residues of the amino acid sequence of the reference protein (i.e., 30 contiguous amino acid residues having 100% identity with 30 contiguous amino acids of either of the above-identified sequences). In a preferred embodiment, a protein referenced and/or used in the present invention includes proteins having amino acid sequences comprising at least 50, and more preferably at least 75, and more preferably at least 100, and more preferably at least 115, and more preferably at least 130, and more preferably at least 150, and more preferably at least 200, and more preferably, at least 250, and more preferably, at least 300, and more preferably, at least 350 contiguous amino acid residues of the amino acid sequence of the reference protein. In one embodiment, such a protein has a biological activity of the reference protein. According to the present invention, the term “contiguous” or “consecutive”, with regard to nucleic acid or amino acid sequences described herein, means to be connected in an unbroken sequence. For example, for a first sequence to comprise 30 contiguous (or consecutive) amino acids of a second sequence, means that the first sequence includes an unbroken sequence of 30 amino acid residues that is 100% identical to an unbroken sequence of 30 amino acid residues in the second sequence. Similarly, for a first sequence to have “100% identity” with a second sequence means that the first sequence exactly matches the second sequence with no gaps between nucleotides or amino acids.

In another embodiment, a protein referenced or used in the present invention, including a homologue, includes a protein having an amino acid sequence that is sufficiently similar to the naturally occurring protein amino acid sequence that a nucleic acid sequence encoding the homologue is capable of hybridizing under moderate, high, or very high stringency conditions (described below) to (i.e., with) a nucleic acid molecule encoding the naturally occurring protein (i.e., to the complement of the nucleic acid strand encoding the naturally occurring protein). Preferably, a given homologue is encoded by a nucleic acid sequence that hybridizes under moderate, high or very high stringency conditions to the complement of a nucleic acid sequence that encodes the wild-type or reference protein.

A nucleic acid sequence complement of reference nucleic acid sequence refers to the nucleic acid sequence of the nucleic acid strand that is complementary to the strand which encodes a protein. It will be appreciated that a double stranded DNA which encodes a given amino acid sequence comprises a single strand DNA and its complementary strand having a sequence that is a complement to the single strand DNA. As such, nucleic acid molecules of the present invention can be either double-stranded or single-stranded, and include those nucleic acid molecules that form stable hybrids under stringent hybridization conditions with a nucleic acid sequence that encodes an amino acid sequence of a protein, and/or with the complement of the nucleic acid sequence that encodes such protein. Methods to deduce a complementary sequence are known to those skilled in the art. It should be noted that since amino acid sequencing and nucleic acid sequencing technologies are not entirely error-free, the sequences presented herein, at best, represent apparent sequences of the referenced proteins of the present invention.

As used herein, reference to hybridization conditions refers to standard hybridization conditions under which nucleic acid molecules are used to identify similar nucleic acid molecules. Such standard conditions are disclosed, for example, in Sambrook et al., Molecular Cloning: A Laboratory Manual, Cold Spring Harbor Labs Press, 1989. Sambrook et al., ibid., is incorporated by reference herein in its entirety (see specifically, pages 9.31-9.62). In addition, formulae to calculate the appropriate hybridization and wash conditions to achieve hybridization permitting varying degrees of mismatch of nucleotides are disclosed, for example, in Meinkoth et al., 1984, Anal. Biochem. 138, 267-284; Meinkoth et al., ibid., is incorporated by reference herein in its entirety.

More particularly, moderate stringency hybridization and washing conditions, as referred to herein, refer to conditions which permit isolation of nucleic acid molecules having at least about 70% nucleic acid sequence identity with the nucleic acid molecule being used to probe in the hybridization reaction (i.e., conditions permitting about 30% or less mismatch of nucleotides). High stringency hybridization and washing conditions, as referred to herein, refer to conditions which permit isolation of nucleic acid molecules having at least about 80% nucleic acid sequence identity with the nucleic acid molecule being used to probe in the hybridization reaction (i.e., conditions permitting about 20% or less mismatch of nucleotides). Very high stringency hybridization and washing conditions, as referred to herein, refer to conditions which permit isolation of nucleic acid molecules having at least about 90% nucleic acid sequence identity with the nucleic acid molecule being used to probe in the hybridization reaction (i.e., conditions permitting about 10% or less mismatch of nucleotides). As discussed above, one of skill in the art can use the formulae in Meinkoth et al., ibid to calculate the appropriate hybridization and wash conditions to achieve these particular levels of nucleotide mismatch. Such conditions will vary, depending on whether DNA:RNA or DNA:DNA hybrids are being formed. Calculated melting temperatures for DNA:DNA hybrids are 10° C. less than for DNA:RNA hybrids. In particular embodiments, stringent hybridization conditions for DNA:DNA hybrids include hybridization at an ionic strength of 6× SSC (0.9 M Na⁺) at a temperature of between about 20° C. and about 35° C. (lower stringency), more preferably, between about 28° C. and about 40° C. (more stringent), and even more preferably, between about 35° C. and about 45° C. (even more stringent), with appropriate wash conditions. In particular embodiments, stringent hybridization conditions for DNA:RNA hybrids include hybridization at an ionic strength of 6×SSC (0.9 M Na⁺) at a temperature of between about 30° C. and about 45° C., more preferably, between about 38° C. and about 50° C., and even more preferably, between about 45° C. and about 55° C., with similarly stringent wash conditions. These values are based on calculations of a melting temperature for molecules larger than about 100 nucleotides, 0% formamide and a G+C content of about 40%. Alternatively, Tₘ can be calculated empirically as set forth in Sambrook et al., supra, pages 9.31 to 9.62. In general, the wash conditions should be as stringent as possible, and should be appropriate for the chosen hybridization conditions. For example, hybridization conditions can include a combination of salt and temperature conditions that are approximately 20-25° C. below the calculated Tₘ of a particular hybrid, and wash conditions typically include a combination of salt and temperature conditions that are approximately 12-20° C. below the calculated Tₘ of the particular hybrid. One example of hybridization conditions suitable for use with DNA:DNA hybrids includes a 2-24 hour hybridization in 6×SSC (50% formamide) at about 42° C., followed by washing steps that include one or more washes at room temperature in about 2×SSC, followed by additional washes at higher temperatures and lower ionic strength (e.g., at least one wash as about 37° C. in about 0.1×-0.5× SSC, followed by at least one wash at about 68° C. in about 0.1×-0.5× SSC).

The minimum size of a protein and/or homologue of the present invention is, in one aspect, a size sufficient to have the desired biological activity of the protein. In another embodiment, a protein of the present invention is at least 30 amino acids long, and more preferably, at least about 50, and more preferably at least 75, and more preferably at least 100, and more preferably at least 115, and more preferably at least 130, and more preferably at least 150, and more preferably at least 200, and more preferably, at least 250, and more preferably, at least 300, and more preferably, at least 350 amino acids long. There is no limit, other than a practical limit, on the maximum size of such a protein in that the protein can include a portion of a given protein or a full-length protein, plus additional sequence (e.g., a fusion protein sequence), if desired. Suitable fusion segments for use with the present invention include, but are not limited to, segments that can: enhance a protein's stability; provide other desirable biological activity (e.g., a second enzyme function); and/or assist with the purification of a protein (e.g., by affinity chromatography).

In one embodiment of the present invention, any of the amino acid sequences described herein can be produced with from at least one, and up to about 20, additional heterologous amino acids flanking each of the C- and/or N-terminal ends of the specified amino acid sequence. The resulting protein or polypeptide can be referred to as “consisting essentially of” the specified amino acid sequence. According to the present invention, the heterologous amino acids are a sequence of amino acids that are not naturally found (i.e., not found in nature, in vivo) flanking the specified amino acid sequence, or that are not related to the function of the specified amino acid sequence, or that would not be encoded by the nucleotides that flank the naturally occurring nucleic acid sequence encoding the specified amino acid sequence as it occurs in the gene, if such nucleotides in the naturally occurring sequence were translated using standard codon usage for the organism from which the given amino acid sequence is derived. Similarly, the phrase “consisting essentially of”, when used with reference to a nucleic acid sequence herein, refers to a nucleic acid sequence encoding a specified amino acid sequence that can be flanked by from at least one, and up to as many as about 60, additional heterologous nucleotides at each of the 5′ and/or the 3′ end of the nucleic acid sequence encoding the specified amino acid sequence. The heterologous nucleotides are not naturally found (i.e., not found in nature, in vivo) flanking the nucleic acid sequence encoding the specified amino acid sequence as it occurs in the natural gene or do not encode a protein that imparts any additional function to the protein or changes the function of the protein having the specified amino acid sequence.

Embodiments of the present invention include the use and/or manipulation of nucleic acid molecules that encode enzymes or other proteins in the metabolic pathways described herein. A nucleic acid molecule of the present invention includes a nucleic acid molecule comprising, consisting essentially of, or consisting of, a nucleic acid sequence encoding any of the enzymes or other proteins described herein.

In accordance with the present invention, an isolated nucleic acid molecule is a nucleic acid molecule that has been removed from its natural milieu (i.e., that has been subject to human manipulation), its natural milieu being the genome or chromosome in which the nucleic acid molecule is found in nature. As such, “isolated” does not necessarily reflect the extent to which the nucleic acid molecule has been purified, but indicates that the molecule does not include an entire genome or an entire chromosome in which the nucleic acid molecule is found in nature. An isolated nucleic acid molecule can include a gene. An isolated nucleic acid molecule that includes a gene is not a fragment of a chromosome that includes such gene, but rather includes the coding region and regulatory regions associated with the gene, but no additional genes naturally found on the same chromosome. An isolated nucleic acid molecule can also include a specified nucleic acid sequence flanked by (i.e., at the 5′ and/or the 3′ end of the sequence) additional nucleic acids that do not normally flank the specified nucleic acid sequence in nature (i.e., are heterologous sequences). Isolated nucleic acid molecules can include DNA, RNA (e.g., mRNA), or derivatives of either DNA or RNA (e.g., cDNA). Although the phrase “nucleic acid molecule” primarily refers to the physical nucleic acid molecule and the phrase “nucleic acid sequence” primarily refers to the sequence of nucleotides on the nucleic acid molecule, the two phrases can be used interchangeably, especially with respect to a nucleic acid molecule, or a nucleic acid sequence, being capable of encoding a protein.

Preferably, an isolated nucleic acid molecule of the present invention is produced using recombinant DNA technology (e.g., polymerase chain reaction (PCR) amplification, cloning) or chemical synthesis. Isolated nucleic acid molecules include natural nucleic acid molecules and homologues thereof, including, but not limited to, natural allelic variants and modified nucleic acid molecules in which nucleotides have been inserted, deleted, substituted, and/or inverted in such a manner that such modifications provide the desired effect on protein biological activity. Allelic variants and protein homologues (e.g., proteins encoded by nucleic acid homologues) have been discussed in detail above.

A nucleic acid molecule homologue can be produced using a number of methods known to those skilled in the art (see, for example, Sambrook et al., ibid.). For example, nucleic acid molecules can be modified using a variety of techniques including, but not limited to, classical mutagenesis techniques and recombinant DNA techniques, such as site-directed mutagenesis, chemical treatment of a nucleic acid molecule to induce mutations, restriction enzyme cleavage of a nucleic acid fragment, ligation of nucleic acid fragments, PCR amplification and/or mutagenesis of selected regions of a nucleic acid sequence, synthesis of oligonucleotide mixtures and ligation of mixture groups to “build” a mixture of nucleic acid molecules and combinations thereof. Nucleic acid molecule homologues can be selected from a mixture of modified nucleic acids by screening for the function of the protein encoded by the nucleic acid and/or by hybridization with a wild-type gene.

The minimum size of a nucleic acid molecule of the present invention is a size sufficient to encode a protein having the desired biological activity, or sufficient to form a probe or oligonucleotide primer that is capable of forming a stable hybrid with the complementary sequence of a nucleic acid molecule encoding the natural protein (e.g., under moderate, high or very high stringency conditions, and preferably under very high stringency conditions). As such, the size of a nucleic acid molecule of the present invention can be dependent on nucleic acid composition and percent homology or identity between the nucleic acid molecule and complementary sequence as well as upon hybridization conditions per se (e.g., temperature, salt concentration, and formamide concentration). The minimal size of a nucleic acid molecule that is used as an oligonucleotide primer or as a probe is typically at least about 12 to about 15 nucleotides in length if the nucleic acid molecules are GC-rich and at least about 15 to about 18 bases in length if they are AT-rich. There is no limit, other than a practical limit, on the maximal size of a nucleic acid molecule of the present invention, in that the nucleic acid molecule can include a portion of a protein encoding sequence, a nucleic acid sequence encoding a full-length protein (including a complete gene).

Knowing the nucleic acid sequences of certain nucleic acid molecules of the present invention, and particularly any of the nucleic acid molecules described in detail herein, allows one skilled in the art to, for example, (a) make copies of those nucleic acid molecules and/or (b) obtain nucleic acid molecules including at least a portion of such nucleic acid molecules (e.g., nucleic acid molecules including full-length genes, full-length coding regions, regulatory control sequences, truncated coding regions). Such nucleic acid molecules can be obtained in a variety of ways including traditional cloning techniques using oligonucleotide probes of to screen appropriate libraries or DNA and PCR amplification of appropriate libraries or DNA using oligonucleotide primers. Preferred libraries to screen or from which to amplify nucleic acid molecule include bacterial and yeast genomic DNA libraries, and in particular, Escherichia coli genomic DNA libraries. Techniques to clone and amplify genes are disclosed, for example, in Sambrook et al., ibid.

Another embodiment of the present invention includes a recombinant nucleic acid molecule comprising a recombinant vector and a nucleic acid molecule comprising a nucleic acid sequence encoding an amino acid sequence having a biological activity of any of the enzymes or other proteins in a metabolic pathway as described herein. According to the present invention, a recombinant vector is an engineered (i.e., artificially produced) nucleic acid molecule that is used as a tool for manipulating a nucleic acid sequence of choice and for introducing such a nucleic acid sequence into a host cell. The recombinant vector is therefore suitable for use in cloning, sequencing, and/or otherwise manipulating the nucleic acid sequence of choice, such as by expressing and/or delivering the nucleic acid sequence of choice into a host cell to form a recombinant cell. Such a vector typically contains heterologous nucleic acid sequences, that is nucleic acid sequences that are not naturally found adjacent to nucleic acid sequence to be cloned or delivered, although the vector can also contain regulatory nucleic acid sequences (e.g., promoters, untranslated regions) which are naturally found adjacent to nucleic acid molecules of the present invention or which are useful for expression of the nucleic acid molecules of the present invention (discussed in detail below). The vector can be either RNA or DNA, either prokaryotic or eukaryotic, and typically is a plasmid. The vector can be maintained as an extrachromosomal element (e.g., a plasmid) or it can be integrated into the chromosome of a recombinant organism (e.g., a microbe or a plant). The entire vector can remain in place within a host cell, or under certain conditions, the plasmid DNA can be deleted, leaving behind the nucleic acid molecule of the present invention. The integrated nucleic acid molecule can be under chromosomal promoter control, under native or plasmid promoter control, or under a combination of several promoter controls. Single or multiple copies of the nucleic acid molecule can be integrated into the chromosome. A recombinant vector of the present invention can contain at least one selectable marker.

In one embodiment, a recombinant vector used in a recombinant nucleic acid molecule of the present invention is an expression vector. As used herein, the phrase “expression vector” is used to refer to a vector that is suitable for production of an encoded product (e.g., a protein of interest). In this embodiment, a nucleic acid sequence encoding the product to be produced is inserted into the recombinant vector to produce a recombinant nucleic acid molecule. The nucleic acid sequence encoding the protein to be produced is inserted into the vector in a manner that operatively links the nucleic acid sequence to regulatory sequences in the vector which enable the transcription and translation of the nucleic acid sequence within the recombinant host cell.

In another embodiment, a recombinant vector used in a recombinant nucleic acid molecule of the present invention is a targeting vector. As used herein, the phrase “targeting vector” is used to refer to a vector that is used to deliver a particular nucleic acid molecule into a recombinant host cell, wherein the nucleic acid molecule is used to delete or inactivate an endogenous gene within the host cell or microorganism (i.e., used for targeted gene disruption or knock-out technology). Such a vector may also be known in the art as a “knock-out” vector. In one aspect of this embodiment, a portion of the vector, but more typically, the nucleic acid molecule inserted into the vector (i.e., the insert), has a nucleic acid sequence that is homologous to a nucleic acid sequence of a target gene in the host cell (i.e., a gene which is targeted to be deleted or inactivated). The nucleic acid sequence of the vector insert is designed to bind to the target gene such that the target gene and the insert undergo homologous recombination, whereby the endogenous target gene is deleted, inactivated or attenuated (i.e., by at least a portion of the endogenous target gene being mutated or deleted).

Typically, a recombinant nucleic acid molecule includes at least one nucleic acid molecule of the present invention operatively linked to one or more expression control sequences, including transcription control sequences and translation control sequences. As used herein, the phrase “recombinant molecule” or “recombinant nucleic acid molecule” primarily refers to a nucleic acid molecule or nucleic acid sequence operatively linked to an expression control sequence, but can be used interchangeably with the phrase “nucleic acid molecule”, when such nucleic acid molecule is a recombinant molecule as discussed herein. According to the present invention, the phrase “operatively linked” refers to linking a nucleic acid molecule to an expression control sequence (e.g., a transcription control sequence and/or a translation control sequence) in a manner such that the molecule is able to be expressed when transfected (i.e., transformed, transduced, transfected, conjugated or conduced) into a host cell. Transcription control sequences are sequences which control the initiation, elongation, or termination of transcription. Particularly important transcription control sequences are those which control transcription initiation, such as promoter, enhancer, operator and repressor sequences. Suitable transcription control sequences include any transcription control sequence that can function in a host cell or organism into which the recombinant nucleic acid molecule is to be introduced.

Recombinant nucleic acid molecules of the present invention can also contain additional regulatory sequences, such as translation regulatory sequences, origins of replication, and other regulatory sequences that are compatible with the recombinant cell. In one embodiment, a recombinant molecule of the present invention, including those which are integrated into the host cell chromosome, also contains secretory signals (i.e., signal segment nucleic acid sequences) to enable an expressed protein to be secreted from the cell that produces the protein. Suitable signal segments include a signal segment that is naturally associated with the protein to be expressed or any heterologous signal segment capable of directing the secretion of the protein according to the present invention. In another embodiment, a recombinant molecule of the present invention comprises a leader sequence to enable an expressed protein to be delivered to and inserted into the membrane of a host cell. Suitable leader sequences include a leader sequence that is naturally associated with the protein, or any heterologous leader sequence capable of directing the delivery and insertion of the protein to the membrane of a cell.

It is preferred that the recombinant nucleic acid molecules comprising nucleic acid sequences encoding various enzymes and proteins described herein (including homologues thereof) be cloned under control of an artificial promoter. The promoter can be any suitable promoter that will provide a level of gene expression required to maintain a sufficient level of the encoded protein in the production organism. Suitable promoters can be promoters inducible by different chemicals (such as lactose, galactose, maltose and salt) or changes of growth conditions (such as temperature). Use of inducible promoter can lead to an optimal performance of gene expression and fermentation process. Preferred promoters can also be constitutive promoters, since the need for addition of expensive inducers is therefore obviated. Such promoters include normally inducible promoter systems that have been made functionally constitutive or “leaky” by genetic modification, such as by using a weaker, mutant repressor gene. In one embodiment, the preferred promoters include, but are not limited to, ADH1 promoter, PGK promoter, GAL1 promoter, GAL promoter or gpdA promoter from yeast or filamentous fungi such as A. niger as described in the Examples section. Exogenous promoters can also be used to modify the expression of endogenous genes in a host cell. The gene dosage (copy number) can be varied according to the requirements for maximum product formation. In one embodiment, the recombinant genes are integrated into the host genome.

It may be appreciated by one skilled in the art that use of recombinant DNA technologies can improve expression of transformed nucleic acid molecules by manipulating, for example, the number of copies of the nucleic acid molecules within a host cell, the efficiency with which those nucleic acid molecules are transcribed, the efficiency with which the resultant transcripts are translated, and the efficiency of post-translational modifications. Recombinant techniques useful for increasing the expression of nucleic acid molecules of the present invention include, but are not limited to, operatively linking nucleic acid molecules to high-copy number plasmids, integration of the nucleic acid molecules into the host cell chromosome, addition of vector stability sequences to plasmids, substitutions or modifications of transcription control signals (e.g., promoters, operators, enhancers), substitutions or modifications of translational control signals, modification of nucleic acid molecules of the present invention to correspond to the codon usage of the host cell, deletion of sequences that destabilize transcripts, and use of control signals that temporally separate recombinant cell growth from recombinant enzyme production during fermentation. The activity of an expressed recombinant protein of the present invention may be improved by fragmenting, modifying, or derivatizing nucleic acid molecules encoding such a protein.

According to the present invention, the term “transfection” is used to refer to any method by which an exogenous nucleic acid molecule (i.e., a recombinant nucleic acid molecule) can be inserted into a cell. The term “transformation” can be used interchangeably with the term “transfection” when such term is used to refer to the introduction of nucleic acid molecules into microbial cells, such as algae, bacteria and yeast, or into plant cells. In microbial systems and plant systems, the term “transformation” is used to describe an inherited change due to the acquisition of exogenous nucleic acids by the microorganism or plant and is essentially synonymous with the term “transfection.” Therefore, transfection techniques include, but are not limited to, transformation, chemical treatment of cells, particle bombardment, electroporation, microinjection, lipofection, adsorption, infection and protoplast fusion.

A recombinant cell is preferably produced by transforming a host cell (e.g., a yeast or other fungal cell) with one or more recombinant molecules, each comprising one or more nucleic acid molecules operatively linked to an expression vector containing one or more transcription control sequences. The phrase, operatively linked, refers to insertion of a nucleic acid molecule into an expression vector in a manner such that the molecule can be expressed when transformed into a host cell. As used herein, an expression vector is a DNA or RNA vector that is capable of transforming a host cell and of effecting expression of a specified nucleic acid molecule. Preferably, the expression vector is also capable of replicating within the host cell. In the present invention, expression vectors are typically plasmids. Expression vectors of the present invention include any vectors that function (i.e., direct gene expression) in a host cell. Preferred host cells include, but are not limited to any suitable bacterium, a protist, a microalgae, a fungus, or other microbe, with fungi being particularly preferred. In one preferred embodiment, the host organism is selected from a yeast and a filamentous fungus.

For metabolic engineering to increase chitin and chitosan content, suitable genera of yeast include, but are not limited to, Saccharomyces, Schizosaccharomyces, Candida, Hansenula, Pichia, Kluyveromyces, and Phaffia. Suitable yeast species include, but are not limited to, Saccharomyces cerevisiae, Schizosaccharomyces pombe, Candida albicans, Candida Guillermondii, Hansenula polymorpha, Pichia pastoris, P. canadensis, Kluyveromyces marxianus and Phaffia rhodozyma.

For metabolic engineering to increase chitin and chitosan content, suitable fungal hosts include, but are not limited to, Ascomycetes, Zygomycetes and Deuteromycetes. Suitable genus include, but are not limited to, Aspergillus, Absidia, Gongronella, Lentinus, Mucor, Phycomyces, Rhizopus, Chrysosporium, Neurospora and Trichoderma. Suitable fungal species include, but are not limited to, Aspergillus niger, Aspergillus terrreus, A. nidulans, Absidia coerulea, Absidia repens, Absidia blakesleeana, Gongronella butleri, Lentinus endodes, Mucor rouxii, Phycomyces blakesleenaus, Rhizopus oryzae, Chrysosporium lucknowense, Neurospora crassa, N. intermedia and Trichoderm reesei.

Additional embodiments of the present invention include any of the genetically modified microorganisms described herein and microorganisms having the identifying characteristics of the microorganisms specifically identified in the Examples. Such identifying characteristics can include any or all genotypic and/or phenotypic characteristics of the microorganisms in the Examples, including their abilities to produce chitin and/or chitosan.

As noted above, in the method for production of chitin and/or chitosan of the present invention, a microorganism having a genetically modified metabolic pathway is cultured in a fermentation medium for production of chitin and/or chitosan. An appropriate, or effective, fermentation medium refers to any medium in which a genetically modified microorganism of the present invention, when cultured, is capable of producing (accumulating) chitin and/or chitosan. Such a medium is typically an aqueous medium comprising assimilable carbon, nitrogen and phosphate sources. Such a medium can also include appropriate salts, minerals, metals and other nutrients. For example, a minimal-salts medium containing glucose, fructose, lactose, glycerol or a mixture of two or more different compounds as the sole carbon source is preferably used as the fermentation medium. The use of a minimal-salts-glucose medium is the most preferred medium for the chitin and/or chitosan fermentation and it will also facilitate recovery and purification of the products. In one aspect, yeast extract is a component of the medium.

Sufficient oxygen must be added to the medium during the course of the fermentation to maintain cell growth during the initial cell growth and to maintain metabolism, and chitin and/or chitosan production. Oxygen is conveniently provided by agitation and aeration of the medium. Conventional methods, such as stirring or shaking, may be used to agitate and aerate the medium. The oxygen concentration of the medium can be monitored by conventional methods, such as with an oxygen electrode. Other sources of oxygen, such as undiluted oxygen gas and oxygen gas diluted with inert gas other than nitrogen, can be used.

Microorganisms of the present invention can be cultured in conventional fermentation bioreactors. The microorganisms can be cultured by any fermentation process which includes, but is not limited to, batch, fed-batch, cell recycle, and continuous fermentation. Preferably, microorganisms of the present invention are grown by batch or fed-batch fermentation processes.

Fermentation conditions can include culturing the microorganisms of the invention at any temperature between about 20° C. and about 40° C., in whole increments (i.e., 21° C., 22° C., etc.). It is noted that the optimum temperature for growth and chitin and/or chitosan production by a microorganism of the present invention can vary according to a variety of factors. For example, the selection of a particular promoter for expression of a recombinant nucleic acid molecule in the microorganism can affect the optimum culture temperature. One of ordinary skill in the art can readily determine the optimum growth and chitin and/or chitosan production temperature for any microorganism of the present invention using standard techniques. Culture of genetically modified microorganisms according to the present invention is described in the Examples.

In addition, suitable fermentation mediums and culture conditions for microorganisms of the present invention are described in detail in U.S. Pat. No. 6,372,457 and PCT Publication No. WO 2004/003175 A2, as well as in Berka, R. M. and C. C. Barnett. 1989 and Adams et al., 1997.

In another embodiment of the present invention, methods to collect (e.g., recover) and purify chitin and chitosan from microbial biomass produced by the methods of the present invention are included in the method of chitin or chitosan production. These methods are based on those described previously in U.S. Pat. No. 4,806,474; PCT Publication No. WO 01/68714 and other publications (e.g., Rane and Hoover, 1993; Synowiecki and Al-Khateeb, 1997; Pochanavanich and Suntomsuk. 2002). Each of these publications is incorporated herein by reference in its entirety. These methods describe sequential alkaline and acidic extraction of fungal biomass followed by recovery of the extracted chitosan by alkaline precipitation.

To “collect” a product such as chitin and/or chitosan can simply refer to collecting the biomass from the fermentation bioreactor and need not imply additional steps of separation, recovery, or purification. For example, the step of collecting can refer to removing the entire culture (i.e., the microorganism and the fermentation medium) from the bioreactor, and/or removing the microorganism containing chitin and/or chitosan from the bioreactor. The term “recovering” or “recover”, as used herein with regard to recovering chitin and/or chitosan products, refers to performing additional processing steps on the microbial biomass to obtain chitin and/or chitosan at any level of purity. These steps can be followed by further purification steps. For example, chitin and/or chitosan can be recovered from the biomass by a technique that includes, but is not limited to, the following steps: treatment of microorganism cells with a hot alkaline solution, collection and washing of the remaining solids containing chitin or chitosan, resuspension of the washed solids in an acidic solution to solubilize the chitin or chitosan, and precipitation of the chitin or chitosan. Chitin and/or chitosan are preferably recovered in substantially pure forms. As used herein, “substantially pure” refers to a purity that allows for the effective use of the chitin and/or chitosan as a compound for commercial sale. In one embodiment, the chitin and/or chitosan products are preferably separated from the production organism and other fermentation medium constituents. Methods to accomplish such separation are well known in the art and are referenced above.

Preferably, by the method of the present invention, at least about 25% of product (i.e., chitin and/or chitosan) by weight are recovered from the microbial biomass and/or collected as a dry weight of chitin and/or chitosan within the microbial biomass. More preferably, by the method of the present invention, at least about 30%, and more preferably, at least about 40%, and even more preferably, at least about 45%, and even more preferably, at least about 50%, and even more preferably, at least about 60%, and even more preferably, at least about 70% and even more preferably, at least about 75% and even more preferably at least about 80% d, even more preferably at least about 85%, even more preferably at least about 90% of product are recovered, even more preferably at least about 95% of product are recovered and even more preferably at least about 98% of product are recovered, even more preferably 100% of the product is recovered, including any whole increment between at least about 25% and 100%.

Preferably, using the method of the present invention, the microorganism produces at least about 0.5% of its total biomass by dry weight as chitin or chitosan, and more preferably, at least about 1%, and more preferably, at least about 2%, and more preferably, at least about 3%, and more preferably, at least about 4%, and more preferably, at least about 5%, and more preferably, at least about 7.5%, and more preferably, at least about 10%, and more preferably, at least about 20%, and more preferably, at least about 30%, and more preferably, a least about 40% and even higher, including any increment between at least about 0.5% and 40% or greater, in 0.5% increments (e.g., 0.5%, 1%, 1.5%, 2% . . . 21.5%, 22% . . . 39.5%, 40%, 40.5%, . . . ).

In another embodiment, using the method of the present invention, the microorganism produces at least about 0.1 gram of chitin or chitosan per liter of fermentation medium in which the biomass producing the chitin or chitosan is cultured, and preferably at least about 0.2 g/L, and more preferably at least about 0.3 g/L, and more preferably at least about 0.4 g/L, and more preferably at least about 0.5 g/L, and more preferably at least about 7.5 g/L, and more preferably at least about 10 g/L, and more preferably at least about 15 g/L, and more preferably at least about 20 g/L, and more preferably at least about 25 g/L, and more preferably at least about 50 g/L, and more preferably at least about 100 g/L, and more preferably at least about 200 g/L or higher, including any increment between about 0.1 g/L and 200 g/L or higher, in 0.1 g/L increments (e.g., 0.1 g/L, 0.2 g/L, 0.3 g/L, . . . 10 g/L, 10.1 g/L, 10.2 g/L, etc.)

### EXAMPLES

The following examples are provided for the purpose of illustration and are not intended to limit the scope of the invention.

### Example 1

The following example describes expression of different glmS or GFA1 genes to increase chitin and chitosan contents in yeast.

New yeast strains will be developed to elevate levels of chitin and chitosan by overexpressing genes involved in chitin and chitosan biosynthesis. A key enzyme in this pathway is glutamine:fructose-6-phosphate amidotransferase. In E. coli, the glmS gene encodes this enzyme, the coding region of which is represented herein as SEQ ID NO:22, which encodes the amino acid sequence of SEQ ID NO:23. Different mutant versions of this gene, such as glmS*54 that code for an enzyme resistant to feedback inhibition by glucosamine-6-phosphate, the nucleic acid sequence for which is represented herein by SEQ ID NO:24, which encodes the amino acid sequence of SEQ ID NO:25, were described in the U.S. Pat. No. 6,372,457, supra. Additional mutant versions of the glmS gene were described in PCT Publication No. WO 2004/003175, supra. As a first step toward metabolic engineering of yeast for chitin and chitosan production, the E. coli glmS*54 gene will be expressed in S. cerevisiae. This was accomplished by cloning the glmS*54 open reading frame sequence into a yeast vector such that its expression is controlled by a strong yeast promoter and transcription terminator sequence. Strong yeast promoters, such as the ADH1 promoter, the TDH2 promoter, and the PGK promoter can be used for this purpose. The expression cassette can be carried on a free-replicating plasmid or integrated into the yeast chromosome. To further increase chitin and chitosan content, other chitin/chitosan biosynthetic enzymes can be engineered and/or overexpressed for increased activity in S. cerevisiae. Open reading frames from chitin and chitosan biosynthetic genes of S. cerevisiae and other organisms can be used for this purpose. Genes from S. cerevisiae that are currently known and that can be used for this purpose include, but are not limited to, GNA1 (SEQ ID NO:32 and SEQ ID NO:33), PCMI/AGM1 (nucleotide sequence X75816, amino acid sequence CAA53452.1), UAP1 (nucleotide sequence included in NC_{—}001136, amino acid sequence NP_{—}010180.1), CHS1 to CHS7 (P08004, P14180, P29465, NP_{—}009492, NP_{—}013434, NP_{—}012436 and NP_{—}012011, respectively), YEA4 (NP_{—}010912), CDA1 (SEQ ID NO:36 and SEQ ID NO:37), and CDA2 (SEQ ID NO:38 and SEQ ID NO:39). Different expression constructs will be transformed singly, or in combination, into strains of S. cerevisiae, and their effect on chitin and chitosan levels will be determined.

Different expression constructs can be developed in E. coli/yeast shuttle vectors such as yEp352ADH1, pPGK and pYES2 (see below). The constructs can be transformed into and maintained in yeast cells as free-replicating plasmids using appropriate selection. The expression constructs can also be subcloned into vectors suitable for gene integration into the yeast genome. One of such integration vector is YIp352 (Hill et al. 1986).

Several different strains of S. cerevisiae can be tested as hosts for glmS*54 expression and for chitin and chitosan production. For example, YNN281 (ATCC 204661) is a haploid strain that carries the ura3 mutation. Strains that carry mutations affecting cell wall synthesis and assembly, such as mmn9, fks1, and gas1, were shown to have a higher level of chitin and chitosan (Lagorce et al., 2002). It is anticipated that expression of glmS*54 will increase the chitin and chitosan content to even higher levels. The host strain can also be a diploid so that it is homozygous for the ura3 mutation, such as strain YPH501 (ATCC 204681). In addition, the diploid strains may also be developed to carry mutations in genes that affect cell wall, such as mmn9, fks1, and gas1.

For metabolic engineering to increase chitin and chitosan content, suitable genera of yeast include, but are not limited to, Saccharomyces, Schizosaccharomyces, Candida, Hansenula, Pichia, Kluyveromyces, and Phaffia. Suitable yeast species include, but are not limited to, Saccharomyces cerevisiae, Schizosaccharomyces pombe, Candida albicans, Candida Guillermondii, Hansenula polymorpha, Pichia pastoris, P. canadensis, Kluyveromyces marxianus and Phaffia rhodozyma.

Yeast cells can be transformed with recombinant plasmids using a variety of transformation methods. For example, the LiOAc method described by Geitz et al. (1995) can be used to transform S. cerevisiae with either high-copy number free-replicating plasmids, or with integration plasmids. Transformants will be isolated by selecting on medium lacking the appropriate nutrient. For example, transformants carrying the yEp352 and YIp352 expression vectors will be selected on SC-uracil medium (Adams et al., 1997).

Integration of gene expression cassettes in the yeast genome can be accomplished by using the methods described by Rothstein (1991). Integration of the Ylp352-based plasmid can be directed to the ADH1 locus by digesting the plasmid with BsrG I prior to transforming into yeast. This will cut the plasmid once, within the ADH1 promoter, and direct its integration to the ADH1 chromosomal locus through homologous recombination. Furthermore, strains carrying the plasmid integrated at ADH1 can be grown in a non-selective medium such as YPD (Adams et al., 1997) for several generations, and then plated onto medium containing 5-fluoroorotic acid (Adams et al., 1997). Colonies that arise on this medium are enriched for those cells that have undergone another recombination event to remove the plasmid and its associated URA3 gene. In some of these, the expression cassette will remain at the ADH1 locus. Although this process can be carried out with haploid yeast strains, it is preferred to do this with diploid strains so that one ADH1 allele is left intact. By isolating strains that have lost the integrated plasmid and associated URA3 gene, one can take the strain through additional rounds of plasmid integration, reusing the URA3 marker to select for the transformants.

Cloning of the E. coli glmS*54 in the Shuttle Vector yEp352ADH1

The E. coli mutant glmS*54 gene was cloned into expression vector yEp352ADH1. This vector is derived from yEp352 (Hill et al., 1986) by introducing the promoter and terminator sequences of the alcohol dehydrogenase I (ADH1). In S. cerevisiae, the ADH1 gene was expressed constitutively at high levels. The plasmid yEp352ADH1 replicates to multiple copies per yeast cell and has an ampicillin resistance marker for selection in E. coli and a URA3 marker for selection in yeast.

Primers nMD7107-021 and nMD7107-022 were designed for PCR amplification of the E. coli mutant glmS*54 gene (from ATG start codon to the stop codon). Forward primer nMD7107-021(Sac I) had the following sequences: 5′-AGC TGA GCT C ATG TGT GGA ATT GTT GGC GCG A-3′ (SEQ ID NO:1). Reverse primer nMD7107-022(Hind III) had the following sequence: 5′-TAC GAA GC TTA CTC AAC CGT AAC CGA TTT TGC-3′ (SEQ ID NO:2). Sequences specific to glmS*54 are highlighted by boldface letters.

Plasmid pKLN23-54 containing the E. coli glmS*54 gene (described in WO 04/003175A2) was used as the DNA templates in PCR reactions. A single band of PCR products of the expected size was generated under standard PCR conditions using the Taq polymerase. The PCR products were digested with restriction enzymes Sac I and Hind III, purified through agarose-gel and cloned into the yEp352ADH-1 vector that was predigested with the same enzymes. DNA ligation products were transformed into E. coli Top10 cells (Novagen) on ampicillin selection. First, 10 pools of colonies (10 per pool) were screened by PCR using the forward and reverse primers. Then, individual clones in positive pools were identified by PCR and confirmed by restriction digestions. Recombinant plasmids MD7107-238 and MD7107-239 contained the expression cassette of ADH1p-glmS*54-ADH1t.

Cloning of the E. coli glmS*54 in the Shuttle Vector pPGKptmcs

Plasmid pPGK is an E. coli/yeast shuttle vector containing the strong promoter of the phosphoglyceratekinase gene (PGK) and its terminator. The vector carries Amp^{R} and URA3 markers for selection in E. coli and yeast, respectively (Kang et al., 1990). Multiple cloning sites were introduced between the promoter and terminator to facilitate cloning of the gene to be expressed. The resulting vector was named pPGKptmcs. The added sites included unique sites of Bam HI, Mlu I, Xba I, Eag I, Not I, Hind III and Eco RI.

Plasmid pSWO7-8#4 (described in PCT Publication No. WO 040/03175A2) is a plasmid based on pCR-Script Amp SK(+) and contains the glmS*54 sequence (from ATG start codon to 177 bp down stream of the stop codon) cloned as a PCR product at the Srf I site. Previously, the glmS*54 sequence in this plasmid was functionally expressed in E. coli after subcloning into a pET vector. The g/mS*54 sequence was isolated by Not I and Bam HI digestions (sites provided by the vector). The 2.0-kb fragment was ligated to the Not I and Bam HI digested vector pPGKptmcs. Recombinant plasmids pBWO7-2#11 and pBWO7-2#19 contained the expression cassette of PGKp-glmS*54-PGKt. A short G+C rich nucleotide sequence was brought in between the promoter and the glmS coding sequence by subcloning (5′-ggc ggc cgc tct agc cag gtc tcc c, SEQ ID NO:3, 25 bp, G+C=80%). Part was in the original PCR primer (underlined) and the rest was from the pCR-Script.

Cloning of the E. coli glmS*54 in the Shuttle Vector pYES2

The shuttle vector pYES2 (Invitrogen) is a vector suitable for inducible expression of recombinant proteins in yeast. It contains the GAL1 promoter for induced expression by galactose. It also contains the CYC1 transcriptional terminator, a URA3 marker for selection in yeast and an ampicillin resistance marker for selection in E. coli.

The E. coli mutant glmS*54 sequence (from ATG start codon to 177 bp down stream of the stop codon) was excised and gel-purified from pSWO7-8#4 following restriction digestions with Sac I and Xho I. The 2.0-kb fragment was ligated to the Sac I and Xho I digested pYES2 vector. Recombinant plasmids pBWO7-3#2 and pBWO7-3#9 contained the expression cassette of GAL1p-glmS*54-CYC1t. A short G+C rich nucleotide sequence was brought in between the promoter and the glmS coding sequence by subcloning (5′-gga gct cca ccg cgg t ggc ggc cgc tct agc cag gtc tcc c, (SEQ ID NO:4), 41 bp, G+C=78%). Part was in the original PCR primer (underlined) and the rest was from the pCR-Script.

Cloning of the B. subtilis glmS Gene in the Vector pYES2.

pSWO7-15#8 contained the B. subtilis glmS coding sequence (represented herein by SEQ ID NO:26 and encoding the amino acid sequence of SEQ ID NO:27) cloned from strain 23856 as a PCR product at the Sfr I site in the pCR-Script Amp SK(+). As disclosed in WO04003175A2, the B. subtilis glmS gene was functionally expressed in E. coli, and the glucosamine synthase encoded by B. subtilis glmS was resistant to inhibition by glucosamine-6-phosphate. The glmS coding sequence (ATG through stop codon) was amplified by PCR from plasmid pSWO7-15#8 using forward primer 7107Bglm-5(5′ ATGC GGATTC ATG TGT GGA ATC GTA GGT TAT ATC CCT C 3′; SEQ ID NO:5) and reverse primer 07-13(5′ GATC CTCGAG TTA CTC CAC AGT AAC ACT CTT CGC AAGG 3′; SEQ ID NO:6). Primer 7107Bglm-5 had a BamHI site added (shown underlined) and primer 07-13 had an XhoI site added (shown underlined), for cloning into the pYES2 vector. The 1.8-kb PCR product was digested with Bam HI and Xho I and ligated to the pYES2 vector predigested with the same enzymes. The recombinant plasmids pBWO7-4#5 and pBWO7-4#10 contained the expression cassette of GAL1p-BsglmS-CYC1t.

Cloning of the S. cerevisiae GFA1 Gene in yEp352ADH1

S. cerevisiae GFA1 coding sequence (represented herein by SEQ ID NO:28 which encodes the amino acid sequence of SEQ ID NO:29) was PCR amplified from S288C genomic DNA with forward primer, 7107gfa-5(5′ AGGC GAATTC ATG TGT GGT ATC TTT GGT TAC TGC 3′; SEQ ID NO:7) and reverse primer 7107gfa-3(5′ AGGC CTGCAG TTA TTC GAC GGT AAC AGA TTT AGC 3′; SEQ ID NO:8). For cloning purposes, 7107gfa-5 had an Eco RI site and 7107gfa-3 had a Pst I site (shown underlined). The expected PCR product of 2154 bp was gel purified and digested with Eco RI and Pst I. The fragment was ligated to the Eco RI and Pst I digested yEp352ADH1 vector. Recombinant plasmid pBWO7-4#5 and pBWO7-4#10 contained the expression cassette of ADH1p-ScGFA1-ADH1t.

S. cerevisiae SWY5-deltaH cells were transformed with empty vectors yEp352ADH1, pPGKptmcs, pYES2, and expression plasmids constructed using this vectors. The LiOAc method described by Geitz et al. (1995) was used. The yeast strain has the ura and his auxotrophic selection markers. Yeast transformants were selected on plates of SCE-minus medium (Adams et al., 1997) supplemented with L-histidine at 20 mg 1⁻¹. Transformed yeast cell lines were grown in the same medium in shake flasks. Samples were taken at 24 or 48 hours to assay glucosamine and N-acetyglucosamine in the supernatant by colorimetric and HPLC methods. Cell samples were also analyzed for activities of glucosamine synthase (Table 1). Contents of chitin and chitosan in yeast biomass will be extracted and measured using the standard methods (Dallies et al., 1998). Chitin and chitosan in the biomass will be extracted by alkaline extraction. After acid hydrolysis or digestion with a commercial chitinase (Bulawa et al., 1986), glucosamine and N-acetyglucosamine produced will be assayed by colorimetric and HPLC method.

Demonstration of Glucosamine Synthase Overexpression in Yeast

Yeast cells harvested at fast growing phase or stationary phase showed only a very low level of glucosamine synthase activity. Yeast clones hosting free-replicating plasmids containing E. coli glmS*54, Bacillus glmS or S. cerevisiae GFA1 genes all had an increased glucosamine synthase activity (Table 1). As expected, the highest expression level was achieved using the yeast GFA1 gene. Overall, overexpression of glucosamine synthase resulted in an elevated content of N-acetylglucosamine. The changes in chitin and chitosan levels will be determined.

TABLE 1
Analysis of Yeast S. cerevisiae Transformants Generated with Different
Expression Constructs

| | | Enzyme Activity | | | |
|---|---|---|---|---|---|
| | | nmol/ | Fold | NAG/GlcN (mg/liter) | |
| Gene | Promoter | mg/min | Increase | Colorim. | HPLC (NAG) |
| | | GlmS | | | |
| | (Control)** | 1.1 | — | ND | ND |
| E. coli glmS*54 | ADH1 | 7.9 | 7 | 80-95 | 250 |
| | PGK-M*** | 3 | 3 | 15 | 600 |
| | GAL1-M*** | 3 | 3 | 45 | |
| B. subtilus glmS | GAl1 | 7.4 | 7 | 45 | |
| S. cerevisiae GFA1 | ADH1 | 200 | 200 | 100 | |
| | | GNA1 | | | |
| | (Control)** | 20 | — | ND | |
| S. cerevisiae GNA1 | PGK-M*** | 70 | 3 | ND | |
| | PGK | 5,400 | 270 | ND | |
| | GAL1-M*** | 70 | 3 | ND | |
| | GLA1 | 170 | 8 | ND | |

*A mutant variant of E. coli glmS gene. The mutant enzyme is resistant to product inhibition by glucosamine-6-phosphate.
**Yeast transformed with an empty vector yEp352-ADH1.
***A short G + C rich sequence was added between the promoter and the coding sequence.
ND: Not detectable (<5 mg/liter)

### Example 2

The following example describes the overexpression of S. cerevisiae GNA1 gene to increase chitin and chitosan levels in yeast.

Cloning of S. cerevisiae GNA1 in pADH313-956

Plasmid pADH313-956 is a low copy E. coli/yeast shuttle vector that contains ADH1 promoter and terminator. It also carried a HIS3 marker. The S. cerevisiae GNA1 coding sequence (represented herein by SEQ ID NO:32, which encodes the amino acid sequence SEQ ID NO:33) was previously cloned as a PCR product at the Srf I site in pCR-Script Amp SK(+), generating plasmid pSW07-60#3. As disclosed in WO 04/003175A2, the function of the sequence was demonstrated by high enzyme activity when the GNA1 protein was expressed in E. coli. The GNA1 sequence was isolated from pSWO7-60#3 by restriction digestions with EcoR I and Sac I. The fragment was then ligated into the EcoR I and Sac I sites of plasmid pADH313-956, generating the expression cassette of ADH1p-ScGNA1-ADH1t in plasmids pSWO7-114#1, #6, and #18. A short stretch of high G+C nucleotides was inserted immediately upstream of the ATG start codon (CCT GCA GCC CGG GGG ATC CGC CCG GAT CGG TCT CGC, SEQ ID NO:9, 36 bp, G+C=78%). This G+C rich nucleotide sequence was part of the PCR primer (underlined) or brought in from the pCR-Script vector.

S. cerevisiae SWY5-deltaH cells were transformed with empty vectors yADH313-956 and expression plasmids constructed using this vectors. The LiOAc method was used. The yeast strain has the ura and his auxotrophic selection markers. Yeast transformants were selected on plates of SCE-minus medium supplemented with uracile at 30 mg 1⁻¹. Transformed yeast cell lines were grown in the same medium in shake flasks. Samples were taken at 24 or 48 hours to assay glucosamine and N-acetyglucosamine in the supernatant by calorimetric and HPLC methods. Cell samples were also analyzed for activities of glucosamine synthase and glucosamine N-acetyltransferase (Table 1). The amount of chitin and chitosan in the biomass was also measured following alkaline extraction and acid hydrolysis.

Cloning of the S. cerevisiae GNA1 in pPGKptmcs

S. cerevisiae transformants generated with plasmids containing the cassette ADH1p-ScGNA1-ADH1t failed to show any increase in GNA1 enzyme activity. This was intriguing since it was a homologous gene and the function of the sequence had been confirmed by expression in E. coli. There appeared to be two possibilities: one was that the recombination of the ADH1 promoter and the GNA1 coding sequence could not form a functional unit for transcription and/or translation due to possible formation of unfavorable secondary structures. The second hypothesis was that the stretch of high G/C nucleotides inserted immediately upstream of the ATG start codon had a dramatic and negative impact on transcription and/or translation of the GNA1 gene.

In order to improve GNA1 expression in yeast, two different promoters were evaluated. One is the constitutive PGK promoter in the vector pPGKptmcs, and the other is the galactose-inducible GAL1 promoter in the vector pYES2. Moreover, two different expression cassettes were constructed using each promoter: one with and the other without the stretch of G+C rich nucleotide sequence to evaluate its effect on GNA1 expression.

To develop a GNA1 construct in pPGKptmcs with the G+C rich sequence, the EcoR 1-Not I fragment containing the GNA1 sequence with the high G/C stretch was isolated from pSWO7-60#3 and ligated at the EcoR I and Not I sites of pPGKptmcs. This resulted in the generation of pCALG60-1 and pCALG60-2.

To make a GNA1 construct without the G+C rich sequence, the GNA1 sequence was PCR amplified with a forward primer (containing an Eco RI site) and a reverse primer (containing a Sac I site) using pSWO7-60#3 plasmid DNA as template. The forward primers GN7107-001 had the following sequence: 5′-GAT CCG CCC GAT CGA ATT CAG C ATGAGC TTA C-3′; SEQ ID NO:10). The reverse primer GN7107-002 had the following sequence: 5′-GAT TAC GCC AAG CGC GCA ATT AAC CCT CAC TAA AG-3′; SEQ ID NO:11. The PCR product was digested with EcoR I and Not I, and ligated into pPGKptmcs, generating constructs without the stretch of high G+C sequence. These plasmids were named pCALG62-1 and pCALG62-2.

Cloning of the S. cerevisiae GNA1 in pYES2

Similar to the cloning of S. cerevisiae GNA1 in pPGKptmcs, two strategies were used to make GNA1 constructs in pYES2. The EcoR I—Not I fragment from pSWO7-60#3 was ligated into the EcoR I and Not I sites of pYES2. This resulted in the generation of pCALG61-1 and pCALG61-2.

PCR product with EcoR I and Not I ends were digested and ligated at the EcoR I and Not I sites of pYES2, generating plasmids pCALG63-1 and pCALG63-2.

Different expression constructs in pPGKptmcs or pYES2 were transformed into yeast as described above.

Demonstration of GNA1 Overexpression in Yeast

Wild-type yeast cells or cells transformed with an empty cloning vector showed only a very low level of glucosamine phosphate N-acetyltransferase (GNA1) activity. Yeast clones transformed with GNA1 expression plasmids showed a very variable level of transferase activity (Table 1). Clearly, the nature of the promoter had a dramatic effect on GNA1 expression. The highest expression level was observed using the PGK promoter. Under galactose induction, the GAL1 promoter also led to a significantly enhanced GNA1 expression. Interestingly, the presence of a short stretch of G+C rich nucleotides (25-41) placed immediately upstream of the ATG start codon reduced GNA1 expression substantially. This was observed with both pPGKptmcs and pYES2 vectors. The ADH1 promoter appeared to be very inefficient for driving GNA1 expression. Impact of GNA1 overexpression on content of N-acetylglucosamine appeared to be minor, suggesting that the reaction catalyzed by glucosamine synthase to be the rate-limiting step in the pathway. The effect of co-overexpression of GFA1 (especially glmS*54) and GNA1 on chitin and chitosan production will be determined (see Example below). Contents of chitin and chitosan in yeast biomass will be extracted and measured using the standard methods (Dallies et al., 1998). Chitin and chitosan in the biomass will be extracted by alkaline extraction. After acid hydrolysis or digestion with a commercial chitinase (Bulawa et al., 1986); glucosamine and N-acetyglucosamine produced will be assayed by colorimetric and HPLC method.

### Example 3

The following example describes overexpression of glucosamine-fructose amidotransferase genes (E. coli glmS*54 and S. cerevisiae GFA1) in fungi to increase the chitin and chitosan content.

Filamentous fungi, such as Aspergillus niger, are widely used in industrial production of enzymes (e.g. amylases, proteases, phytases and lipases) and chemicals (e.g. citric acid). Methods have been developed for genetic manipulation of filamentous fungi (see review by Berka and Barnett, 1989). Both antibiotic resistance (e.g. hygromycin, bleomycin, G418) or nutritional/auxotrophic marker (e.g. amdS, pyrG, argB, trpC) are available for the selection of transformants. The selection marker could be carried on the same vector as the gene construct to be transformed. Alternatively, the selection marker and the gene construct could be on separate linear or circular DNA fragments and be co-transformed into the fungal host. In general, transformants result from integration of DNA sequences into the host genome. Under certain circumstances, integration takes places via one or more recombination events at a site where the nucleotide sequence bears some homology to a portion of the vector DNA. More frequently, integration occurs at random chromosomal locations. Multiple copies of sequences could be integrated at the same site or at several different sites. Copy numbers of integrated sequences are variable among transformants. The number varies from one or two to as many as over 100 copies (Kelly and Hynes 1985). Usually, multiple copies of an introduced gene are optimal for high-level expression. However, there is no direct correlation between copy numbers and expression levels.

For metabolic engineering to increase chitin and chitosan content, suitable fungal hosts include, but are not limited to, Ascomycetes, Zygomycetes and Deuteromycetes. Suitable genus include, but are not limited to, Aspergillus, Absidia, Gongronella, Lentinus, Mucor, Phycomyces, Rhizopus, Chrysosporium, Neurospora and Trichoderma. Suitable fungal species include, but are not limited to, Aspergillus niger, Aspergillus terrreus, A. nidulans, Absidia coerulea; Absidia repens, Absidia blakesleeana, Gongronella butleri, Lentinus endodes, Mucor rouxii, Phycomyces blakesleenaus, Rhizopus oryzae, Chrysosporium lucknowense, Neurospora crassa, N. intermedia and Trichoderm reesei.

Glucoamylase encoded by the GLA gene was shown to be produced at very high levels in A. niger and other fungi. Molecular cloning of the GLA genomic DNA was reported by Boel et al. (1984). The GLA promoter and terminator sequences were used to direct high-level expression of various genes. To increase chitin and chitosan production, different expression cassettes were developed using the GLA promoter and terminator sequences.

The gpdA gene, encoding for glyceraldehyde-3-phosphate dehydrogenase (an important enzyme involved in glycolysis) is constitutively expressed at high levels in A. nidulans. Homologous and heterologous genes placed under the gpdA promoter control were expressed in A. niger at levels as high as 10-25% of total soluble protein. Pall and Brunelli (1993) constructed fungal expression vector pBARGPE1 containing the A. nidulans gpdA promoter and the A. nidulans trpC terminator. To increase chitin and chitosan production, different expression cassettes were developed using the pBARGPE1 vector.

Cloning of the GLA Sequence to Construct Expression Cassettes

The GLA genomic sequence was cloned from A. niger strain FGSC A733 by PCR amplification using genomic DNA as template. Genomic DNA was isolated using the YeaStar Genomic DNA kit (Zymo Research, Orange, Calif.). The protocol provided by the vendor was followed except that 0.5 mm zirconie/silica beads were added to enhance breakage of the cells during the vortexing steps. The cloned GLA sequence was 2602 bp and it covered the region from position −269, relative to the ATG start codon of the GLA coding sequence to position 161 downstream of the stop codon of the GLA sequence. The forward primer (GLA-NotI upper) contained a Not I site (underlined) and the GLA upstream sequence (boldface): 5′-gca tgc ggc cgc ttc gtc gcc taa tgt ctc g-3′; SEQ ID NO:12. The reverse primer (GLA-XhoI lower) contained an Xho I site (underlined) and the GLA downstream sequence (boldface): 5′-gca tct cga g ccc ggt gtc tgt att tcc gg-3′; SEQ ID NO:13. PCR was performed using the DNA polymerase Pfu ultra (Stratagene, La Jolla, Calif.). PCR product of expected size was digested with enzymes Not I and Sal I and cloned into pCR-Script Amp SK(+) predigested with the same enzymes. Recombinant plasmids were analyzed by restriction digestions. The GLA sequence in one of the plasmids (pGLA9) was partially sequenced from both ends. The sequence at the 3′ end (the C-terminal coding sequence and downstream sequence) matched exactly the sequence in GenBank (X00712.1, the entire sequence of which, including promoter and terminator, is represented herein by SEQ ID NO:40). However, nucleotide differences were found at two positions upstream of the ATG start codon: A (−239) and G (−31) were changed to G and A in pGAL9, respectively. These are probably strain variations.

The GLA sequence contained a unique restriction site of Bbv CI, located 1 bp upstream of the ATG start codon. The sequence also contained a unique restriction site of Sal I, situated 25 bp upstream of the stop codon. The Bbv CI-Sal I fragment in the GLA sequence could be deleted and replaced with the coding sequence of a different gene to be overexpressed in fungi.

Cloning of E. coli glmS*54 for Expression in Fungi

The E. coli gimS*54 coding sequence was amplified by PCR. The forward primer (BbvC upper) contained a Bbv CI site and the glmS*54 N-terminal coding sequence (boldface): 5′-GCA TCC TCA GC ATG TGT GGA ATT GTT GGC-3′; SEQ ID NO:14. The reverse primer (SalI lower) contained glmS*54 C-terminal coding sequence, the stop codon (bold face) and a Sal I restriction site: 5′-GCA TGT CGA C TTA CTC AAC CGT AAC CG-3′; SEQ ID NO:15. The PCR product of expected size was digested with Bbv CI and Sal I. The fragment was cloned into plasmid pGLA9 predigested with the same enzymes, generating the expression cassette of GLA1p-glmS*54-GLA1t in constructs pBW7113-2#1 and pBW7113-2#2.

The expression cassette was isolated as a Not I-Xho I fragment from pBW7113-2#1. As selection marker, the amdS sequence (5.2 kb) was isolated from plasmid p3SR2 by Eco RI and Sal I digestions. The amdS marker encodes for acetylamidase that allows transformants to use acetamide as the sole nitrogen source. The DNA fragments of GLA1p-glmS*54-GLA1t and amdS marker were co-transformed into protoplasts prepared from A. niger strain FGSC A733. The DNA fragment of expression construct and the amdS marker were co-transformed at a mass ratio of nine to one. A. niger protoplast preparation and transformation were carried out as described previously (Kelly and Hynes 1984).

The GLA1p-glmS*54-GLA1t cassette was also co-transformed into fungi with a hygromycin selection marker. Plasmid pBCX-hygro containing the hygromycin B gene (HmB) was obtained from FGSC. Circular and linearized plasmid DNA was co-transformed with fragments of expression cassette.

Transformed fungal cell lines will be grown in the appropriate media in shake flasks. Samples will be taken to assay glucosamine and N-acetyglucosamine in the supernatant by colorimetric and HPLC methods. Cell samples will also be analyzed for activities of enzyme activities. Contents of chitin and chitosan in fungal biomass will be extracted and measured using the standard methods (Dallies et al., 1998). Chitin and chitosan will be extracted by alkaline extraction. After acid hydrolysis or digestion with a commercial chitinase (Bulawa et al., 1986), glucosamine and N-acetyglucosamine produced will be assayed by colorimetric and HPLC method.

Cloning of S. cerevisiae GFA1 for Expression in Fungi

A yeast GFA1 coding sequence (represented herein by SEQ ID NO:28, encoding the amino acid sequence of SEQ ID NO:29) was PCR amplified from S. cerevisiae S288C genomic DNA with forward primer 7113-1 (5′-AGCT CCTCAGC A ATG TGT GGT ATC TTT GGT TAC TGC-3′; SEQ ID NO:16) and reverse primer 7113-2(5′ AGGC CTCGAG TTA TTC GAC GGT AAC AGA TTT AGC 3′; SEQ ID NO:17). Primer 7113-1 included a Bbv CI site and primer 7113-2 included an Xho I site (shown underlined). The 2154-bp PCR product was gel purified and digested with Bbv CI and Xho I. The fragment was ligated to the Bbv CI and Sal I digested pGLA9 vector, generating plasmids pBW7113-1#1 and pBW7113-1#20. The GLA1p-GFA1-CYC1t expression cassette was excised from pBW7113-1#1 with restriction endonucleases Not I and Xho I, and gel purified for transformation into A. niger as described above.

### Example 4

The following example describes overexpression of chitin deacetylase genes (S. cerevisiae CDA1 and CDA2) in fungi to increase chitosan content.

Chitin deacetylase (CDA) catalyzes the last step of chitosan synthesis pathway. CDA genes have been cloned from filamentous fungi Mucor rouxii and Gongronella butleri. Yeast S. cerevisiae has two CDA genes (CDA1 and CDA2) and both were cloned and functionally expressed in yeast. Since both CDA genes are free of introns (GenBank Accession number NC_{—}001144), PCR amplification was employed to clone the genes from yeast genomic DNA for overexpression in fungi. CDA1 and CDA2 sequences were cloned into pGLA9 to construct expression cassettes with the A. niger GLA promoter and terminator.

The CDA1 gene coding sequence (represented herein by SEQ ID NO:36, encoding the amino acid sequence of SEQ ID NO:37) was PCR amplified with primers GN7107-003 and GN7107-004 using as template genomic DNA isolated from S. cerevisiae strain ATCC28388. Primer GN7107-003 (5′-GCG GGG GCC TCA GCA ATG AAA ATT TTC AAT ACA ATA CAA TCT G-3′; SEQ ID NO:18) contained a Bbv CI site (underlined) and nucleotides identical to positions +1 to +25 (relative to the ATG start codon) of the CDA1 N-terminal coding sequence (bold face letters). Primer GN7107-004 (5′-GCG GGG GTC GAC CTA GTC GTA GCG TTC GAT G-3′; SEQ ID NO:19) contained nucleotides reverse complementary to the C-terminal coding sequence of CDA1 including the stop codon (bold face letters) and contained a Sal I recognition site (underlined). PCR using primers GN7107-003, GN7107-004 produced a product of the expected size for S. cerevisiae CDA1.

Similarly, CDA2 coding sequence (represented herein by SEQ ID NO:38, encoding the amino acid sequence of SEQ ID NO:39) was PCR amplified from S. cerevisiae strain ATCC28383 using forward primer GN7107-005 (5′-GCGGGGCCTCAGCA ATG AGA ATA CAA CTA AAT ACA ATT GAT TTG-3′; SEQ ID NO:20) and reverse primer CH7113-002 (5′-CTTCAATT-CCCGTCGAC TTA GGA CAA GAA TTC TTT TAT GTA ATC-3′; SEQ ID NO:21). The forward primer contained a Bbv CI and the reverse primer contained a Sal I site. PCR product of the expected size was generated.

PCR products of CDA1 and CDA2 were each digested with Bbv CI and Sal I and ligated with pGAL9 that was predigested with Bbv CI and Sal I. Recombinant plasmids containing the expression cassette GLAp-CDA1-CYC1t were named pCALC1 (sibling clones #1 and #2). CDA2 expression constructs were named pCALC2 (sibling clones #1 and #2).

CDA1 and CDA2 expression plasmids were digested with Not I and Xho I. DNA fragments of both expression cassettes were purified and transformed into A. niger as described above.

### Example 5

The following example describes overexpression of additional genes in fungi to increase the chitosan content.

Following the strategies and methods described above, additional chitin biosynthetic genes can be cloned into pGLA9-based vector, pBARGPE1-based vector or any other suitable vectors to increase chitin and chitosan production. The chitin biosynthetic genes from S. cerevisiae can be used, or, alternatively, genes from other sources including filamentous fungi can be identified and cloned. Currently, several chitin synthase genes from filamentous fungi, including A. nidulans, have been identified and cloned. Genes from S. cerevisiae, for example, that are known and that can be used for this purpose include, but are not limited to, GNA1 (SEQ ID NO:32 and SEQ ID NO:33), PCM1/AGM1 (nucleotide sequence X75816, amino acid sequence CAA53452.1), UAP1 (nucleotide sequence included in NC_{—}001136, amino acid sequence NP_{—}010180.1), CHS1 to CHS7 (P08004, P14180, P29465, NP 009492, NP_{—}013434, NP_{—}012436 and NP_{—}012011, respectively), other CHS genes such as Aspergillus niger CHS1_ASPNG (P30581), CHS2_ASPNG (P30582), A. fumigatus CHSC_ASPFU (Q92197), CHSD_ASPFU (P78746), CHSG_ASPFU (P54267), A. orzae chitin synthase (AAK31732.1), chsZ (BBB88127.1), and chsY (BAB88128.1), YEA4 (NP_{—}010912), CDA1 (SEQ ID NO:36 and SEQ ID NO:37), and CDA2 (SEQ ID NO:38 and SEQ ID NO:39). Genes from other yeast and other fungi can also be used as described herein (e.g., note the Candida albicans sequences described herein, among others). Different expression constructs can be transformed singly, or in combination, into fingi and their effects on chitin and chitosan levels will be determined.

Different vectors and selection markers could be used to express chitosan biosynthesis genes. For example, pyrG marker can be used in pyrG mutants (existing A. niger mutant strain ATCC 62590 or such mutant strains that can be isolated from the nature or following mutagenesis) as described by Goosen et al. (1987). The arg marker can be used for transformation of arg mutants (arginin auxotrophy).

### Example 6

The following example describes the selection of a suitable fungal strain as the metabolic engineering host to develop an industrial production strain for chitosan production.

The chitin and chitosan content and degree of deacetylation vary greatly according to filamentous fungi genus, species and strains (Rane and Hoover, 1993; Pochanavanich and Suntomsuk, 2002). For metabolic engineering to increase chitin and chitosan content, suitable fungal hosts include, but are not limited to, Ascomycetes, Zygomycetes and Deuteromycetes. Suitable genus include, but are not limited to, Aspergillus, Absidia, Gongronella, Lentinus, Mucor, Phycomyces, Rhizopus, Chrysosporium, Neurospora and Trichoderma. Suitable fungal species include, but are not limited to, Aspergillus niger, Aspergillus terrreus, A. nidulans, Absidia coerulea, Absidia repens, Absidia blakesleeana, Gongronella butleri, Lentinus endodes, Mucor rouxii, Phycomyces blakesleenaus, Rhizopus oryzae, Chrysosporium lucknowense, Neurospora crassa, N. intermedia and Trichoderm reesei.

Generally speaking, the vectors described above could be used to transform a variety of fungi species. Commonly used laboratory fungal strains were first used to demonstrate the impact of different vectors on chitosan production. However, to the goal of developing an industrial strain for chitosan production, strains from different genus and species will be screened to select the most suitable strain for metabolic engineering. The ideal host should-have the following characteristics: food grade or GRAS organism, high chitosan content, genetically transformable, fast growth and simple medium requirements.

Chitin and chitosan are common constituents of most fungal cell walls, including Euascomycetes, Zygomycetes, Chytridiomycetes, Ascomycetes, Basiodiomycetes and Sporobolomycetaceae, Mucor, Phycomyces, Absidia and other members of the order Mucorales. Chitin/chitosan content can vary greatly between different fungi. Published values for various filamentous fungi based on chitin as a percent of the cell wall range from a few percent to over 50 percent. Media and conditions of growth can affect chitin content. Chitin content has also been shown to vary significantly with growth phase. It is clear there exist large fluctuations in the composition of the cell wall from different fungi belonging to the same taxonomic group, and even of different strains of the same species. Some of this variability is due to the variety of different growth conditions and methods of chitin analysis used by different researchers.

The objective of the screening module is to identify several promising fungal strains having desirable characteristics for chitosan production. Desirable characteristics can be defined using the criteria listed below:

Chitin/chitosan content: The goal is to identify fungi having high chitosan content in the cell wall.

Growth characteristics: The organism should exhibit rapid growth, be easily cultured, capable of robust growth on inexpensive media, and achieve high biomass density while maintaining high chitosan content. The organism should be non-pathogenic and produce no harmful substances. GRAS or food grade organisms are preferable.

Chitin/chitosan recovery: After growth the biomass must be easily harvested and the chitin/chitosan readily extractable from the mycelia.

Examination of the literature suggests it would be prudent to examine a relatively large number of fungal strains (100-1000) across several different classes. Chitin and chitosan are found in several classes of fungi: Zygomycetes, Ascomycetes, Basidiomycetes, Deuteromycetes, Oomycetes and Hyphochytridiomycetes. Collections of fungi isolated from the environment for various screening programs could be used. Strains could also be obtained from commercial or other sources such as ATCC and Fungal Genetics Stock Center.

Fungi chosen for screening will include a broad range of genera representing all classes of fungi known to contain chitin or chitosan. For strain selection a certain bias will be employed towards genera representing industrially important fungi (Aspergillus and Rhizopus), and classes of fungi such as those belonging to the Zygomycetes (Absidia and Mucor) that contain chitosan as well as chitin.

Examining a large number of strains requires a flask screen. Spores or mycelia will be used to inoculate test tube cultures. These cultures will be used to inoculate flasks. Typically, fungi need several days to achieve good growth and maximize chitosan content. One basic growth medium will be employed in the screen, but it is anticipated that other growth media may be needed due to the variety of organisms examined. After significant growth has been obtained, mycelia will be harvested and the chitin/chitosan extracted using standard procedures. Alkali-insoluble cell wall material will be isolated and analyzed for chitin/chitosan content. This analysis will be used to determine chitin/chitosan content as a weight percentage of the dried mycelia biomass (mg chitin/g DCW) and as a weight percentage of alkali-insoluble material (cell walls) to allow direct comparison between fungal strains.

Strains having the best set of production characteristics will be confirmed by retesting. Based on these results, several good candidates will be further characterized as to growth rate, medium requirement and readiness for genetic manipulations. The most suitable strain will be selected for metabolic engineering and further optimization.

### Example 7

The following example describes random mutagenesis of genetically-engineered chitosan production strain and screening for further improved producers.

Classical mutagenesis is a way to introduce random mutations into the genome that may affect the strains ability to accumulate chitin/chitosan. Strains improved by targeted gene overexpression and gene deletion will be mutagenized and then selective techniques would be employed to select for the chitin/chitosan overproducers from the pool of mutants. Overproducing strains would then become parent strains for further rounds of mutagenesis.

Use of a classical strain improvement approach for improvement in chitin/chitosan yield represents a formidable technical challenge. The desired product is high molecular weight, does not diffuse out of the organism, and is not readily quantifiable in vivo. For strain improvement by random mutagenesis, cells or spores of the organism are treated with various mutagens (UV light, nitrosoguanidine, nitrous acid or ethyl methyl sulfonate) to generate random mutations throughout the genome. Such a family of mutants can be examined for improved productivity either by random plating and screening, or after exposing mutants to various conditions designed to identify or select for strains having improved productivity (rational strain improvement). Several primary screening approaches are outlined below.

Morphological screening. Frequently mutations dealing with cell wall biosynthesis give rise to mutants exhibiting visibly altered colonial morphology. After mutagenesis cells could be plated and colonies with altered morphology screened for chitin/chitosan content. Such altered morphologies could include lack of aerial mycelia, lack of spores, altered colony surface or texture, etc. Also of interest would be colonies having unusual size relative to the wild-type culture.

Fluorescence-activated cell sorting (FACS). Several dyes such as Calcofluor and Congo Red have been used in studies on chitin biosynthesis. These dyes selectively bind to chitin and can be used in fluorescence microscopy. Possibly FASC could be used to identify mutants having higher chitin content. Pilot experiments to determine feasibility of this method would have to be done first. If chitin content could be measured this way, mutagenized cells could be rapidly screened for enhanced chitin producers.

Feasibility studies involving the various screening methods will be tested with high and low chitin/chitosan producers. Once selective techniques are established a routine mutagenesis and selection will be performed. Candidates of improved clones will be further confirmed by shake flask experiments.

### Example 8

The following example describes methods for extracting and recovering (collecting) chitin and chitosan from yeast and fungal strains with elevated levels of chitin or chitosan, and are based on those described previously in U.S. Pat. No. 4,806,474, WO 0,168,714 and other publications (Rane and Hoover, 1993; Synowiecki and A1-Khateeb, 1997; Pochanavanich and Suntomsuk. 2002).

Following growth of the yeast and fungal strains, the microbial biomass is collected by filtration or centrifugation. The biomass is then treated with a hot alkaline solution, such as 35% sodium hydroxide, to remove contaminating materials such as proteins, minerals, etc. The remaining solids containing the chitin and/or chitosan are then collected, washed to remove the caustic solution, and resuspended in an acidic solution to solubilize the chitosan. The solution containing the chitosan is then recovered, and the solution is adjusted to pH 10 to cause precipitation of the chitosan. Precipitated chitosan is then collected by centrifugation. Contents of chitin and chitosan in biomass will be extracted and measured using the standard methods (Dallies et al., 1998). After acid hydrolysis or digestion with a commercial chitinase (Bulawa et. al., 1986), glucosamine and N-acetyglucosamine produced will be assayed by colorimetric and HPLC method.

Each reference cited or described herein is incorporated herein by reference in its entirety. In particular, each of: U.S. Pat. No. 6,372,457, PCT Publication No. WO 00/04182, PCT Publication No. WO 98/30713, and PCT Publication No. WO 04/003175A2 is incorporated herein by reference in its entirety. Similarly, each nucleic acid or amino acid sequence referenced by a database accession number cited or described herein is incorporated herein by reference in its entirety.

### REFERENCES

Adams, A., D. E. Gottschling, C. A. Kaiser, T. Stearns. 1997. Methods in Yeast Genetics, A Cold Spring Harbor Laboratory Course Manual. Cold Spring Harbor Laboratory Press, Plainview, N.Y.
Badet, B., P. Vermoote, and F. Le Goffic. 1988. Glucosamine synthetase from Escherichia coli: kinetic mechanism and inhibition by N³-fumaroyl-L-2,3-diaminopropionic derivatives. Biochemistry 27:2282-2287.
Berka, R. M. and C. C. Barnett. 1989. The development of gene expression systems for filamentous fungi. Biotech. Adv. 7:127-154.
Borgia, P. and C. L. Dodge. 1992. Characterization of Aspergillus nidulans mutants deficient in cell wall chitin or glucan. J. Bacteriol. 174:377-383.
Broschat K O., C. Gorka, J. D. Page, C. L. Martin-Berger, M. S. Davies, H. C. Huang, E. A. Gulve, W. J. Salsgiver and T. P. Kasten. 2002. Kinetic characterization of human glutamine-fructose-6-phosphate amidotransferase I: potent feedback inhibition by glucosamine 6-phosphate. J. Biol. Chem. 277:14764-14770.
Bulik D. A., M. Olczk, H. A. Lucero, R. C. Osmond, P. W. Robbins and C. A. Specht. 2003. Chitin synthesis in Saccharomyces cerevisiae in response to supplementation of growth medium with glucosamine and cell wall stress. Eukaryotic Cell, 2:886-900.
Bulawa, J. P., M. Slater, E. Cabib, J. Au-Young, A. Sburlati, W. L. Adair and P. Robbins. 1986. The S. cerevisiae structural gene for chitin synthase is not required for chitin synthesis in vivo. Cell 46:213-225.
Dallies, N., J. Francois and V. Paquer. 1998. A new methods for quantitative determination of polysaccharides in the yeast cell wall. Application to the cell wall defective mutants of Saccharomyces cerevisiae. Yeast 14:1297-1306.
Fan, W., J. A. Bohlmann, J. R. Trinkle, J. D. Steinke, K. Hwang and J. P. Henning. 2000. Chitosan and method of preparing chitosan. WO0168714A1.
Farkas, V. 1989. Polysaccharide metabolism. In “The Yeast, volume 3: Metabolism and Physiology of Yeasts,” Rose A. H. and Harrison J. S., eds., Academic Press, London.
Geitz, R. D., R. H. Schiestl, A. R. Willems, and R. A. Woods. 1995. Studies on the transformation of intact yeast cells by the LiAc/SS-DNA/PEG procedure. Yeast 11:355-360.
Goosen, T., G. Bloemheuvel, C. Gysler, D. A. de Bie, H. W. van den Broek and K. Swart. 1987. Transformation of Aspergillus niger using the homologous orotidine-5′-phosphate-decarboxylase gene. Curr. Genet. 11:499-503.
Hershberger, D. F. 1985. Preparation of mycelial chitosan and glucan fraction from microbial biomass. U.S. Pat. No. 4,806,474.
Hill, J. E., A. M. Myers, T. J. Koerner, and T. J. Tzagoloff. 1986. Yeast/E. coli shuttle vectors With multiple unique restriction sites. Yeast 2:163-167.
Kang, Y. S., J. Kane, J. Kurjan, J. M. Stadel and D. J. Tipper. 1990. Effects of expression of mammalian Ga and hybrid mammalian-yeast Ga proteins on the yeast pheromone response signal transduction pathway. Mol. Cell. Biol. 10:2582-2590.
Kelly J. M. and M. J. Hynes. 1985. Transformation of Aspergillus niger by the amdS gene of Aspergillus nidulans. The EMBO J. 4:475-479.
Knorr, D. 1991. Recovery and utilization of chitin and chitosan in food processing waste management. Food Technol. 26:114-122.
Lagorece, A., V. Le Berre-Anton, B. Aguilar-Uscanga, H. Martin-Yken, A. Dagkessamanskaia and J. Francois. Involvement of GFA1, which encodes glutamine-fructose-6-phosphate amidotransferase, in the activation of the chitin synthesis pathway in response to cell-wall defects in Saccharomyces cerevisiae. Eur. J. Biochem. 269:1697-1707.
Maw, T., T. K Tan, E. Khor and S. M. Wong. 2002. Selection of Gongronella butleri strains for enhanced chitosan yield with UV mutagenesis. J. Biotechnology. 95:189-193.
McGahren, W. J., G. A. Perkinson, J. A. Growich, R. A. Leese and G. A. Ellestad. 1984. Chitosan by fermentation. Process Biochem. 19:88-90.
McKnight, G. L., S. L. Mudri, S. L. Mathewes, R. R. Traxinger, S. Marshall, P. O. Sheppard and P. J. O'Hara. Molecular cloning, cDNA sequence, and bacterial expression of human glutamine:fructose-6-phosphate amidotransferase. J. Biol. Chem. 267:25208-25212.
Muller, C., M. McIntyre, K Hansen and J. Nielson. 2002. Metabolic engineering of the morphology of Aspergillus oryzae by altering chitin synthesis. Appl. Environ. Microbiol. 68:1827-1836.
Pall, M. L. and J. P. Brunelli. 1993. A series of six compact fungal transformation vectors containing polylinkers with multiple unique restriction sites. Fungal Genetic News Letter 40:59-62.
Pochanavanich, P. and W. Suntornsuk. 2002. Fungal chitosan production and its characterization. Lett; Appl. Microbiol. 35:17-21.
Rane, K. D. and D. G. Hoover. 1993. Production of chitosan by fingi. Food Biotechnol. 7:11-33.
Rothstein, R. 1991. Methods in Enzymology, Guide to Yeast Genetics and Molecular Biology. 194:281-301.
Roncero, C. 2002. The genetic complexity of chitin synthesis in fungi. Curr. Genet. 6:367-378.
Synowiecki, J. and N. A. A. Q. Al-Khateeb. 1997. Mycelia of Mucor rouxii as a source of chitin and chitosan. Food Chemistry 60:605-610.
Velichkov, A. D. and G. S. Georgiev. 1991. An evaluation of the chitin content in waste mycelium from four strains fungi of industrial importance. Acta Microbiol. Bulgarica 28:69-74.
White, R. J. 1968. Control of amino sugar metabolism in Escherichia coli and isolation of mutants unable to degrade amino sugars. Biochem. J. 106:847-858.

While various embodiments of the present invention have been described in detail, it is apparent that modifications and adaptations of those embodiments will occur to those skilled in the art. It is to be expressly understood, however, that such modifications and adaptations are within the scope of the present invention, as set forth in the following claims.

1. A method to produce chitin or chitosan by a fermentation process, comprising:
a) culturing in a fermentation medium a microorganism which comprises at least one genetic modification selected from the group consisting of:
i) a genetic modification that results in an increase in the activity of glutamine-fructose-6-phosphate amidotransferase;
ii) a genetic modification that results in an increase in the activity of glucosamine-6-P acetyltransferase;
iii) a genetic modification that results in an increase in the activity of chitin synthase;
iv) a genetic modification that results in an increase in the activity of chitin deacetylase;
v) a genetic, modification that results in a decrease in the activity of N-acetylglucosamine-6-P deacetylase;
vi) a genetic modification that results in a decrease in the activity of glucosamine-6-P deaminase;
vii) a genetic modification that results in a decrease in the activity of chitinase; and
viii) a genetic modification that results in a decrease in the activity of chitosanase; and
b) collecting a product produced from the step of culturing which is selected from the group consisting of chitin and chitosan.

2. The method of claim 1, wherein the glutamine-fructose-6-P amidotransferase is resistant to inhibition by UDP-N-acetylglucosamine.

3. The method of claim 1, wherein the glutamine-fructose-6-P amidotransferase is resistant to inhibition by glucosamine-6-phosphate.

4. The method of claim 1, wherein the glutamine-fructose-6-P amidotransferase is resistant to inhibition by glutamate.

5. The method of claim 1, wherein the microorganism has a genetic modification that increases the activity of glutamine-fructose-6-phosphate amidotransferase, and wherein the genetic modification comprises transforming the microorganism with a recombinant nucleic acid molecule encoding the glutamine-fructose-6-phosphate amidotransferase or a biologically active homologue thereof.

6. The method of claim 5, wherein the recombinant nucleic acid molecule comprises the coding region of yeast, fungal, plant or animal GFA1.

7. The method of claim 5, wherein the recombinant nucleic acid molecule comprises the coding region of bacterial GlmS.

8. The method of claim 5, wherein the glutamine-fructose-6-phosphate amidotransferase is resistant to inhibition by UDP-N-acetylglucosamine.

9. The method of claim 5, wherein the glutamine-fructose-6-phosphate amidotransferase is resistant to inhibition by glucosamine-6-phosphate.

10. The method of claim 5, wherein the glutamine-fructose-6-phosphate amidotransferase is resistant to inhibition by glutamate.

11. The method of claim 1, wherein the microorganism comprises a genetic modification that results in an increase in the activity of glucosamine-6-P acetyltransferase.

12. The method of claim 1, wherein the microorganism comprises a genetic modification that results in an increase in the activity of chitin synthase.

13. The method of claim 1, wherein the microorganism comprises a genetic modification that results in an increase in the activity of chitin deacetylase.

14. The method of claim 1, wherein the microorganism comprises a genetic modification that results in an increase in the activity of chitin synthase and a genetic modification that results in an increase in the activity of chitin deacetylase.

15. The method of claim 1, wherein the microorganism comprises a genetic modification that results in a decrease in the activity of glucosamine-6-P deaminase.

16. The method of claim 1, wherein the microorganism comprises a genetic modification that results in a decrease in the activity of N-acetylglucosamine-6-P deacetylase.

17. The method of claim 1, wherein the microorganism comprises a genetic modification that results in a decrease in the activity of N-acetylglucosamine-6-P deacetylase and a genetic modification that results in a decrease in the activity of glucosamine-6-P deaminase.

18. The method of claim 1, wherein the microorganism comprises a genetic modification that results in a decrease in the activity of chitinase.

19. The method of claim 1, wherein the microorganism comprises a genetic modification that results in a decrease in the activity of chitosanase.

20. The method of claim 1, wherein the microorganism comprises a genetic modification that results in a decrease in the activity of chitinase and a genetic modification that results in a decrease in the activity of chitosanase.

21. The method of claim 1, wherein the microorgansim is a fungus.

22. The method of claim 1, wherein the microorganism is a yeast.

23. The method of claim 1, wherein the microorganism is a yeast selected from the group consisting of Saccharomyces and Schizosaccharomyces.

24. The method of claim 1, wherein the microorganism is a filamentous fungus.

25. The method of claim 1, wherein the microorganism is a filamentous fungus selected from the group consisting of Aspergillus, Absidia and Rhizopus.

26. The method of claim 1, wherein the microorganism is selected from the group consisting of S. cerevisiae, A. niger, and A. nidulans.

27. The method of claim 1, wherein the genetic modifications increase the content of chitin or chitosan in the cell wall of the microorganism as compared to the wild-type microorganism by at least about 50%.

28. The method of claim 1, wherein the genetic modifications increase the content of chitin or chitosan in the cell wall of the microorganism as compared to the wild-type microorganism by at least about 2 fold.

29. The method of claim 1, wherein the genetic modifications increase the content of chitin or chitosan in the cell wall of the microorganism as compared to the wild-type microorganism by at least about 5 fold.

30. The method of claim 1, wherein the genetic modifications increase the content of chitin or chitosan in the cell wall of the microorganism as compared to the wild-type microorganism by at least about 10 fold.

31. The method of claim 1, wherein the step of collecting comprises treatment of microorganism cells with a hot alkaline solution, collection and washing of the remaining solids containing chitin or chitosan, resuspension of the washed solids in an acidic solution to solubilize the chitin or chitosan, and precipitation of the chitin or chitosan.

32. A microbial biomass comprising chitin and/or chitosan and produced by the method of claim 1.

33. A genetically modified microorganism comprising at least two genetic modifications selected from the group consisting of:
a) a genetic modification that results in an increase in the activity of glutamine-fructose-6-phosphate amidotransferase;
b) a genetic modification that results in an increase in the activity of glucosamine-6-p acetyltransferase;
c) a genetic modification that results in an increase in the activity of chitin synthase;
d) a genetic modification that results in an increase in the activity of chitin deacetylase;
e) a genetic modification that results in a decrease in the activity of N-acetylglucosamine-6-P deacetylase;
f) a genetic modification that results in a decrease in the activity of glucosamine-6-P deaminase;
g) a genetic modification that results in a decrease in the activity of chitinase; and
h) a genetic modification that results in a decrease in the activity of chitosanase.

34. The genetically modified microorganism of claim 33, wherein the genetically modified microorganism is a filamentous fungus.

35. The genetically modified microorganism of claim 33, wherein the genetically modified microorganism is a yeast.


SEQUENCE LISTING (59 sequences)

SEQ ID NO: 1 - DNA, 32 bp, Artificial sequence
  primer
agctgagctcatgtgtggaattgttggcgcga

SEQ ID NO: 2 - DNA, 32 bp, Artificial sequence
  primer
tacgaagcttactcaaccgtaaccgattttgc

SEQ ID NO: 3 - DNA, 25 bp, Artificial sequence
  primer
ggcggccgctctagccaggtctccc

SEQ ID NO: 4 - DNA, 41 bp, Artificial sequence
  primer
ggagctccaccgcggtggcggccgctctagccaggtctccc

SEQ ID NO: 5 - DNA, 38 bp, Artificial sequence
  primer
atgcggattcatgtgtggaatcgtaggttatatccctc

SEQ ID NO: 6 - DNA, 38 bp, Artificial sequence
  primer
gatcctcgagttactccacagtaacactcttcgcaagg

SEQ ID NO: 7 - DNA, 34 bp, Artificial sequence
  primer
aggcgaattcatgtgtggtatctttggttactgc

SEQ ID NO: 8 - DNA, 34 bp, Artificial sequence
  primer
aggcctgcagttattcgacggtaacagatttagc

SEQ ID NO: 9 - DNA, 36 bp, Artificial sequence
  primer
cctgcagcccgggggatccgcccggatcggtctcgc

SEQ ID NO: 10 - DNA, 32 bp, Artificial sequence
  primer
gatccgcccgatcgaattcagcatgagcttac

SEQ ID NO: 11 - DNA, 35 bp, Artificial sequence
  primer
gattacgccaagcgcgcaattaaccctcactaaag

SEQ ID NO: 12 - DNA, 31 bp, Artificial sequence
  primer
gcatgcggccgcttcgtcgcctaatgtctcg

SEQ ID NO: 13 - DNA, 30 bp, Artificial sequence
  primer
gcatctcgagcccggtgtctgtatttccgg

SEQ ID NO: 14 - DNA, 29 bp, Artificial sequence
  primer
gcatcctcagcatgtgtggaattgttggc

SEQ ID NO: 15 - DNA, 27 bp, Artificial sequence
  primer
gcatgtcgacttactcaaccgtaaccg

SEQ ID NO: 16 - DNA, 36 bp, Artificial sequence
  primer
agctcctcagcaatgtgtggtatctttggttactgc

SEQ ID NO: 17 - DNA, 34 bp, Artificial sequence
  primer
aggcctcgagttattcgacggtaacagatttagc

SEQ ID NO: 18 - DNA, 43 bp, Artificial sequence
  primer
gcgggggcctcagcaatgaaaattttcaatacaatacaatctg

SEQ ID NO: 19 - DNA, 31 bp, Artificial sequence
  primer
gcgggggtcgacctagtcgtagcgttcgatg

SEQ ID NO: 20 - DNA, 44 bp, Artificial sequence
  primer
gcggggcctcagcaatgagaatacaactaaatacaattgatttg

SEQ ID NO: 21 - DNA, 44 bp, Artificial sequence
  primer
cttcaattcccgtcgacttaggacaagaattcttttatgtaatc

SEQ ID NO: 22 - DNA, 1830 bp, Escherichia coli
  CDS (1)..(1830)
atgtgtggaattgttggcgcgatcgcgcaacgtgatgtagcagaaatccttcttgaaggtttacgtcgtctggaataccgcggatatgactctgccggtctggccgttgttgatgcagaaggtcatatgacccgcctgcgtcgcctcggtaaagtccagatgctggcacaggcagcggaagaacatcctctgcatggcggcactggtattgctcacactcgctgggcgacccacggtgaaccttcagaagtgaatgcgcatccgcatgtttctgaacacattgtggtggtgcataacggcatcatcgaaaaccatgaaccgctgcgtgaagagctaaaagcgcgtggctataccttcgtttctgaaaccgacaccgaagtgattgcccatctggtgaactgggagctgaaacaaggcgggactctgcgtgaggccgttctgcgtgctatcccgcagctgcgtggtgcgtacggtacagtgatcatggactcccgtcacccggataccctgctggcggcacgttctggtagtccgctggtgattggcctggggatgggcgaaaactttatcgcttctgaccagctggcgctgttgccggtgacccgtcgctttatcttccttgaagagggcgatattgcggaaatcactcgccgttcggtaaacatcttcgataaaactggcgcggaagtaaaacgtcaggatatcgaatccaatctgcaatatgacgcgggcgataaaggcatttaccgtcactacatgcagaaagagatctacgaacagccgaacgcgatcaaaaacacccttaccggacgcatcagccacggtcaggttgatttaagcgagctgggaccgaacgccgacgaactgctgtcgaaggttgagcatattcagatcctcgcctgtggtacttcttataactccggtatggtttcccgctactggtttgaatcgctagcaggtattccgtgcgacgtcgaaatcgcctctgaattccgctatcgcaaatctgccgtgcgtcgtaacagcctgatgatcaccttgtcacagtctggcgaaaccgcggataccctggctggcctgcgtctgtcgaaagagctgggttaccttggttcactggcaatctgtaacgttccgggttcttctctggtgcgcgaatccgatctggcgctaatgaccaacgcgggtacagaaatcggcgtggcatccactaaagcattcaccactcagttaactgtgctgttgatgctggtggcgaagctgtctcgcctgaaaggtctggatgcctccattgaacatgacatcgtgcatggtctgcaggcgctgccgagccgtattgagcagatgctgtctcaggacaaacgcattgaagcgctggcagaagatttctctgacaaacatcacgcgctgttcctgggccgtggcgatcagtacccaatcgcgctggaaggcgcattgaagttgaaagagatctcttacattcacgctgaagcctacgctgctggcgaactgaaacacggtccgctggcgctaattgatgccgatatgccggttattgttgttgcaccgaacaacgaattgctggaaaaactgaaatccaacattgaagaagttcgcgcgcgtggcggtcagttgtatgtcttcgccgatcaggatgcgggttttgtaagtagcgataacatgcacatcatcgagatgccgcatgtggaagaggtgattgcaccgatcttctacaccgttccgctgcagctgctggcttaccatgtcgcgctgatcaaaggcaccgacgttgaccagccgcgtaacctggcaaaatcggttacggttgagtaa

SEQ ID NO: 23 - protein, 609 aa, Escherichia coli
MCGIVGAIAQRDVAEILLEGLRRLEYRGYDSAGLAVVDAEGHMTRLRRLGKVQMLAQAAEEHPLHGGTGIAHTRWATHGEPSEVNAHPHVSEHIVVVHNGIIENHEPLREELKARGYTFVSETDTEVIAHLVNWELKQGGTLREAVLRAIPQLRGAYGTVIMDSRHPDTLLAARSGSPLVIGLGMGENFIASDQLALLPVTRRFIFLEEGDIAEITRRSVNIFDKTGAEVKRQDIESNLQYDAGDKGIYRHYMQKEIYEQPNAIKNTLTGRISHGQVDLSELGPNADELLSKVEHIQILACGTSYNSGMVSRYWFESLAGIPCDVEIASEFRYRKSAVRRNSLMITLSQSGETADTLAGLRLSKELGYLGSLAICNVPGSSLVRESDLALMTNAGTEIGVASTKAFTTQLTVLLMLVAKLSRLKGLDASIEHDIVHGLQALPSRIEQMLSQDKRIEALAEDFSDKHHALFLGRGDQYPIALEGALKLKEISYIHAEAYAAGELKHGPLALIDADMPVIVVAPNNELLEKLKSNIEEVRARGGQLYVFADQDAGFVSSDNMHIIEMPHVEEVIAPIFYTVPLQLLAYHVALIKGTDVDQPRNLAKSVTVE

SEQ ID NO: 24 - DNA, 1830 bp, Escherichia coli
  CDS (1)..(1830)
atgtgtggaattgttggcgcgatcgcgcaacgtgatgtagcagaaatccttcttgaaggtttacgtcgtctggaataccgcggatatgactctgccggtctggccgttgttgatacagaaggtcatatgacccgcctgcgtcgcctcggtaaagtccagatgctggcacaggcagcggaagaacatcctctgcatggcggcactggtattgctcacactcgctgggcgacccacggtgaaccttcagaagtgaatgcgcatccgcatgtttctgaacacattgtggtggtgcataacggcatcatcgaaaaccatgaaccgctgcgtgaagagctaaaagcgcgtggctataccttcgtttctgaaaccgacaccgaagtgattgcccatctggtgaactgggagctgaaacaaggcgggactctgcgtgaggccgttctgcgtgctatcccgcagctgcgtggtgcgtacggtacagtgatcatggactcccgtcacccggataccctgctggcggcacgttctggtagtccgctggtgattggcctggggatgggcgaaaactttatcgcttctgaccagctggcgctgttgccggtgacccgtcgctttatcttccttgaagagggcgatattgcggaaatcactcgccgttcggtaaacatcttcgataaaactggcgcggaagtaaaacgtcaggatatcgaatccaatctgcaatatgacgcgggcgataaaggcatttactgtcactacatgcagaaagagatctacgaacagccgaacgcgatcaaaaacacccttaccggacgcatcagccacggtcaggttgatttaagcgagctgggaccgaacgccgacgaactgctgtcgaaggttgagcatattcagatcctcgcctgtggtacttcttataactccggtatggtttcccgctactggtttgaatcgctagcaggtattccgtgcgacgtcgaaatcgcctccgaattccgctatcgcaaatctgccgtgcgtcgtaacagcctgatgatcaccttgtcacagtctggcgaaaccgcggataccctggctggcctgcgtctgtcgaaagagctgggttaccttggttcactggcaatctgtaacgttccgggttcttctctggtgcgcgaatccgatctggcgctaatgaccaacgcgggtacagaaatcggcgtggcatccactaaagcattcaccactcagttaactgtgctgttgatgctggtggcgaagctgtctcgcctgaaaggtctggatgcctccattgaacatgacatcgtgcatggtctgcaggcgctgccgagccgtattgagcagatgctgtctcaggacaaacgcattgaagcgctggcagaagatttctctgacaaacatcacgcgctgttcctgagccgtggcgatcagtacccaatcgcgctggaaggcgcattgaagttgaaagagatctcttacattcacgctgaagcctacgctgctggcgaactgaaacacggtccgctggcgctaattgatgccgatatgccggttattgttgttgcaccgaacaacgaattgctggaaaaactgaaatccaacattgaagaagttcgcgcgcgtggcggtcagttgtatgtcttcgccgatcaggatgcgggttttgtaagtagcgataacatgcacatcatcgagatgccgcatgtggaagaggtgattgcaccgatcttctacaccgttccgctgcagctgctggcttaccatgtcgcgctgatcaaaggcaccgacgttgaccagccgcgtaacctggcaaaatcggttacggttgagtaa

SEQ ID NO: 25 - protein, 609 aa, Escherichia coli
MCGIVGAIAQRDVAEILLEGLRRLEYRGYDSAGLAVVDTEGHMTRLRRLGKVQMLAQAAEEHPLHGGTGIAHTRWATHGEPSEVNAHPHVSEHIVVVHNGIIENHEPLREELKARGYTFVSETDTEVIAHLVNWELKQGGTLREAVLRAIPQLRGAYGTVIMDSRHPDTLLAARSGSPLVIGLGMGENFIASDQLALLPVTRRFIFLEEGDIAEITRRSVNIFDKTGAEVKRQDIESNLQYDAGDKGIYCHYMQKEIYEQPNAIKNTLTGRISHGQVDLSELGPNADELLSKVEHIQILACGTSYNSGMVSRYWFESLAGIPCDVEIASEFRYRKSAVRRNSLMITLSQSGETADTLAGLRLSKELGYLGSLAICNVPGSSLVRESDLALMTNAGTEIGVASTKAFTTQLTVLLMLVAKLSRLKGLDASIEHDIVHGLQALPSRIEQMLSQDKRIEALAEDFSDKHHALFLSRGDQYPIALEGALKLKEISYIHAEAYAAGELKHGPLALIDADMPVIVVAPNNELLEKLKSNIEEVRARGGQLYVFADQDAGFVSSDNMHIIEMPHVEEVIAPIFYTVPLQLLAYHVALIKGTDVDQPRNLAKSVTVE

SEQ ID NO: 26 - DNA, 1803 bp, Bacillus subtilis
  CDS (1)..(1803)
atgtgtggaatcgtaggttatatcggtcagcttgatgcgaaggaaattttattaaaagggttagagaagcttgagtatcgcggttatgactctgctggtattgctgttgccaacgaacagggaatccatgtgttcaaagaaaaaggacgcattgcagatcttcgtgaagttgtggatgccaatgtagaagcgaaagccggaattgggcatactcgctgggcgacacacggcgaaccaagctatctgaacgctcacccgcatcaaagcgcactgggccgctttacacttgttcacaacggcgtgatcgagaactatgttcagctgaagcaagagtatttgcaagatgtagagctcaaaagtgacaccgatacagaagtagtcgttcaagtaatcgagcaattcgtcaatggaggacttgagacagaagaagcgttccgcaaaacacttacactgttaaaaggctcttatgcaattgctttattcgataacgacaacagagaaacgatttttgtagcgaaaaacaaaagccctctattagtaggtcttggagatacattcaacgtcgtagcatctgatgcgatggcgatgcttcaagtaaccaacgaatacgtagagctgatggataaagaaatggttatcgtcactgatgaccaagttgtcatcaaaaaccttgatggtgacgtgattacacgtgcgtcttatattgctgagcttgatgccagtgatatcgaaaaaggcacgtaccctcactacatgttgaaagaaacggatgagcagcctgttgttatgcgcaaaatcatccaaacgtatcaagatgaaaacggcaagctgtctgtgcctggcgatatcgctgccgctgtagcggaagcggaccgcatctatatcattggctgcggaacaagctaccatgcaggacttgtcggtaaacaatatattgaaatgtgggcaaacgtgccggttgaagtgcatgtagcgagtgaattctcctacaacatgccgcttctgtctaagaaaccgctcttcattttcctttctcaaagcggagaaacagcagacagccgcgcggtactcgttcaagtcaaagcgctcggacacaaagccctgacaatcacaaacgtacctggatcaacgctttctcgtgaagctgactatacattgctgcttcatgcaggccctgagatcgctgttgcgtcaacgaaagcatacactgcacaaatcgcagttctggcggttcttgcttctgtggctgctgacaaaaatggcatcaatatcggatttgacctcgtcaaagaactcggtatcgctgcaaacgcaatggaagctctatgcgaccagaaagacgaaatggaaatgatcgctcgtgaatacctgactgtatccagaaatgctttcttcatcggacgcggccttgactacttcgtatgtgtcgaaggcgcactgaagctgaaagagatttcttacatccaggcagaaggttttgccggcggtgagctaaagcacggaacgattgccttgatcgaacaaggaacaccagtattcgcactggcaactcaagagcatgtaaacctaagcatccgcggaaacgtcaaagaagttgctgctcgcggagcaaacacatgcatcatctcactgaaaggcctagacgatgcggatgacagattcgtattgccggaagtaaacccagcgcttgctccgttggtatctgttgttccattgcagctgatcgcttactatgctgcactgcatcgcggctgtgatgtggataaacctcgtaaccttgcgaagagtgttactgtggagtaa

SEQ ID NO: 27 - protein, 600 aa, Bacillus subtilis
MCGIVGYIGQLDAKEILLKGLEKLEYRGYDSAGIAVANEQGIHVFKEKGRIADLREVVDANVEAKAGIGHTRWATHGEPSYLNAHPHQSALGRFTLVHNGVIENYVQLKQEYLQDVELKSDTDTEVVVQVIEQFVNGGLETEEAFRKTLTLLKGSYAIALFDNDNRETIFVAKNKSPLLVGLGDTFNVVASDAMAMLQVTNEYVELMDKEMVIVTDDQVVIKNLDGDVITRASYIAELDASDIEKGTYPHYMLKETDEQPVVMRKIIQTYQDENGKLSVPGDIAAAVAEADRIYIIGCGTSYHAGLVGKQYIEMWANVPVEVHVASEFSYNMPLLSKKPLFIFLSQSGETADSRAVLVQVKALGHKALTITNVPGSTLSREADYTLLLHAGPEIAVASTKAYTAQIAVLAVLASVAADKNGINIGFDLVKELGIAANAMEALCDQKDEMEMIAREYLTVSRNAFFIGRGLDYFVCVEGALKLKEISYIQAEGFAGGELKHGTIALIEQGTPVFALATQEHVNLSIRGNVKEVAARGANTCIISLKGLDDADDRFVLPEVNPALAPLVSVVPLQLIAYYAALHRGCDVDKPRNLAKSVTVE

SEQ ID NO: 28 - DNA, 2154 bp, Saccharomyces cerevisiae
  CDS (1)..(2154)
atgtgtggtatctttggttactgcaattatctagtggaaagatccagaggagaaattatcgacaccttagtggatggtttacaaagattagaatatagaggctatgattccaccggtattgctatcgatggtgacgaagctgattctactttcatctataagcaaatcggtaaagtgagtgctttgaaagaggagattactaagcaaaatccgaacagagacgttacttttgtctctcattgtggtattgcgcatactagatgggctactcacggtcgaccagaacaagttaactgtcaccctcaaagatctgacccagaagaccaatttgtggtcgttcataatggtatcatcacaaattttagagaactgaagactcttttaattaacaaaggttataaattcgaaagtgataccgataccgagtgtattgctaaactatatttgcatttatacaatacaaatttacaaaatgggcatgacttagatttccacgaattaaccaagctagttcttttagaactagaaggttcatacgggttattatgtaaatcttgtcactatcctaatgaggttatcgccactagaaaagggtcccctttactgattggtgtcaaatctgaaaaaaaactaaaagtcgacttcgtggatgtggaatttcccgaagaaaacgctggtcaaccggaaattccattgaaatctaacaacaaatcatttggcttgggcccaaagaaagctcgtgaatttgaagctggttcccaaaatgccaatttactaccaattgccgccaatgaatttaacttgagacattctcaatccagggctttcctatcagaagatggatctccaacaccggtggaattttttgtttcttcggatgcggcatctgttgttaaacataccaagaaggtgctatttttagaagatgacgatttggctcatatttacgatggtgagttacatattcatagatctagaagagaagtaggcgcatcaatgacaaggtccattcaaactttagagatggagttagctcagatcatgaagggcccttacgaccattttatgcaaaaggaaatctatgagcaaccagaatctactttcaatactatgagaggtagaatcgactatgaaaataataaagtgatattgggtggtttaaaggcatggttaccagttgtcagaagagcacggagactgatcatgatcgcatgcggtacttcttatcattcatgtttggctactcgtgctatcttcgaagaattatcagatatcccagttagtgtggaattagcgtctgactttctggacagaaaatgccctgtcttcagagacgatgtatgcgtgtttgtttcacaaagtggtgaaactgcggataccatgctggctctaaattattgtttagaaagaggagccttaactgtcggaattgttaacagtgttggttcttctatctctcgtgtcacccactgtggtgttcatattaacgctggtcctgaaattggtgttgcctctacaaaagcttatacttcccagtatattgccttagtgatgtttgctctatcgctgtcagatgaccgtgtatcgaaaatagacagaagaattgaaatcattcaaggcttgaagttaatcccgggccaaattaagcaggtattaaagctggaaccaagaataaaaaagctctgtgcgactgaattaaaggatcaaaaatctctattgttattgggtagaggttaccaatttgctgctgctctggaaggtgctttgaagatcaaagaaatttcttatatgcattctgaaggtgttttggcaggtgagttgaagcacggtgtcttggccttggtggacgaaaacttgccaatcattgcttttggtaccagagactctctattccctaaagtagtttcctctattgagcaagttactgcaagaaagggccatccaattattatttgtaacgaaaatgatgaagtgtgggcgcaaaaatctaaatcaatcgacctgcaaaccttagaagttccacaaactgttgattgtttacaaggtctaattaatattattccattacaactaatgtcatattggttggctgttaataaagggattgatgttgattttccaagaaacttggctaaatctgttaccgtcgaataa

SEQ ID NO: 29 - protein, 717 aa, Saccharomyces cerevisiae
MCGIFGYCNYLVERSRGEIIDTLVDGLQRLEYRGYDSTGIAIDGDEADSTFIYKQIGKVSALKEEITKQNPNRDVTFVSHCGIAHTRWATHGRPEQVNCHPQRSDPEDQFVVVHNGIITNFRELKTLLINKGYKFESDTDTECIAKLYLHLYNTNLQNGHDLDFHELTKLVLLELEGSYGLLCKSCHYPNEVIATRKGSPLLIGVKSEKKLKVDFVDVEFPEENAGQPEIPLKSNNKSFGLGPKKAREFEAGSQNANLLPIAANEFNLRHSQSRAFLSEDGSPTPVEFFVSSDAASVVKHTKKVLFLEDDDLAHIYDGELHIHRSRREVGASMTRSIQTLEMELAQIMKGPYDHFMQKEIYEQPESTFNTMRGRIDYENNKVILGGLKAWLPVVRRARRLIMIACGTSYHSCLATRAIFEELSDIPVSVELASDFLDRKCPVFRDDVCVFVSQSGETADTMLALNYCLERGALTVGIVNSVGSSISRVTHCGVHINAGPEIGVASTKAYTSQYIALVMFALSLSDDRVSKIDRRIEIIQGLKLIPGQIKQVLKLEPRIKKLCATELKDQKSLLLLGRGYQFAAALEGALKIKEISYMHSEGVLAGELKHGVLALVDENLPIIAFGTRDSLFPKVVSSIEQVTARKGHPIIICNENDEVWAQKSKSIDLQTLEVPQTVDCLQGLINIIPLQLMSYWLAVNKGIDVDFPRNLAKSVTVE

SEQ ID NO: 30 - DNA, 2142 bp, Candida albicans
  CDS (1)..(2142)
atgtgtggtatttttggttacgtcaatttcttggtcgacaagagtagaggtgaaatcattgataatttaattgaaggtttgcaacgattagaatatagaggttatgattcagcaggcattgctgttgatgggaaattaactaaagatccttctaatggtgatgaagaatatatggattctattattgttaaaactactggtaaagttaaagttttgaaacaaaaaatcattgatgatcaaatcgatagatcggccatttttgataatcatgttggtattgctcatactagatgggctacacatggtcaaccaaaaactgaaaattgtcatcctcataaatcagatccaaagggggaattcattgttgttcataatggtattattactaattatgctgctttaagaaaatatcttttatcaaaaggacatgtttttgaaagtgaaactgatactgaatgtattgctaaattatttaaacatttttatgatttgaatgttaaagctggtgttttccctgatcttaatgaattgactaaacaagttttgcatgaattagaaggttcttatgggttattagttaaatcttatcattatcctggagaagtttgtggtactagaaaaggttctccattattggttggtgttaaaactgataagaaattaaaagttgattttgttgacgttgaatttgaagctcaacagcaacatcgaccacaacaaccacaaatcaatcataatggtgccacttcagctgctgaattgggctttatcccagtggctccaggtgaacaaaatttaagaacttctcaatcaagagctttcctttctgaagatgatttacctatgccagttgaattctttttatcttctgatcctgcatcagtggttcaacacaccaaaaaagttttatttttagaagatgatgatattgctcatatctatgatggggaattacgtattcatagagcttcgactaaatctgctggggaatctactgttagaccaattcaaactttagaaatggaattgaatgaaattatgaaaggcccctataaacattttatgcaaaaagaaattttcgaacaaccagattctgcttttaatactatgagaggtagaattgattttgaaaattgtgttgttacccttggtggattaaaatcatggttatctacaattagaagatgtagaagaatcattatgattgcttgtggtacttcatatcattcatgtttagccacgagatcaatttttgaagaattgacagaaatccccgtttcggttgaattagcttctgatttcttggatagaagatctccagttttcagagatgatacttgtgtatttgtttctcaatcgggtgaaactgccgactccattttggctttacaatattgtttggaaagaggagctttaactgttggtatcgttaactctgttggttcttcaatgtctagacaaacccattgtggggttcatattaatgctgggccagaaattggtgttgcctcaactaaagcttacacatctcaatatattgccttggtgatgtttgccctttctttatctaatgattctatttccagaaagggaagacatgaagaaattattaaaggtttacaaaaaatccctgaacaaattaaacaagttttgaaattagaaaacaagatcaaagatttatgtaatagttcattgaatgatcaaaaatctttattattattaggtagaggttatcaatttgctactgctttagaaggggctttaaaaattaaagaaatttcttatatgcattctgaaggggtattagctggtgaattaaaacatggtatattagcattagtcgatgaagatttaccaattattgcctttgccactagagattcattatttcctaaagttatgtccgctattgaacaagtcactgctagagatggtagaccaattgttatttgtaatgaaggtgatgctattatttctaatgataaagttcatactactttagaagttccagaaaccgttgattgtttacaagggttattaaatgttattccattacaattgattagttattggttggctgtgaatagaggtattgatgttgatttccctcgtaacttggctaaatcagttactgttgagtaa

SEQ ID NO: 31 - protein, 713 aa, Candida albicans
MCGIFGYVNFLVDKSRGEIIDNLIEGLQRLEYRGYDSAGIAVDGKLTKDPSNGDEEYMDSIIVKTTGKVKVLKQKIIDDQIDRSAIFDNHVGIAHTRWATHGQPKTENCHPHKSDPKGEFIVVHNGIITNYAALRKYLLSKGHVFESETDTECIAKLFKHFYDLNVKAGVFPDLNELTKQVLHELEGSYGLLVKSYHYPGEVCGTRKGSPLLVGVKTDKKLKVDFVDVEFEAQQQHRPQQPQINHNGATSAAELGFIPVAPGEQNLRTSQSRAFLSEDDLPMPVEFFLSSDPASVVQHTKKVLFLEDDDIAHIYDGELRIHRASTKSAGESTVRPIQTLEMELNEIMKGPYKHFMQKEIFEQPDSAFNTMRGRIDFENCVVTLGGLKSWLSTIRRCRRIIMIACGTSYHSCLATRSIFEELTEIPVSVELASDFLDRRSPVFRDDTCVFVSQSGETADSILALQYCLERGALTVGIVNSVGSSMSRQTHCGVHINAGPEIGVASTKAYTSQYIALVMFALSLSNDSISRKGRHEEIIKGLQKIPEQIKQVLKLENKIKDLCNSSLNDQKSLLLLGRGYQFATALEGALKIKEISYMHSEGVLAGELKHGILALVDEDLPIIAFATRDSLFPKVMSAIEQVTARDGRPIVICNEGDAIISNDKVHTTLEVPETVDCLQGLLNVIPLQLISYWLAVNRGIDVDFPRNLAKSVTVE

SEQ ID NO: 32 - DNA, 480 bp, Saccharomyces cerevisiae
  CDS (1)..(480)
atgagcttacccgatggattttatataaggcgaatggaagagggggatttggaacaggtcactgagacgctaaaggttttgaccaccgtgggcactattacccccgaatccttcagcaaactcataaaatactggaatgaagccacagtatggaatgataacgaagataaaaaaataatgcaatataaccccatggtgattgtggacaagcgcaccgagacggttgccgctacggggaatatcatcatcgaaagaaagatcattcatgaactggggctatgtggccacatcgaggacattgcagtaaactccaagtatcagggccaaggtttgggcaagctcttgattgatcaattggtaactatcggctttgactacggttgttataagattattttagattgcgatgagaaaaatgtcaaattctatgaaaaatgtgggtttagcaacgcaggcgtggaaatgcaaattagaaaatag

SEQ ID NO: 33 - protein, 159 aa, Saccharomyces cerevisiae
MSLPDGFYIRRMEEGDLEQVTETLKVLTTVGTITPESFSKLIKYWNEATVWNDNEDKKIMQYNPMVIVDKRTETVAATGNIIIERKIIHELGLCGHIEDIAVNSKYQGQGLGKLLIDQLVTIGFDYGCYKIILDCDEKNVKFYEKCGFSNAGVEMQIRK

SEQ ID NO: 34 - DNA, 450 bp, Candida albicans
  CDS (1)..(450)
atgatgttaccacaaggttatacattcagaaaactaaaacttactgattatgataatcaatatttagaaactttaaaagttttgacgacagttggtgaaatttccaaagaagatttcactgaattgtataatcattggtcttcattgccatctatttatcatccatatgtaatcaccaatgcatcaggtatagtggtagccacggggatgttatttgtggagaaaaaattgattcatgaatgtggtaaagttggtcatattgaagatatttcagttgctaaatctgaacaaggtaaaaaattgggatattatttagtcacttcattaaccaaagttgctcaagagaatgattgttacaaagtcattttagattgttctcctgaaaatgttggcttttatgaaaaatgtggttataaagatggtggtgttgaaatggtatgtagattctag

SEQ ID NO: 35 - protein, 149 aa, Candida albicans
MMLPQGYTFRKLKLTDYDNQYLETLKVLTTVGEISKEDFTELYNHWSSLPSIYHPYVITNASGIVVATGMLFVEKKLIHECGKVGHIEDISVAKSEQGKKLGYYLVTSLTKVAQENDCYKVILDCSPENVGFYEKCGYKDGGVEMVCRF

SEQ ID NO: 36 - DNA, 906 bp, Saccharomyces cerevisiae
  CDS (1)..(906)
atgaaaattttcaatacaatacaatctgtgctgttcgcagcattttttctaaaacagggaaattgccttgcgtcaaatgggagtaccgcattgatgggggaagtagatatgcaaacgccctttccagagtggttaacagaatttactaatcttacacaatggcctggaattgacccaccttatattccgctagattacataaatcttactgaagtgccagaattagataggtactatcctggccagtgtcccaaaatttctagagagcagtgctcatttgactgctataactgcatcgatgttgatgatgtaacttcgtgtttcaaactttcccaaacatttgacgacggtccggccccggcgacagaggcattgctcaagaaattgagacaaagaaccactttttttgttctggggataaacactgttaattatcctgatatatatgagcatattttagagaggggtcatttgattggtacacacacgtggtcacatgaattcttgccaagtttatcaaacgaagaaattgtagcccaaattgaatggtcaatttgggctatgaatgccacaggcaaacatttccccaagtattttaggcctccatacggtgcaattgataatagggttagagctatagtaaaacagtttggcctaacggttgtcttgtgggatctcgatacttttgattggaaattaatcactaatgatgatttcagaacagaggaagaaatacttatggacataaatacttggaagggaaaacggaaaggtttgatcttagagcacgatggtgcacgaagaacagttgaggttgctattaaaatcaacgaacttattggtagtgaccaattgacaattgcagaatgtattggtgatacagactacatcgaacgctacgactag

SEQ ID NO: 37 - protein, 301 aa, Saccharomyces cerevisiae
MKIFNTIQSVLFAAFFLKQGNCLASNGSTALMGEVDMQTPFPEWLTEFTNLTQWPGIDPPYIPLDYINLTEVPELDRYYPGQCPKISREQCSFDCYNCIDVDDVTSCFKLSQTFDDGPAPATEALLKKLRQRTTFFVLGINTVNYPDIYEHILERGHLIGTHTWSHEFLPSLSNEEIVAQIEWSIWAMNATGKHFPKYFRPPYGAIDNRVRAIVKQFGLTVVLWDLDTFDWKLITNDDFRTEEEILMDINTWKGKRKGLILEHDGARRTVEVAIKINELIGSDQLTIAECIGDTDYIERYD

SEQ ID NO: 38 - DNA, 939 bp, Saccharomyces cerevisiae
  CDS (1)..(939)
atgagaatacaactaaatacaattgatttgcaatgtattattgcactttcctgtctggggcaatttgttcacgcggaagctaatagggaagatttaaagcagatagactttcaatttcctgtattggaaagggcagctacaaaaacgccttttccggattggcttagtgcatttaccgggttaaaagaatggcctgggttagatccaccttatatacctttagatttcattgatttcagtcaaattccagattataaggaatatgatcaaaaccattgcgacagtgttccaagggactcgtgctctttcgattgccatcactgcaccgaacacgatgatgtgtacacatgttccaaactttcccagacatttgacgatggtccttctgcttccactactaaattattggaccggttgaagcataattccaccttcttcaatttaggtgtcaatatagttcaacatccagatatctatcaaagaatgcaaaaggagggacacttaatcggctcacatacctggtctcacgtatatttgccaaatgtatcgaatgaaaaaattatagctcaaattgaatggtccatctgggcgatgaatgctactggcaaccatacccccaaatggttcagacctccatatggcggaatagataatagagtaagagcaataacaaggcaatttggcttacaagccgtcttatgggatcacgatacttttgattggagcctccttctcaatgattctgtcataactgaacaagaaattcttcaaaatgtaataaactggaacaagtcaggaaccggattaatattagaacacgattcaacggaaaaaactgtcgatcttgccattaaaataaataagttgataggtgatgatcaatcaacagtttctcattgtgtcggcggaattgattacataaaagaattcttgtcctaa

SEQ ID NO: 39 - protein, 312 aa, Saccharomyces cerevisiae
MRIQLNTIDLQCIIALSCLGQFVHAEANREDLKQIDFQFPVLERAATKTPFPDWLSAFTGLKEWPGLDPPYIPLDFIDFSQIPDYKEYDQNHCDSVPRDSCSFDCHHCTEHDDVYTCSKLSQTFDDGPSASTTKLLDRLKHNSTFFNLGVNIVQHPDIYQRMQKEGHLIGSHTWSHVYLPNVSNEKIIAQIEWSIWAMNATGNHTPKWFRPPYGGIDNRVRAITRQFGLQAVLWDHDTFDWSLLLNDSVITEQEILQNVINWNKSGTGLILEHDSTEKTVDLAIKINKLIGDDQSTVSHCVGGIDYIKEFLS

SEQ ID NO: 40 - DNA, 2602 bp, Aspergillus niger
ttcgtcgcctaatgtctcgtccgttcacaaactgaagagcttgaagtggcgagatgtctctgcaggaattcaagctagatgctaagcgatattgcatggcaatatgtgttgatgcatgtgcttcttccttcagcttcccctcgtgcgagtgaggtttggctataaattgaagtggttggtcggggttccgtgaggggctgaagtgcttcctcccttttaggcgcaactgagagcctgagcttcatccccagcatcattacacctcagcaatgtcgttccgatctctactcgccctgagcggcctcgtctgcacagggttggcaaatgtgatttccaagcgcgcgaccttggattcatggttgagcaacgaagcgaccgtggctcgtactgccatcctgaataacatcggggcggacggtgcttgggtgtcgggcgcggactctggcattgtcgttgctagtcccagcacggataacccggactgtatgtttcgagctcagatttagtatgagtgtgtcattgattgattgatgctgactggcgtgtcgtttgttgtagacttctacacctggactcgcgactctggtctcgtcctcaagaccctcgtcgatctcttccgaaatggagataccagtctcctctccaccattgagaactacatctccgcccaggcaattgtccagggtatcagtaacccctctggtgatctgtccagcggcgctggtctcggtgaacccaagttcaatgtcgatgagactgcctacactggttcttggggacggccgcagcgagatggtccggctctgagagcaactgctatgatcggcttcgggcagtggctgcttgtatgttctccacccccttgcgtctgatctgtgacatatgtagctgactggtcaggacaatggctacaccagcaccgcaacggacattgtttggcccctcgttaggaacgacctgtcgtatgtggctcaatactggaaccagacaggatatggtgtgtttgttttattttaaatttccaaagatgcgccagcagagctaacccgcgatcgcagatctctgggaagaagtcaatggctcgtctttctttacgattgctgtgcaacaccgcgcccttgtcgaaggtagtgccttcgcgacggccgtcggctcgtcctgctcctggtgtgattctcaggcacccgaaattctctgctacctgcagtccttctggaccggcagcttcattctggccaacttcgatagcagccgttccggcaaggacgcaaacaccctcctgggaagcatccacacctttgatcctgaggccgcatgcgacgactccaccttccagccctgctccccgcgcgcgctcgccaaccacaaggaggttgtagactctttccgctcaatctataccctcaacgatggtctcagtgacagcgaggctgttgcggtgggtcggtaccctgaggacacgtactacaacggcaacccgtggttcctgtgcaccttggctgccgcagagcagttgtacgatgctctataccagtgggacaagcaggggtcgttggaggtcacagatgtgtcgctggacttcttcaaggcactgtacagcgatgctgctactggcacctactcttcgtccagttcgacttatagtagcattgtagatgccgtgaagactttcgccgatggcttcgtctctattgtggtaagtctacgctagacaagcgctcatgttgacagagggtgcgtactaacagaagtaggaaactcacgccgcaagcaacggctccatgtccgagcaatacgacaagtctgatggcgagcagctttccgctcgcgacctgacctggtcttatgctgctctgctgaccgccaacaaccgtcgtaactccgtcgtgcctgcttcttggggcgagacctctgccagcagcgtgcccggcacctgtgcggccacatctgccattggtacctacagcagtgtgactgtcacctcgtggccgagtatcgtggctactggcggcaccactacgacggctacccccactggatccggcagcgtgacctcgaccagcaagaccaccgcgactgctagcaagaccagcaccagtacgtcatcaacctcctgtaccactcccaccgccgtggctgtgactttcgatctgacagctaccaccacctacggcgagaacatctacctggtcggatcgatctctcagctgggtgactgggaaaccagcgacggcatagctctgagtgctgacaagtacacttccagcgacccgctctggtatgtcactgtgactctgccggctggtgagtcgtttgagtacaagtttatccgcattgagagcgatgactccgtggagtgggagagtgatcccaaccgagaatacaccgttcctcaggcgtgcggaacgtcgaccgcgacggtgactgacacctggcggtgacaatcaatccatttcgctatagttaaaggatggggatgagggcaattggttatatgatcatgtatgtagtgggtgtgcataatagtagtgaaatggaagccaagtcatgtgattgtaatcgaccgacggaattgaggatatccggaaatacagacaccggg

SEQ ID NO: 41 - DNA, 747 bp, Candida albicans
  CDS (1)..(747)
atgagacaagctatattttccaaccctaacgatgctgctgagtatttggcaaactatatcattgccaaaatcaactccacccccagaacatttgttcttggccttccaaccgggtcatcccctgaaggcatttatgccaaattgatcgaagccaacaagcaaggccgtgttagtttcaaaaacgtcgtgaccttcaacatggacgagtatttgggatttgccccatctgacttgcagtcgtaccattatttcatgtacgacaagtttttcaaccatatcgatatcccgcgtgaaaatatccacatcttgaacggattggccgcaaacatcgacgaggagtgtgccaactacgaaaagaaaatcaagcaatacggaagaatcgatttgttcttaggcgggttaggcccagaaggtcatttggcattcaacgaagcgggatcatcaagaaactcaaaaacaagaaaggtcgagttggtcgaaagtaccatcaaggcaaactgcaggtttttcgggaacgacgagagcaaggtccctaaatatgcattgagtgttggtatttccaccatcttggacaactcagacgaaattgccattatcgtgttgggcaaaagtaaacaatttgcattggacaaaactgtaaacgggaaaccaaacgacccaaaatacccatcaagctatttacaagaccacgcaaatgtcttgattgtttgcgataacgctgccgctggattaaagtcaaagttgtag

SEQ ID NO: 42 - protein, 248 aa, Candida albicans
MRQAIFSNPNDAAEYLANYIIAKINSTPRTFVLGLPTGSSPEGIYAKLIEANKQGRVSFKNVVTFNMDEYLGFAPSDLQSYHYFMYDKFFNHIDIPRENIHILNGLAANIDEECANYEKKIKQYGRIDLFLGGLGPEGHLAFNEAGSSRNSKTRKVELVESTIKANCRFFGNDESKVPKYALSVGISTILDNSDEIAIIVLGKSKQFALDKTVNGKPNDPKYPSSYLQDHANVLIVCDNAAAGLKSKL

SEQ ID NO: 43 - DNA, 721 bp, Candida albicans
ttacggtgatgtggctgtggcatggcattgtaccaagtgtgtgatcatcgtagcacctttctcaacagctttcacggctgtgtcataatccgacatcgtatggccaatggaaaacacacagttcttggatttaacaacaggtatcaagtctaaaacaccagcaatttcaggagcagcagtgacaatacacacattatcaaacaagtccccgtaaacttctaacaattttgattcaccctctttggcatcaacaaacgtttcgactgggtggcaccctttcttttgcacattgataaatggcgcctcactgagctccagcgaatctgtctgacttgccaaaacactcctcttgtacattggcaacactttggcatacacttctgggaagcttgatgtgacagtaggacacgtagcagtaaccccagtactcaaatacttggccatagcatctctatagaaccgcttaaactcagcaacatcctctgcggtagactcctcgccaagattggaaaagttcaacccataaataccgttattctgaatatcaataaacccaggtgctaatatttgctgcttcaaatctataacctcactaaccaactcagggtttgctggcggatgacaaattcttttggtggcgttgttgacgtacaagtcagtaaactcatacaactcaccgttgtcgatcaaatgacagtttgtgaatctagtaaatgacatg

SEQ ID NO: 44 - protein, 239 aa, Candida albicans
MSFTRFTNCHLIDNGELYEFTDLYVNNATKRICHPPANPELVSEVIDLKQQILAPGFIDIQNNGIYGLNFSNLGEESTAEDVAEFKRFYRDAMAKYLSTGVTATCPTVTSSFPEVYAKVLPMYKRSVLASQTDSSELSEAPFINVQKKGCHPVETFVDAKEGESKLLEVYGDLFDNVCIVTAAPEIAGVLDLIPVVKSKNCVFSIGHTMSDYDTAVKAVEKGATMITHLVQCHATATSP

SEQ ID NO: 45 - DNA, 3396 bp, Saccharomyces cerevisiae
  CDS (1)..(3396)
atgagtgatcaaaataatcgatcgagaaatgaatatcactcaaaccggaagaatgaaccttcctatgaactccaaaatgcacatagcgggctatttcactcttctaatgaagaattaacaaacaggaaccaaagatataccaatcaaaatgccagcatgggttcattcactccagtccaatctttgcaatttccagaacaatctcagcaaacaaatatgctttataacggtgacgatggcaataataatactatcaatgataacgaacgagacatatatggaggttttgtcaaccaccatcgccagcgtcccccaccagcaactgcagaatacaatgacgtttttaatacgaatagtcaacagctaccgtcggaacatcaatacaataacgtaccttcatatccacttccttcgataaatgtgattcaaaccactccagaactcatacataacggctcacagactatggccacccccatcgaaaggcccttctttaacgaaaacgactactattataataacaggaactctaggacgtcaccgagtattgcttctagtagcgatggttatgcagatcaggaagctaggcccattttggagcaacccaacaataacatgaatagcggtaatattcctcaataccatgaccaaccttttggatacaacaatggttaccatggcctacaggcaaaagattactatgacgatccggagggtggttatattgatcagagaggagatgactatcagattaattcatatttgggtagaaacggtgaaatggttgatccttacgattatgaaaacagtttaagacatatgactcctatggagcgtagagaatatcttcatgatgatagcagacccgtaaacgatggaaaagaagaattagacagtgtgaaaagcggttactctcatagagacttgggggaatatgacaaggatgatttttcaagggatgacgagtacgatgatctcaacactattgataaattacagtttcaagctaatggtgtacctgcatcatcctcggtgtcttctatcggatctaaagaatccgacataatagtaagcaatgataacttaaccgcaaatagagcactaaagagaagcggtactgaaattaggaaattcaaactttggaatggtaattttgttttcgattctccaatcagtaagacgctattggaccaatacgctactacaacagaaaatgcaaacactttaccaaatgagtttaagtttatgagatatcaagcagttacttgcgaacctaatcaacttgcagagaagaatttcacggtgaggcagttgaagtatttaactccaagggaaacggaattgatgctagtagtcacaatgtataatgaagaccatatcctgttaggaagaactttgaaaggtattatggacaatgtcaaatatatggtgaaaaaaaaaaattcaagcacttgggggccggatgcatggaaaaagattgtcgtttgtatcatttcagatggtagatccaaaattaatgaacgctcgctagcattactaagttcgttaggttgttaccaggacgggtttgctaaggatgaaattaatgaaaaaaaagtggcaatgcatgtctacgaacatacgacaatgatcaacatcacaaatatttcggaatcagaggtttcattagaatgcaatcaaggtaccgttccaatacaacttttgttttgtttgaaagagcaaaatcagaaaaaaattaactcacatagatgggcatttgaaggctttgcagaattactgcgtcccaatatcgttacattgttagatgctggtaccatgccaggtaaagattctatttaccagttatggagagagttcaggaatccaaatgttggtggcgcatgtggtgaaataagaactgatttgggtaagagatttgtaaagcttttgaatcctttagttgcatcacagaatttcgaatacaaaatgtccaatattttagacaaaacaaccgagtctaactttggatttattactgttctaccgggggcattctctgcgtataggtttgaagctgtgagaggccaaccattacagaagtacttttatggtgaaattatggaaaatgaaggctttcattttttttcttccaatatgtatcttgctgaagatcgtattttatgctttgaagtggtcacaaaaaaaaattgtaattggattttgaaatactgcagaagttcttatgcttcaacagatgtaccggagagggtccctgaatttattcttcagaggaggcgttggttgaatggttcattttttgctagtgtatattccttttgtcatttttacagagtctggagcagtggtcataatattggtagaaaactccttttgacggttgaatttttttaccttttcttcaatacattgatttcatggttttcattgagttcatttttcctattctttagaattctcactgtttctattgcactggcataccattcagcatttaatgtgttgtccgtcatattcctgtggctttatgggatttgtaccttatcaacattcatactgtcattgggtaataaacctaaaagtactgagaaattttatgttctaacttgcgtcatttttgcggtgatgatgatttacatgatattctgcagtatattcatgagtgtcaaatccttccaaaatatattgaaaaacgataccatcagctttgagggtttgattaccacagaagctttcagggatattgttatctctctgggctccacttattgtttgtacctaatcagttcaattatctatttgcagccatggcatatgttgacaagttttattcagtatattttattgagtccttcttacatcaatgttttgaatatctatgcattttgtaatgtccacgacttatcatggggtacaaagggtgcaatggcaaatccgctgggtaagattaatactacagaagatggtacgttcaaaatggaagttctggtctctagttcagagattcaagcaaactacgataaatatttgaaagttttaaatgacttcgatccaaaatcagaatctcggcctactgagccatcttatgatgaaaaaaagactggctattatgcaaacgttagatctctcgtgattatcttttgggtcatcacaaatttcatcatcgttgctgttgtcttagaaaccggtgggattgcagattatattgctatgaaatccatatcaactgatgacactttagaaactgcaaagaaggcggaaattcccttaatgaccagtaaggcctcaatttattttaatgtaattttatggttagttgcattatcggcattaataaggttcattggttgctcaatatacatgatagtaaggttttttaaaaaggttacatttcgctaa

SEQ ID NO: 46 - protein, 1131 aa, Saccharomyces cerevisiae
MSDQNNRSRNEYHSNRKNEPSYELQNAHSGLFHSSNEELTNRNQRYTNQNASMGSFTPVQSLQFPEQSQQTNMLYNGDDGNNNTINDNERDIYGGFVNHHRQRPPPATAEYNDVFNTNSQQLPSEHQYNNVPSYPLPSINVIQTTPELIHNGSQTMATPIERPFFNENDYYYNNRNSRTSPSIASSSDGYADQEARPILEQPNNNMNSGNIPQYHDQPFGYNNGYHGLQAKDYYDDPEGGYIDQRGDDYQINSYLGRNGEMVDPYDYENSLRHMTPMERREYLHDDSRPVNDGKEELDSVKSGYSHRDLGEYDKDDFSRDDEYDDLNTIDKLQFQANGVPASSSVSSIGSKESDIIVSNDNLTANRALKRSGTEIRKFKLWNGNFVFDSPISKTLLDQYATTTENANTLPNEFKFMRYQAVTCEPNQLAEKNFTVRQLKYLTPRETELMLVVTMYNEDHILLGRTLKGIMDNVKYMVKKKNSSTWGPDAWKKIVVCIISDGRSKINERSLALLSSLGCYQDGFAKDEINEKKVAMHVYEHTTMINITNISESEVSLECNQGTVPIQLLFCLKEQNQKKINSHRWAFEGFAELLRPNIVTLLDAGTMPGKDSIYQLWREFRNPNVGGACGEIRTDLGKRFVKLLNPLVASQNFEYKMSNILDKTTESNFGFITVLPGAFSAYRFEAVRGQPLQKYFYGEIMENEGFHFFSSNMYLAEDRILCFEVVTKKNCNWILKYCRSSYASTDVPERVPEFILQRRRWLNGSFFASVYSFCHFYRVWSSGHNIGRKLLLTVEFFYLFFNTLISWFSLSSFFLFFRILTVSIALAYHSAFNVLSVIFLWLYGICTLSTFILSLGNKPKSTEKFYVLTCVIFAVMMIYMIFCSIFMSVKSFQNILKNDTISFEGLITTEAFRDIVISLGSTYCLYLISSIIYLQPWHMLTSFIQYILLSPSYINVLNIYAFCNVHDLSWGTKGAMANPLGKINTTEDGTFKMEVLVSSSEIQANYDKYLKVLNDFDPKSESRPTEPSYDEKKTGYYANVRSLVIIFWVITNFIIVAVVLETGGIADYIAMKSISTDDTLETAKKAEIPLMTSKASIYFNVILWLVALSALIRFIGCSIYMIVRFFKKVTFR

SEQ ID NO: 47 - protein, 189 aa, Aspergillus niger
  misc_feature (1)..(189): Xaa = any amino acid
DFLFARTMIGVFKNIEYMCSRTSSKTWGKDAWKKIVVCVISDGRAKINPRTRAVLAGLGCYQDGIAKQQVNGKDVTAHIYEYTTQVGLELKGGQVSLKPRTGCPVQMIFCLKEKNQKKINSHRWFFQAFGRVLDPNICVLLDAGTRXGKDSIYHLWKXFDVDPMCGGACGEIKVMXSHGKKLLNPLVAG

SEQ ID NO: 48 - DNA, 2670 bp, Aspergillus fumigatus
  CDS (1)..(2670)
atgggcactccaaggccctattcggcgcattcaccgcaggaaagccgaagcagcttctactcgcagccctcccaatctcctacacagcctacctacggtcgcgatgacgcggaagatcaacaacaatccctccttcgacgcagtttagccagtcccaatggctggtcatacgatgatcccaacgtatctaccgactctctgaggcgatacacattgcatgatccggggataaccgcatttgctcccccgtacccggagtccgaggccgccgatgtacgcagcgcacggatgtcgggatacagtggaatcgagatggatgcctggcaaagacgacaaggggtgaaaccaagtgcgctacggcgatatggaaccaggaagatcaacctcgtccagggttcggtactcagcgttgactacccggtgccgagtgcgattcagaacgcgatccaggcggagtatcgggatgcggaggaagcgtttcatgaagaattcacgcatatgcggtatactgccgccacctgcgacccagatgaattcactctgcgcaacggctacaacctacgtcccgctatgtataaccgtcatacggaacgtctcatcgcgatcacttattataatgaggataaagttctgaccgcccgaactctgcatggagtgatgcaaaacgtccgggacattgtgaacctgaagaagtcagaattttggaacaagggaggaccggcatggcagaagattgtggtgtgtctcgtgtttgatggaattgagccttgcgataagaatactctggatgtcctcgccacgataggtgtgtatcaggatggggtgatgaaaaaagatgtggacgggcgcgagacagtcgcacatattttcgagtatacgacacaattatccgtcaccccgacacagcagctggtcagaccgcagcctaatgatcctagcaatctccccccggtccagatgctattctgcctcaagcagaagaacagcaagaaaatcaattcccaccggtggctgttcaacgcctttagtcgaatcctgaatccggaaatatgcatcctgcttgatgctggcacgaagcccgggagcaaatccttgcttgccctatgggaagcattctataatgacaaaacactgggcggagcatgcggcgaaatccatgccatgctgggcagggggtggcgcaatgtgctgaaccctctagtcgcagcgcagaattttgagtacaaaatttccaatattcttgataaaccgctggaaagcgcctttggctacgtgagtgtgctaccgggtgctttctcggcatatcgctaccgggcgatcatgggccgaccgttggagcaatactttcatggggatcatactttgtccaagcggctgggaaagaagggcattgaggggatgaatatctttaaaaagaacatgtttcttgcagaggatcgcatcctatgctttgaactggtggccaaagctgggtataaatggcatctcacgtatgtgaaagcatccaagggagagacagatgttcccgaggcggcgcccgaatacatcagtcagcggcgacgatggctcaatggctcctttgctgcgagtttgtattccatcatgcacttcggacgcatctataagagtggtcatagcttcgttcgaatgttcttcttgcatattcagatgatttacaactgctgccagctcatcatgacctggttctcgttggcatcctactggctgaccagctcggtcatcatggacctcgtagggacgcccagctcgcataacaagtacaaggcatggccattcggcaacgatgcctcccccattgtcaacttttttgtcaaatatggttatctcttggtgctgatgctccaatttgtgctggctctgggaaaccgccccaaagcctacaccatgtcgttcctctggttttctctggtgcagttctacgtgctgatcctgtccttctacctggtcgctaatgccttcatgggtggcatgatcgactttgatttcgaccaaggcgtgggcaacttcctctcctccttcttcagctcgactggtggagggattgtcctgatcgccttggtgtccacttacggcatttacattgtcgcgagcattctctacatggacccttggcacatcctgaccagttcctgggcatacttcctgggcatgaccacgtcgatcaatattctcatggtgtatgcgttctgcaactggcacgatgtgtcgtggggtacgaaggggtccgataaggcggacgctctgccctcagcgcagacgaagaaggccgacgcgagcaagagcaacttcatcgaggagatcgataaaccgcaggcagacatcgacagccagttcgaggcaacggtcaaacggcggctggccccgtatcaggagcccaaagaagactcgactatcagcctggatgattcctaccgcaactttcggaccagcctggtgttgctctggatcttgagtaatttgctggtctctctgctcattacgagcgacggcatcaggaagatgtgtctgacgaacacatccacaacgcggacgcagtattatttccaagtgattttgtgggcgacagctggactgtctatcttccgattcattgggtcgatctacttcctgggcaagtcgggaatcttatgttgcgtaacgcgtcgatga

SEQ ID NO: 49 - protein, 889 aa, Aspergillus fumigatus
MGTPRPYSAHSPQESRSSFYSQPSQSPTQPTYGRDDAEDQQQSLLRRSLASPNGWSYDDPNVSTDSLRRYTLHDPGITAFAPPYPESEAADVRSARMSGYSGIEMDAWQRRQGVKPSALRRYGTRKINLVQGSVLSVDYPVPSAIQNAIQAEYRDAEEAFHEEFTHMRYTAATCDPDEFTLRNGYNLRPAMYNRHTERLIAITYYNEDKVLTARTLHGVMQNVRDIVNLKKSEFWNKGGPAWQKIVVCLVFDGIEPCDKNTLDVLATIGVYQDGVMKKDVDGRETVAHIFEYTTQLSVTPTQQLVRPQPNDPSNLPPVQMLFCLKQKNSKKINSHRWLFNAFSRILNPEICILLDAGTKPGSKSLLALWEAFYNDKTLGGACGEIHAMLGRGWRNVLNPLVAAQNFEYKISNILDKPLESAFGYVSVLPGAFSAYRYRAIMGRPLEQYFHGDHTLSKRLGKKGIEGMNIFKKNMFLAEDRILCFELVAKAGYKWHLTYVKASKGETDVPEAAPEYISQRRRWLNGSFAASLYSIMHFGRIYKSGHSFVRMFFLHIQMIYNCCQLIMTWFSLASYWLTSSVIMDLVGTPSSHNKYKAWPFGNDASPIVNFFVKYGYLLVLMLQFVLALGNRPKAYTMSFLWFSLVQFYVLILSFYLVANAFMGGMIDFDFDQGVGNFLSSFFSSTGGGIVLIALVSTYGIYIVASILYMDPWHILTSSWAYFLGMTTSINILMVYAFCNWHDVSWGTKGSDKADALPSAQTKKADASKSNFIEEIDKPQADIDSQFEATVKRRLAPYQEPKEDSTISLDDSYRNFRTSLVLLWILSNLLVSLLITSDGIRKMCLTNTSTTRTQYYFQVILWATAGLSIFRFIGSIYFLGKSGILCCVTRR

SEQ ID NO: 50 - DNA, 2751 bp, Aspergillus oryzae
  CDS (1)..(2751)
atggcctaccaaccgcctggtaaagataatggtgctcagtcaccaaactacaacgatagcggtcatcgactggaagacctgccccatggcgccacttatgaagaagaagcttcaacaggactgctttcccaccaacagggcggtcctttcggtggtcctttcgacgaccctcatcagcgtggcacctcgcctgttcgacctacgtcgggatacagcttgactgaaacatacgctccggacgcgggttttcatgacccttacagcacgacgggctcggtttactccggcaactcggcagaaaaccccgcggctgcctttggcgtcccgggtcgtgtagcttctccctacgctcgcagtgaaacatcctccacagaagcatggcgccagcgccaggctccaggaggtggtggcggtggtggcctccgtcgttacgctaccagaaaggtcaagctagttcagggttccgtcctcagtgtcgattaccccgtccccagtgctatccagaatgccatccaagccaagtaccgtaatgacctggagggtggcagcgaggagtttacccacatgcgatacaccgctgcgacatgtgatcccaatgacttcaccctgcacaatggttacaatctgcgtcccgccatgtataacagacacaccgagttgttgattgcgattacctattataacgaagataagacccttaccgctcgtaccttgcacggtgtgatgcagaacattcgcgacattgtgaacctcaagaagtccgagttctggaacaaaggtggacccgcctggcagaaaattgttgtcgctctggtctttgacggtatcgatccttgtgataaggacactttggatgtcctcgccaccatcggtatctatcaggacggtgtcatgaagcgtgacgtcgacggcaaggagaccgtggctcatattttcgaatacacgacccaattgtccgtgacaccgaaccagcaactcatccggcccactgatgatggcccgaccaccttgcccccggtgcagatgatgttctgcttaaagcagaagaacagcaagaagatcaactcacacagatggctgttcaatgccttcggtcgtattttgaatccagaggtttgcatccttctggatgccggtactaagcctggccagaagtccctcctggcgttgtgggagggcttctataacgacaaggatctgggaggtgcttgtggtgaaattcacgcaatgttgggtaaaggctggaagaatctgatcaaccccctcgtcgcggcccagaacttcgagtacaagatcagtaatatcctcgataaacccttggagagttctttcggttatgtcagtgtgttgcctggtgctttctccgcctatcgtttccgtgccatcatgggtcgacccctcgaacaatatttccatggtgatcacactctctcaaaacagctgggtaagaagggtattgagggcatgaacatcttcaagaagaacatgttcttggccgaagatcgtatcctttgtttcgaactggtcgccaaggctggctccaaatggcacttgtcctacatcaaagcctcgaagggtgaaactgacgtgccggaaggtgttgctgaattcatttcccagcgtcgtcgttggttgaacggttcttttgcggccggtctctattcgctcatgcatttcggtcggatgtacaagagtggacataacatcatccgtatgttcttcttgcacattcagatgttgtacaacgttttcaacactatccttacatggttctccctggcatcttactggttgaccaccaccgtcatcatggacttggtcggaacgcccagtgagagcaacggtaacaaaggattccccttcggtaaatcggcgacccctattatcaacacaattgtgaagtatgtctacctcggattgttgctcttgcagttcattctcgctctcggtaaccgccccaagggatcccgcttctcgtacctgacatctttcgtcgtattcggtatcattcaaatctacgttgtcgtcgacgctctgtacttggtggttcgtgcattcacaaacagtgatgcgatagatttcgtcaccgatcaaggtgttggcgagttcctcaagtcgttcttctcgtcttccggcgccggtatcattatcatcgccctggctgctactttcggtctctacttcgtcgcttcgttcatgtacctggacccttggcatatgttcacctcgttccccgcctacatgttcgttcagtcatcttacatcaacgttctcaacgtgtacgcgttcagcaactggcacgatgtctcgtggggtaccaagggttctgataaggccgatgcgctcccttctgcaacgactacgaaggaggatggcggcaaggaagctgtcattgaggaaatcgacaagccccaggctgatattgacagccaatttgaagccactgtcaagcgcgctctcaccccctacgtcccccctgtggagaaggatgagaagtccttggatgattcctacaagagtttccgtacccgtcttgtgacgttgtggatcttcagtaatgccttcttggctgtatgcattaccagtgacggtatggacaagtttggattcacgaacaccgctaccgaccgtacgtcgcgtttcttccaggctctcctgtggtccaacgctgctgtcgcacttgtccgtttcattggtgcctgttggttcctgggtaagacgggtctcatgtgctgcttcgctcgtcggtag

SEQ ID NO: 51 - protein, 916 aa, Aspergillus oryzae
MAYQPPGKDNGAQSPNYNDSGHRLEDLPHGATYEEEASTGLLSHQQGGPFGGPFDDPHQRGTSPVRPTSGYSLTETYAPDAGFHDPYSTTGSVYSGNSAENPAAAFGVPGRVASPYARSETSSTEAWRQRQAPGGGGGGGLRRYATRKVKLVQGSVLSVDYPVPSAIQNAIQAKYRNDLEGGSEEFTHMRYTAATCDPNDFTLHNGYNLRPAMYNRHTELLIAITYYNEDKTLTARTLHGVMQNIRDIVNLKKSEFWNKGGPAWQKIVVALVFDGIDPCDKDTLDVLATIGIYQDGVMKRDVDGKETVAHIFEYTTQLSVTPNQQLIRPTDDGPTTLPPVQMMFCLKQKNSKKINSHRWLFNAFGRILNPEVCILLDAGTKPGQKSLLALWEGFYNDKDLGGACGEIHAMLGKGWKNLINPLVAAQNFEYKISNILDKPLESSFGYVSVLPGAFSAYRFRAIMGRPLEQYFHGDHTLSKQLGKKGIEGMNIFKKNMFLAEDRILCFELVAKAGSKWHLSYIKASKGETDVPEGVAEFISQRRRWLNGSFAAGLYSLMHFGRMYKSGHNIIRMFFLHIQMLYNVFNTILTWFSLASYWLTTTVIMDLVGTPSESNGNKGFPFGKSATPIINTIVKYVYLGLLLLQFILALGNRPKGSRFSYLTSFVVFGIIQIYVVVDALYLVVRAFTNSDAIDFVTDQGVGEFLKSFFSSSGAGIIIIALAATFGLYFVASFMYLDPWHMFTSFPAYMFVQSSYINVLNVYAFSNWHDVSWGTKGSDKADALPSATTTKEDGGKEAVIEEIDKPQADIDSQFEATVKRALTPYVPPVEKDEKSLDDSYKSFRTRLVTLWIFSNAFLAVCITSDGMDKFGFTNTATDRTSRFFQALLWSNAAVALVRFIGACWFLGKTGLMCCFARR

SEQ ID NO: 52 - DNA, 1266 bp, Mucor rouxii
  CDS (1)..(1266)
atgcaaatcaagacattcgccctttcagctgcaattgcacaagttgctactcttgctttagccgacacctccgcaaattactggcaatcatttacttctcaaattaatcccaagaacatctccattccctctattgagcaaacttcatccattgaccccactcaagaatgtgcttactacactcctgatgcttcattgttcacattcaacgcttccgaatggccctctatctgggaagtcgctactaccaatggtatgaatgagtctgccgagttcctcagtgtctacaattctattgactggaccaaggcacccaatatttctgtgcgtacccttgacgctaacggcaacttggataccactggttacaatactgctactgaccctgattgttggtggacagctaccacatgtacctctcccaagatttctgatatcaatgacgatatctccaagtgtcctgaacccgagacttggggtttgacttacgatgatggacctaactgctctcacaacgctttctatgactaccttcaagagcaaaagttgaaggcctccatgttttatattggttccaatgttgttgactggccatacggtgctatgcgtggtgttgttgatggccatcacattgcatcccacacatggtctcaccctcaaatgacaaccaagaccaatcaagaggtccttgctgaattctattatactcaaaaggccatcaagctcgctactggtttgacccctcgttactggcgtcctccttatggtgatatcgatgatcgtgttcgttggattgcctctcaattaggtttaactgctgttatttggaacctcgatactgatgattggtctgctggtgtcactactactgtcgaagctgttgagcaaagttattccgattatattgctatgggtaccaatggtacttttgccaacagtggtaacattgtattgacccatgaaatcaacacaactatgagtctcgctgtcgagaacttgcccaagatcatttctgcctataaacaagtcatcgatgtcgctacctgttacaacatttctcacccttactttgaagactacgaatggaccaatgtcttgaacggcacaaaatcttctgctaccgccagtggatctgctacttctgctagtgcttctggaggcgctactaccgctgccgctcatatccaagcttctactagcggcgccatgtctgtccttcccaacctcgccttgatctctgccttcattgctaccctgttgttttag

SEQ ID NO: 53 - protein, 421 aa, Mucor rouxii
MQIKTFALSAAIAQVATLALADTSANYWQSFTSQINPKNISIPSIEQTSSIDPTQECAYYTPDASLFTFNASEWPSIWEVATTNGMNESAEFLSVYNSIDWTKAPNISVRTLDANGNLDTTGYNTATDPDCWWTATTCTSPKISDINDDISKCPEPETWGLTYDDGPNCSHNAFYDYLQEQKLKASMFYIGSNVVDWPYGAMRGVVDGHHIASHTWSHPQMTTKTNQEVLAEFYYTQKAIKLATGLTPRYWRPPYGDIDDRVRWIASQLGLTAVIWNLDTDDWSAGVTTTVEAVEQSYSDYIAMGTNGTFANSGNIVLTHEINTTMSLAVENLPKIISAYKQVIDVATCYNISHPYFEDYEWTNVLNGTKSSATASGSATSASASGGATTAAAHIQASTSGAMSVLPNLALISAFIATLLF

SEQ ID NO: 54 - DNA, 1293 bp, Gongronella butleri
  CDS (1)..(1293)
atgcgccgccttggacagggcatccaattggtgtccgctgactattggtccaacttcaattcgagcgtgaaccctctcaacgtgaaggtgccccaaatcacacagccacgtcgattgatcacgtcacggagtgcacggtactacacgcctgatccactcgttgatcaccatcacgcccaccggctggggcccaattggctcacggccaccaccaatggcatgaacaccagtgctgaattcaccgccctttacaactcgatcaattgggacaacggcggcccaaacatctcggtgcgcaccttcaacaccgatggctccatgaacactaatggctacgacgtggccaatgatcccgattgttggtggactgtctctggctgcacggtgcccaagctccaggatgtcaatgctgacatttacaagtgtcctgagcccgatacgtggggcttgttctatgacgacggccccaattgctcgcacaatgccttttacaactttttgcaggagcaaaatctgcgcgcttccatgttttacattggttccaacgtcatgaactggccctatggtgccatgcgtggtgtccaagatggccatcacattgctttccacacctggtcccatcagtcattgacgaccctgacgaaccaagaagggctcgccgagttctactacacgcaaaagatgattcacttggccactggtgtgacacctcgctactggcgcgctccctatggtgatgtcgatgatcgtgtgcgctggattgccacgcaattgaacctgacgacgatcctctgggactatgataccaacgactggcaggcaggcgacggtgtgcccgagtccacggtgcaaaacacgtacaatgaattcatccagatgggcaacaatggttcgatggccagcggtggcaacattgtactgacgcacgagatcaacaacacgacgatgcaattggccgtcgagaacatccccaacatgctcaagtcttacaagcacgtcgtcaacgttgccacctgcatgaacatcacgttcccgacatggagcagaccggtgcctttcccttctttagccaatttattgcgcaaaacagcttgggtgcgggcggtgccgccttcaacattaccacgggcgccggggctcagagctcaagctcgttttctcgttgggcgccatgtctttgcccaagccaatgccggtgtcgccgttttgatgacagtccgtgggccgttgcgctttgcatttaaatcttttccaatgggacccccttttctcccttccttatatgttttttga

SEQ ID NO: 55 - protein, 430 aa, Gongronella butleri
MRRLGQGIQLVSADYWSNFNSSVNPLNVKVPQITQPRRLITSRSARYYTPDPLVDHHHAHRLGPNWLTATTNGMNTSAEFTALYNSINWDNGGPNISVRTFNTDGSMNTNGYDVANDPDCWWTVSGCTVPKLQDVNADIYKCPEPDTWGLFYDDGPNCSHNAFYNFLQEQNLRASMFYIGSNVMNWPYGAMRGVQDGHHIAFHTWSHQSLTTLTNQEGLAEFYYTQKMIHLATGVTPRYWRAPYGDVDDRVRWIATQLNLTTILWDYDTNDWQAGDGVPESTVQNTYNEFIQMGNNGSMASGGNIVLTHEINNTTMQLAVENIPNMLKSYKHVVNVATCMNITFPTWSRPVPFPSLANLLRKTAWVRAVPPSTLPRAPGLRAQARFLVGRHVFAQANAGVAVLMTVRGPLRFAFKSFPMGPPFLPSLYVF

SEQ ID NO: 56 - DNA, 1689 bp, Saccharomyces cerevisiae
  CDS (1)..(1689)
atgtcactcctttacatcattcttctattcacacaattcttactactgccaaccgatgcctttgataggtctgctaacacaaatattgctgtttattggggtcaaaactcagcaggaacgcaagaatccttagctacttactgtgaatcttctgatgctgatattttcctattatctttcttgaaccaatttccaacccttggtttgaactttgccaacgcatgctctgatactttttctgatggcttacttcactgcacccagattgctgaagatattgaaacttgccagtccctaggaaagaaagttctattatcattaggtggtgcatctggtagctacctcttttcagatgattctcaagcggaaacttttgcacaaactttatgggatactttcggtgaaggtacaggtgccagtgagagaccatttgactcagcagtcgttgatggttttgattttgatattgaaaacaacaacgaagtaggctatagtgcgttacgtaccaagttaagaactttgtttgccgaaggtacaaagcaatattacctttctgccgcaccacaatgtccatacccggatgcttctgttggtgacttgttggaaaatgcagacattgattttgcgttcatccaattttacaataattactgcagtgtgagtggtcaattcaattgggatacttggttaacctatgctcaaactgtatccccaaataaaaatatcaaactgttcttaggtttacctggttctgcttctgctgctggctctggttatatttctgacacttctttattggaatcaactattgcagatattgcctcttcaagttcttttggtggtattgcgttatgggatgcatctcaagccttttccaacgagctaaatggtgaaccatatgttgagattttgaagaatttgctaacaagtgctagccagaccgccactactacagttgccacctcaaaaacctcagcagcctcaacttcatctgcttcaacttcatctgcttcaacttctcagaaaaagaccacacaatctacgacatctacacaaagtaaaagcaaagttactttatctccaactgcaagcagcgctatcaaaacatcaattactcaaactacaaaaacattgacgagtagcaccaagacaaaatctagtctaggtaccaccacaacagagagcactttaaattcagttgctatcacaagtatgaaaactactctatcttcccaaataaccagtgctgccttggtgacccctcaaacaactactactagcatagtttcttcggccccaattcaaacagctatcactagtactctttcgccagcaacgaagagttcttctgtcgtttccctacagacagctactactagtacgctttccccaacaacgaccagtacaagctcaggtagtacaagctcaggtagtacaagctcagacagtacagctcgtacattggctaaagaattgaatgctcaatatgcggctggtaaattgaacggtaaatctacctgtactgaaggtgaaattgcatgctctgctgatgggaagttcgccgtttgtgatcatagcgcttgggtttacatggaatgtgcttctggaaccacatgttatgcttatgactccggcgactcagtctatacccaatgtaatttctcttatttggaaagcaattacttttaa

SEQ ID NO: 57 - protein, 562 aa, Saccharomyces cerevisiae
MSLLYIILLFTQFLLLPTDAFDRSANTNIAVYWGQNSAGTQESLATYCESSDADIFLLSFLNQFPTLGLNFANACSDTFSDGLLHCTQIAEDIETCQSLGKKVLLSLGGASGSYLFSDDSQAETFAQTLWDTFGEGTGASERPFDSAVVDGFDFDIENNNEVGYSALRTKLRTLFAEGTKQYYLSAAPQCPYPDASVGDLLENADIDFAFIQFYNNYCSVSGQFNWDTWLTYAQTVSPNKNIKLFLGLPGSASAAGSGYISDTSLLESTIADIASSSSFGGIALWDASQAFSNELNGEPYVEILKNLLTSASQTATTTVATSKTSAASTSSASTSSASTSQKKTTQSTTSTQSKSKVTLSPTASSAIKTSITQTTKTLTSSTKTKSSLGTTTTESTLNSVAITSMKTTLSSQITSAALVTPQTTTTSIVSSAPIQTAITSTLSPATKSSSVVSLQTATTSTLSPTTTSTSSGSTSSGSTSSDSTARTLAKELNAQYAAGKLNGKSTCTEGEIACSADGKFAVCDHSAWVYMECASGTTCYAYDSGDSVYTQCNFSYLESNYF

SEQ ID NO: 58 - DNA, 837 bp, Nocardioides sp.
  CDS (1)..(837)
atgcgtctcaagcaccccaccgcccggctcgccctcgccgccctgctggtcgccgtcccgcgcagtgtcgcggccgccggcaccgtccacgccgccccggcccccgccggcgccacccggctcgcggccgtcgggctggacgacccgcacaagaaggacatcgccatgcagctggtgtccagcgccgagaactcctccctcgactggaagtcccagtacaagtacatcgaggacatcaaggacggccgcggctacaccgccggcatcatcggcttctgttccggcaccggcgacatgctcgacctggtcgcagactacaccgacctcaagccgggcaacatcctcgccaagtacctcccggcgctgcgcaaggtcaacggcaccgagtcgcacgccggcctcgcgtccgccttcgagaaggactgggccaccgccgccaaggacagcgtcttccagcaggcccagaacgacgagcgcgaccgctcgtacttcaaccccgcggtcaaccaggccaaggcgagcctgcgcgcgctcggccagttcgcctactacgacgcgatcgtcatgcacggccccggcgacagctcggacagcttcggcggcatccgcaaggccgccatgaagaaggccaagacccccgcccagggccgcgacgaggccacctacctcaaggccttcctggcggcccgcaagaccgtgatgctgaaggaggaggcccactcggacaccagccgggtcgacaccgagcagacggtgttcctcaacgccaagaacttcgacctcaacccgccgctgaagtggaaggtctacggggacagctacgccatcaacagctga

SEQ ID NO: 59 - protein, 278 aa, Nocardioides sp.
MRLKHPTARLALAALLVAVPRSVAAAGTVHAAPAPAGATRLAAVGLDDPHKKDIAMQLVSSAENSSLDWKSQYKYIEDIKDGRGYTAGIIGFCSGTGDMLDLVADYTDLKPGNILAKYLPALRKVNGTESHAGLASAFEKDWATAAKDSVFQQAQNDERDRSYFNPAVNQAKASLRALGQFAYYDAIVMHGPGDSSDSFGGIRKAAMKKAKTPAQGRDEATYLKAFLAARKTVMLKEEAHSDTSRVDTEQTVFLNAKNFDLNPPLKWKVYGDSYAINS